(19) United States
(12) Patent Application Publication
(10) Pub. No.: US 2011/0223176 A1
(43) Pub. Date: Sep. 15, 2011

(54) BASIGIN BINDING PROTEINS

(75) Inventors: Eve H. Barlow, Wellesley, MA (US); Chung-Ming Hsieh, Newton, MA (US); Denise D. Karaoglu Hanzatian, Natick, MA (US); Gillian Ann Kingsbury, Wayland, MA (US); Susan E. Morgan-Lappe, Chicago, IL (US); Edward B. Reilly, Libertyville, IL (US); Fritz G. Buchanan, Antioch, IL (US); Yingchun Li, Buffalo Grove, IL (US)

(73) Assignee: ABBOTT LABORATORIES, Abbott Park, IL (US)

(21) Appl. No.: 13/042,650

(22) Filed: Mar. 8, 2011

Related U.S. Application Data
(60) Provisional application No. 61/312,932, filed on Mar. 11, 2010.
(60) Provisional application No. 61/363,560, filed on Jul. 12, 2010.

(51) Int. Cl. A61K 39/395; C07K 16/28; C07K 16/46; C07H 21/04; C12N 15/63; C12N 5/16; C12N 5/22; A61P 35/00; G01N 33/574

(52) U.S. Cl. 424/143.1; 530/388.1; 530/389.1; 530/388.15; 530/387.3; 536/23.53; 435/320.1; 435/332; 435/7.21

(57) ABSTRACT

Isolated binding proteins, e.g., antibodies, which bind to Basigin (BSG), e.g., human BSG2, and related antibody-based compositions and molecules are disclosed. Also disclosed are pharmaceutical compositions comprising the antibodies, as well as therapeutic and diagnostic methods for using the antibodies.

### RELATED APPLICATIONS

The present application claims priority to prior filed U.S. Provisional Patent Application No. 61/312,932, filed Mar. 11, 2010 and U.S. Provisional Patent Application No. 61/363,560, filed Jul. 12, 2010, the entire contents of each of which are hereby expressly incorporated herein by this reference.

### SEQUENCE LISTING

The instant application contains a Sequence Listing which has been submitted in ASCII format via EFS-Web and is hereby incorporated by reference in its entirety. Said ASCII copy, created on Mar. 4, 2011, is named 10387US.txt and is 125,946 bytes in size.

### FIELD OF THE INVENTION

The present invention relates to the development and use of improved binding proteins, e.g., antibodies, that recognize human Basigin proteins, and specifically to their use in the inhibition, prevention and/or treatment of cancers, tumors, and angiogenesis.

### BACKGROUND OF THE INVENTION

Basigin, also referred to in the art as extracellular matrix metalloproteinase inducer (“EMMPRIN”) and designated cluster of differentiation 147 (CD147), is a cell surface glycoprotein expressed by tumor and many other cell types and is involved in intercellular recognition. Basigin is a type I integral membrane receptor that belongs to the immunoglobulin superfamily and has numerous ligands, including the cyclophilin (CyP) proteins Cyp-A and CyP-B and certain integrins (Berditchevski, et al. (1997) J. Biol. Chem., 272:46, 29174-29180; Yurchenko, et al. (2001) Biochem. Biophys. Res. Commun, 288:4, 786-788; Yurchenko, et al. (2002) J. Biol. Chem., 277:25, 22959-22965).

The basigin protein exists in several isoforms. The human basigin protein (“hBSG2” or “BSG2”) contains 269 amino acids and is characterized by the presence of two extracellular immunoglobulin-like domains, a single transmembrane domain possessing a charged amino acid and a short cytoplasmic tail containing a basolateral membrane targeting motif (Deora, et al. (2004) Mol. Biol. Cell, 15:9, 4148-4165; Miyauchi, et al. (1991) J. Biochem., 110:5, 770-774). It is expressed as several differentially spliced isoforms encoded by a single gene found on chromosome 19p13.3 (Guo, et al. (1998) Gene, 220:1-2, 99-108; Hanna, et al. (2003) BMC Biochem., 4:17; Kaname, et al. (1993) Cytogenet. Cell. Genet., 63:3-4, 195-197); (Accession Nos. NM_{—}198591.1 (isoform 4), NM_{—}001728.2 (isoform 1), and NM_{—}198589.2 (isoform 2)).

BSG has a variety of functions, including inducing matrix metalloproteinase production and regulating spermatogenesis, monocarboxylate transporter expression, the responsiveness of lymphocytes, embryo implantation, neural network formation, and tumor progression. In particular, BSG is involved with the expression of molecules involved in tissue remodeling and angiogenesis, and as such is a target for the development of therapeutic strategies to inhibit tumor metastasis.

There is a need in the art for improved antibodies capable of binding BSG, e.g., BSG2. The present invention provides a novel family of binding proteins, e.g., antibodies, and fragments thereof, capable binding BSG2 with high affinity.

### SUMMARY OF THE INVENTION

This invention pertains to BSG2 binding proteins, particularly anti-BSG2 antibodies, or antigen-binding portions thereof. In particular, the present invention provides a novel class of murine and humanized monoclonal antibodies which bind to BSG2 and inhibit various BSG2 functions. For example, the antibodies described herein are capable of binding to BSG2 and inhibiting angiogenesis. Monoclonal antibodies of the present invention, thus, are useful for treating and diagnosing a variety of diseases, such as cancers associated with BSG2 mediated angiogenesis.

In one aspect, the invention is directed to an isolated monoclonal antibody or antigen binding portion thereof that binds to BSG2 and inhibits a BSG2 mediated activity. In another aspect, the invention is directed to an isolated monoclonal antibody or antigen binding portion thereof that binds to BSG2, wherein the antibody or antigen binding portion thereof exhibits one or more of the following properties: (i) inhibition of spermatogenesis; (ii) inhibition of expression of monocarboxylate transporter expression; (iii) inhibition of lymphocyte responsiveness; (iv) inhibition of embryo implantation; (v) inhibition of formation of neural network; (vi) inhibition of tumor progression; (vii) inhibition of tumor angiogenesis; and (viii) inhibition of production matrix metalloproteinase. In another aspect, the invention is directed to an isolated monoclonal antibody or antigen binding portion thereof, comprising a heavy chain (HC) immunoglobulin variable domain sequence and a light chain (LC) immunoglobulin variable domain sequence, wherein the antibody or antigen binding portion thereof binds to BSG2 and (A) the HC immunoglobulin variable domain sequence comprises one or more of the following properties: i) a HC CDR1 that comprises the amino acid sequence: NFWMD (SEQ ID NO:48); ii) a HC CDR2 that comprises an amino acid sequence as follows: (G/E)-I-R-L-K-S-(Y/T)-N-Y-A-T-H-Y-A-E-S V-K-G (SEQ ID NO: 95); or iii) a HC CDR3 that comprises an amino acid sequence as follows: (W/T)-(D/S)-(G/T)-(A/G)-Y (SEQ ID NO:96); and B) the LC immunoglobulin variable domain sequence comprises one or more of the following properties: i) a LC CDR1 that comprises an amino acid sequence as follows: K-A-S-Q-(D/S)-V-S-(T/N)-D-V-A (SEQ ID NO:97); ii) a LC CDR2 that comprises an amino acid sequence as follows: (S/Y)-A-S-(Y/N)—R—Y-T (SEQ ID NO: 98); or iii) a LC CDR3 that comprises an amino acid sequence as follows: Q-Q-(H/D)-Y—S-(T/S)-P-(F/Y)-T (SEQ ID NO:99).

In particular embodiments, the antibody or antigen binding portion thereof binds to BSG2 with a K_{D} of at least about 8 nM or better, as measured by a surface plasmon resonance assay or a cell binding assay.

In various embodiments of each of the foregoing aspects of the invention, the antibody or antigen binding portion thereof dissociates from human BSG2 extracellular domain with a k_{off} rate constant of 1×10⁻¹ s⁻¹or less, 1×10⁻² s⁻¹or less, 1×10⁻⁴ s⁻¹ or less, 1×10⁻⁵ s⁻¹ or less, or 1×10⁻⁶ s⁻¹ or less, as determined by surface plasmon resonance.

In a further embodiments of each of the foregoing aspects of the invention, the antibody or antigen-binding portion thereof binds to human BSG2 extracellular domain with a K_{D} of 1×10⁻⁵M or less, of 1×10⁻⁶M or less, of 1×10⁻⁷M or less, of 1×10⁻⁸M or less, or of 1×10⁻⁹M or less, as determined by surface plasmon resonance.

In other embodiments of the foregoing aspects of the invention, the antibody or antigen-binding portion thereof binds to human BSG2 with an EC₅₀ of less than 2 nM, 1.9 nM, 1.8 nM, 1.7 nM, 1.6 nM, 1.5 nM, 1.4 nM, 1.3 nM, 1.2 nM, 1.1 nM, 1.0 nM, 0.9 nM, 0.8 nM, 0.7 nM, 0.6 nM, or 0.5 nM, as measured by electrochemeluminescence (ECL).

In further embodiments of the foregoing aspects of the invention, the antibody or antigen-binding portion thereof binds to human BSG2 with a K_{D} of 5 nM, 4.5 nM, 4 nM, 3.5 nM, 3 nM, 2.5 nM, 2 nM, 1.5 nM, 1 nM or 0.5 nM or less, as determined by a receptor binding assay.

In additional embodiments, the antibody or antigen-binding portion thereof induces CDC or ADCC mediated killing of tumor cells, for example, by at least 10%, 15%, 20%, 25%, 30%, 35%, 40%, 45%, 50%, 55%, 60%, 65%, 70%, 75%, 80%, 85%, 90%, 95% or 100% killing of tumor cells, such as pancreatic or hepatocellular cancer cells, as measured by a complement-dependent cytotoxicity assay.

In further embodiments, the antibody or antigen-binding portion thereof results in at least 10%, 15%, 20%, 25%, 30%, 35%, 40%, 45%, 50%, 55%, 60%, 65%, 70%, 75%, 80%, 85%, 90%, 95% or 100% killing of hepatocellular cancer cells, as measured by a complement-dependent cytotoxicity assay upon exposure of hepatocellular cancer cells to 10 μg/ml of the antibody or antigen binding portion thereof.

In additional embodiments, the antibody or antigen-binding portion thereof decreases Akt phosphorylation and/or disrupts mitochondrial membrane potential in human cancer cells.

In further embodiments, the antibody or antigen-binding portion thereof inhibits tumor growth at least 10%, 15%, 20%, 25%, 30%, 35%, 40%, 45%, 50%, 55%, 60%, 65%, 70%, 75%, 80%, 85%, 90%, 95% or 100% tumor growth inhibition as measured by a human hepatocellular, human pancreatic cancer or human lymphoma xenograft model.

In certain embodiments, the antibody or antigen binding portion thereof binds to human BSG2. In additional embodiments, the antibody, or antigen binding portion thereof is capable of modulating a biological function of one or more targets selected from the group consisting of a cyclophilin, integrin, VEGF, MMP, Aid, and ErbB2.

In another aspect, the invention is directed to an isolated monoclonal antibody or antigen binding portion thereof that binds to BSG2, wherein the antibody or antigen binding portion thereof includes (a) a heavy chain variable region comprising an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to the, e.g., to the entire, heavy chain variable region amino acid sequence set forth in SEQ ID NO: 20, 26-28, 38-40, 59 and 75; (b) a light chain variable region comprising an amino acid sequence at least 80%, 81%, 82%, 83%, 84%, 85%, 86%, 87%, 88%, 89%, 90%, 91%, 92%, 93%, 94%, 95%, 96%, 97%, 98% or 99% identical to the, e.g., to the entire, light chain variable region amino acid sequence set forth in SEQ ID NO:22, 32-35, 42-43, 45-46, 63 and 79. For example, the invention is directed to an isolated monoclonal antibody or antigen binding portion thereof that binds to BSG2, wherein the antibody or antigen binding portion thereof includes a heavy chain variable region comprising an amino acid sequence at least 95% identical to the heavy chain variable region amino acid sequence set forth in SEQ ID NO:20, 26-28, 38-40, 59 and 75; and/or a light chain variable region comprising an amino acid sequence at least 95% identical to the, e.g., to the entire, light chain variable region amino acid sequence set forth in SEQ ID NO:22, 32-35, 42-43, 45-46, 63 and 79. In yet another aspect, the invention is directed to an isolated antibody or antigen binding portion thereof that binds to the epitope which is same or overlapping with the epitope bound by the any of the foregoing described antibodies.

In another aspect, the invention is directed to an isolated monoclonal antibody or antigen binding portion thereof that binds to BSG2, wherein the antibody or antigen binding portion thereof includes a heavy chain variable region comprising CDR1, CDR2, and CDR3 sequences; and a light chain variable region comprising CDR1, CDR2, and CDR3 sequences, wherein the heavy chain variable region CDR3 sequence includes an amino acid sequence selected from the group consisting of SEQ ID NO:52, 62, 78 and conservative amino acid substitutions thereof. In various embodiments, the antibody or antigen binding portion thereof may further include (a) a light chain variable region CDR3 sequence including an amino acid sequence selected from the group consisting of SEQ ID NO:58, 66, 82 and conservative sequence modifications thereof; (b) a heavy chain variable region CDR2 sequence including an amino acid sequence selected from the group consisting of SEQ ID NOs: 50, 61, 77 and conservative sequence modifications thereof; (c) a light chain variable region CDR2 sequence including an amino acid sequence selected from the group consisting of SEQ ID NOs: 56, 65, 81 and conservative sequence modifications thereof; (d) a heavy chain variable region CDR1 sequence including an amino acid sequence selected from the group consisting of SEQ ID NOs:48, 60, 76 and conservative sequence modifications thereof; and/or (e) a light chain variable region CDR1 sequence including an amino acid sequence selected from the group consisting of SEQ ID NOs: 54, 64, 80 and conservative sequence modifications thereof.

In another aspect, the invention is directed to an isolated monoclonal antibody or antigen binding portion thereof that binds to BSG2 and includes a heavy chain variable region CDR1 comprising SEQ ID NO:48; a heavy chain variable region CDR2 comprising SEQ ID NO:50; a heavy chain variable region CDR3 comprising SEQ ID NO: 52; a light chain variable region CDR1 comprising SEQ ID NO: 54; a light chain variable region CDR2 comprising SEQ ID NO: 56; and a light chain variable region CDR3 comprising SEQ ID NO: 58. In yet another aspect, the invention is directed to an isolated monoclonal antibody or antigen binding portion thereof that binds to BSG2 and includes a heavy chain variable region CDR1 comprising SEQ ID NO:60; a heavy chain variable region CDR2 comprising SEQ ID NO:61; a heavy chain variable region CDR3 comprising SEQ ID NO: 62; a light chain variable region CDR1 comprising SEQ ID NO: 64; a light chain variable region CDR2 comprising SEQ ID NO: 65; and a light chain variable region CDR3 comprising SEQ ID NO: 66. In yet another aspect, the invention is directed to an isolated monoclonal antibody or antigen binding portion thereof that binds to BSG2 and includes a heavy chain variable region CDR1 comprising SEQ ID NO:76; a heavy chain variable region CDR2 comprising SEQ ID NO:77; a heavy chain variable region CDR3 comprising SEQ ID NO:78; a light chain variable region CDR1 comprising SEQ ID NO: 80; a light chain variable region CDR2 comprising SEQ ID NO:81; and a light chain variable region CDR3 comprising SEQ ID NO:82.

In a further aspect, the present invention is directed to an isolated monoclonal antibody or antigen binding portion thereof that binds to BSG2 and includes a heavy chain variable region including CDR1, CDR2, and CDR3 sequences; and a light chain variable region including CDR1, CDR2, and CDR3 sequences, wherein the heavy chain variable region CDR3 sequence includes an amino acid sequence which is at least 90%, 91%, 92%, 93%, 94%, 95%, 96%, 97%, 98% or 99% identical to the, e.g., to the entire, amino acid sequence selected from the group consisting of SEQ ID NOs: 52, 62 and 78. In particular embodiments of the foregoing aspect, the antibody further includes (a) a light chain variable region CDR3 sequence comprising an amino acid sequence which is at least 90%, 91%, 92%, 93%, 94%, 95%, 96%, 97%, 98% or 99% identical to the, e.g., to the entire, amino acid sequence selected from the group consisting of SEQ ID NOs:58, 66 and 82; (b) a heavy chain variable region CDR2 sequence comprising an amino acid sequence which is at least 90%, 91%, 92%, 93%, 94%, 95%, 96%, 97%, 98% or 99% identical to the, e.g., to the entrie, amino acid sequence selected from the group consisting of SEQ ID NOs:50, 61 and 77; (c) a light chain variable region CDR2 sequence comprising an amino acid sequence which is at least 90%, 91%, 92%, 93%, 94%, 95%, 96%, 97%, 98% or 99% identical to the, e.g., to the entire, amino acid sequence selected from the group consisting of SEQ ID NOs: 56, 65 and 81; (d) a heavy chain variable region CDR1 sequence comprising an amino acid sequence which is at least 90%, 91%, 92%, 93%, 94%, 95%, 96%, 97%, 98% or 99% identical to the, e.g., to the entire, amino acid sequence selected from the group consisting of SEQ ID NOs:48, 60 and 76; and/or (e) a light chain variable region CDR1 sequence comprising an amino acid sequence which is at least 90%, 91%, 92%, 93%, 94%, 95%, 96%, 97%, 98% or 99% identical to the, e.g., to the entire, amino acid sequence selected from the group consisting of SEQ ID NOs: 54, 64 and 80.

In particular embodiments, the antibody or antigen binding portion thereof of the present invention includes a light chain variable region from human VH3 germline gene. For example, the heavy chain variable region comprises a VH3-73 human germline acceptor sequence. In addition, the heavy chain may include hJH4 or hJH6 as the acceptor human FR4 sequence. In a particular embodiment, the antibody, or antigen binding portion includes a VH3-73 human germline acceptor sequence and at least one framework change selected from the group consisting of V48I, G49A, N76S, A78V, R94A, R94D, K19R, S41P, K83R, T84A and combinations thereof.

Alternatively or in addition, the antibody or antigen binding portion thereof of present invention includes a light chain variable region from human Vk1 or Vk3 germline gene, for example an O8/O18 or 3-15/L2 acceptor sequence. In a further embodiment, the light chain further includes hJk2 or hJk4 as the acceptor human FR4 sequence. In a particular embodiment, the light chain variable region comprises an O8/O18 human germline acceptor sequence and at least one framework change selected from the group consisting of A43S, Y87F, Q3V, I83F, and combinations thereof. In an alternative embodiment, the light chain variable region comprises a 3-15/L2 human germline acceptor sequence and at least one framework change selected from the group consisting of A43S, I58V, Y87F and combinations thereof.

In various embodiments, the antibody, or antigen binding portion thereof, is selected from the group consisting of a Fab, Fab′2, ScFv, SMIP, affibody, avimer, nanobody, and domain antibody. In certain embodiments, the antibody isotype is selected from the group consisting of an IgG1, an IgG2, an IgG3, an IgG4, an IgM, an IgA1, an IgA2, an IgAsec, an IgD, and an IgE antibody.

In various embodiments, the antibody is selected from the group consisting of a human antibody, a humanized antibody, a bispecific antibody and a chimeric antibody. In particular embodiments, the antibody is a humanized antibody.

In a further aspect, the present invention is directed to an isolated monoclonal antibody or antigen binding portion thereof that binds to BSG2 and comprises a variable heavy chain sequence selected from the group consisting of SEQ ID NOs:27 and 28, and a variable light chain sequence selected from the group consisting of SEQ ID NOs:33, 34 and 35. In a particular embodiment, the antibody or antigen binding portion thereof includes a variable heavy chain sequence comprising SEQ ID NO:28 and a variable light chain sequence comprising SEQ ID NO:35. In certain embodiments, the antibody or antigen binding portion thereof is of the IgG1 isotype.

In a further aspect, the present invention is directed to an isolated monoclonal antibody or antigen binding portion thereof that binds to BSG2 and includes a variable heavy chain sequence selected from the group consisting of SEQ ID NOs:38, 39 and 40, and a variable light chain sequence selected from the group consisting of SEQ ID NOs:42, 43, 45 and 46.

In a particular aspect, the invention is directed to a composition including the antibody, or antigen binding portion thereof, of the invention and a pharmaceutically acceptable carrier. In another aspect, the invention is directed to a composition including two or more antibodies, or an antibody binding portion thereof, wherein the antibodies, or antigen binding portion thereof, bind to different epitopes on BSG2.

In another aspect, the invention is directed to an isolated nucleic acid molecule encoding a heavy chain variable region of an antibody that binds BSG2, wherein said antibody includes a heavy chain variable region sequence at least 90%, 91%, 92%, 93%, 94%, 95%, 96%, 97%, 98% or 99% identical to a, e.g., to an entire, sequence selected from the group consisting of SEQ ID NOs:20, 26-28, 38-40, 59 and 75. In another aspect, the invention is directed to an isolated nucleic acid molecule encoding a light chain variable region of an antibody that binds BSG2, wherein said antibody includes a light chain variable region sequence at least 90%, 91%, 92%, 93%, 94%, 95%, 96%, 97%, 98% or 99% identical to a, e.g., to an entire, sequence selected from the group consisting of SEQ ID NOs:22, 32-35, 42-43, 45-46, 63 and 79. In yet another aspect, the invention is directed to an isolated nucleic acid molecule encoding a heavy chain variable region of an antibody that binds BSG2, including a nucleotide sequence that hybridizes under highly stringent conditions to a nucleotide sequence encoding a heavy chain variable region selected from the group consisting of SEQ ID NOs:20, 26-28, 38-40, 59 and 75. In yet a further aspect, the invention is directed to an isolated nucleic acid molecule encoding a light chain variable region of an antibody that binds BSG2, including a nucleotide sequence that hybridizes under highly stringent conditions to a nucleotide sequence encoding a light chain variable region selected from the group consisting of SEQ ID NOs:22, 32-35, 42-43, 45-46, 63 and 79.

In various aspects, the invention is directed to an expression vector including one of the above-described nucleic acid molecules or, alternatively, a host cell including one of the above-described nucleic acid molecules. In another aspect, the invention provides a transgenic non-human mammal or a transgenic plant which expresses a monoclonal antibody or antigen binding portion thereof that binds the same epitope as the antibody or antigen binding portion as described herein.

In another aspect, the present invention provides a hybridoma which produces an antibody or antigen binding portion as described herein.

In yet another aspect, the present invention is directed to a kit including one or more isolated monoclonal antibodies, or antigen binding portions thereof, as described herein and, optionally, instructions for use in treating or diagnosing a disease associated with BSG2 activity, for example, a disease associated with abnormal angiogenesis such as cancer, neovascular disease, ocular disease, atherosclerosis, hemangiomas, chronic inflammation or arthritis.

In yet another aspect, the present invention is directed to a method of inhibiting abnormal angiogenesis in a subject, by administering to the subject an isolated monoclonal antibody, or antigen binding portion thereof, as described herein, in an amount sufficient to inhibit BSG2 activity. In a further aspect, the present invention is directed to a method of treating a BSG2 mediated disease, for example, cancer, in a subject, by administering to the subject a therapeutically effective amount of an isolated monoclonal antibody, or antigen binding portion thereof, of the invention. For example, the cancer may be pancreatic cancer, liver cancer, lymphoma, melanoma, breast cancer, ovarian cancer, renal carcinoma, gastrointestinal/colon cancer, lung cancer, clear cell sarcoma or prostate cancer. In certain embodiments, the subject is human.

In various embodiments, the antibody, or antigen binding portion thereof, is administered intravenously, intramuscularly, or subcutaneously to the subject. In certain embodiments, the antibody, or antigen binding portion thereof, is administered in combination with a second therapeutic agent, for example, a second antibody or antigen binding portion thereof. The second therapeutic agent may be an anti-cancer agent, such as an antibody, a small molecule, an antimetabolite, an alkylating agent, a topoisomerase inhibitor, a microtubule-targeting agent, a kinase inhibitor, a protein synthesis inhibitor, an immunotherapeutic, a hormone or analog thereof, a somatostatin analog, a glucocorticoid, an aromatose inhibitor, and an mTOR inhibitor.

In a further aspect, the present invention is directed to a method of diagnosing a cancer associated with BSG2 in a subject, comprising (a) contacting ex vivo or in vivo cells from the subject with an isolated monoclonal antibody, or antigen binding portion thereof that binds to BSG2, and (b) measuring the level of binding to BSG2 on the cells, wherein abnormally high levels of binding to BSG2 indicate that the subject has a cancer associated with BSG2.

### BRIEF DESCRIPTION OF THE DRAWINGS

FIG. 1 shows the humanized VH design version of murine antibody 3A3.

FIG. 2 shows the humanized VL design version of murine antibody 3A3.

FIG. 3 depicts the variable region sequence annotations with Kabat numbering for the 2C1 heavy chain, as described in Example 7.1.

FIG. 4 depicts an alignment between each possible acceptor human FR4 sequence as compared to the 2C1 FR4 sequence, as described in Example 7.1.

FIG. 5 depicts the 2C1VH sequence against suggested human VH framework sequence acceptors, as described in Example 7.1.

FIG. 6 depicts the alignment of profile VH sequences with human VH sequences in the Align X program of Vector NTI suite, as described in Example 7.1.

FIG. 7 depicts an alignment of the two IGHV3-73 sequences, IGHV3-73*01 and *02, demonstrating that the sequences are identical.

FIG. 8 depicts an alignment of VH3-73JH6.5 (SEQ ID NO:37) with the humanized 2C1VH sequences using VH3-73 as the acceptor sequence, i.e., h2C1VH.1 (SEQ ID NO:38), h2C1VH.1a (SEQ ID NO:39) and h2C1VH.1b (SEQ ID NO:40), as described in Example 7.1.

FIG. 9 depicts the predicted immunogenicity of the humanized 2C1VH sequences using the EpiVax database, as described in Example 7.1.

FIG. 10 depicts a cluster analysis of the humanized 2C1VH sequences. “Cluster Sequences” are disclosed as SEQ ID NOS 110, 110, 110-113, 113, 113-116, 115, 117, 166-167, 167, and 167 respectively, in order of appearance.

FIG. 11 depicts an alignment of the murine 2C1 VH against the humanized 2C1VH sequences using VH3-73 as the acceptor sequence, i.e., h2C1VH.1 (SEQ ID NO:38), h2C1VH.1a (SEQ ID NO:39) and h2C1VH.1b (SEQ ID NO:40), as described in Example 7.1.

FIG. 12A depicts the identity and similarity between the 2C1 VH with the humanized 2C1 VH sequences. FIG. 12B depicts the identity and similarity between VH3-73JH6.5, 2C1 VH and the humanized 2C1 VH sequences.

FIG. 13 depicts the variable region sequence annotations with Kabat numbering for the 2C1 variable light chain as described in Example 7.2.

FIG. 14 depicts an alignment of possible human light chain FR4 sequences as compared to the 2C1 variable light chain FR4 sequence, as described in Example 7.2.

FIG. 15 depicts an alignment of the 2C1 variable light chain against suggested human VL framework acceptors, as described in Example 7.2.

FIG. 16 depicts the identities and similarities of the profile sequences aligned with human variable light chain sequences in the Align X program of the Vector NTI suite, as described in Example 7.2.

FIG. 17 depicts an alignment of human Vk1 germline sequences to identify potential framework residues in O8/O18 that should be changed to Vk1 consensus to minimize the immunogenicity potential of the humanized sequence, as described in Example 7.2.

FIG. 18 depicts an alignment of human Vk3 germline sequences to identify potential framework residues in IGKV3-15/L2 that should be changed to Vk3 consensus to minimize the immunogenicity potential of the humanized sequence, as described in Example 7.2.

FIG. 19 depicts the alignment of O18Jk4 (SEQ ID NO:41) with each of the humanized 2C1 VL sequences using O8/O18 as the acceptor sequence, i.e., h2C1VL.1 (SEQ ID NO:42) and h2C1VL.1a (SEQ ID NO:43), as described in Example 7.2.

FIG. 20 depicts the predicted immunogenicity of the humanized 2C1 VL sequences (with the O8/O18 acceptor sequence) using the EpiVax database, as described in Example 7.2. Based on the results depicted therein, the humanized 2C1 VL sequences with O8/O18 acceptor sequences do not appear to be immunogenic.

FIG. 21 depicts the cluster selection analysis of the humanized 2C1 VL sequences (with the O8/O18 acceptor sequence), as described in Example 7.2. Based on the results depicted therein, the FR2 to CDR2 to FR3 region is a potential T cell epitope.

FIG. 22 depicts an alignment of L2Jk4 (SEQ ID NO:44) with the 2C1 VL sequences designed using 3-15/L2 as the acceptor sequence, i.e., h2C1VL.2 (SEQ ID NO:45) and h2C1VL.2a (SEQ ID NO:46), as described in Example 7.2.

FIG. 23 depicts the predicted immunogenicity of the humanized 2C1 VL sequences (with the 3-15/L2 acceptor sequence) using the EpiVax database, as described in Example 7.2. Based on the results depicted therein, the grafted H2C1 VL sequences with 3-15/L2 acceptor sequences do not appear to be immunogenic.

FIG. 24 depicts the cluster selection analysis of the grafted 2C1 VL sequences (with the 3-15/L2 acceptor sequence), as described in Example 7.2. Based on the results depicted therein, the FR2 to CDR2 to FR3 region is a potential T cell epitope.

FIG. 25 depicts an alignment of each of the 2C1VL against the humanized 2C1 VL sequences using either O8/O18 or 3-15/L2 as the acceptor, i.e., h2C1VL.1 (SEQ ID NO:42), h2C1VL.1a (SEQ ID NO:43), h2C1VL.2 (SEQ ID NO:45) and h2C1VL.2a (SEQ ID NO:46), as described in Example 7.2.

FIG. 26A depicts the identities and similarities of each of these generated sequences as compared to the 2C1VL sequence. Identities and similarities of the humanized 2C1VL sequences using O8/O18 acceptor sequences as compared to O18Jk4 are set forth in FIG. 26B. Identities and similarities of the humanized 2C1VL sequences using 3-15/L2 acceptor sequences as compared to L2Jk4 are set forth in FIG. 26C.

### DETAILED DESCRIPTION OF THE INVENTION

This invention pertains to Basigin (BSG2) binding proteins, particularly anti-BSG2 antibodies, or antigen-binding portions thereof. Various aspects of the invention relate to antibodies and antibody fragments, and pharmaceutical compositions thereof, as well as nucleic acids, recombinant expression vectors and host cells for making such antibodies and fragments. Methods of using the antibodies of the invention to detect BSG2; to inhibit or enhance BSG2 signal transduction, either in vitro or in vivo; and to regulate BSG2-related functions are also encompassed by the invention.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including,” as well as other forms of the term, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, pathology, oncology, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the present invention may be more readily understood, select terms are defined below.

The term “polypeptide,” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide,” as used herein, refers to a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering,” as used herein, refers to the process of rendering a chemical species such as a polypeptide substantially free of naturally associated components by isolation, e.g., using protein purification techniques well known in the art.

The term “BSG2” or “basigin” or “BSG” refers a plasma membrane protein that is widely expressed and implicated in a variety of physiological and pathological activities. As used herein, the term “human basigin-2” (abbreviated herein as “hBSG2”) is understood to refer to the prototypical 269 amino acid basigin isoform having the amino acid sequence of SEQ ID NO:1 (NCBI Accession No. NP940991) shown in Table 1, and its related isoform (SEQ ID NO:2). Other isoforms of BSG include the amino acid sequence of SEQ ID NO:36 (isoform 4), also shown in Table 1.

TABLE 1
BSG Sequences.

| | Sequence | |
|---|---|---|
| Protein | Identifier | Sequence |
| hBSG2 | SEQ ID NO: 1 | MAAALFVLLGFALLGTHGASGAAGTVFTTV |
| (isoform 2, short) | | EDLGSKILLTCSLNDSATEVTGHRWLKGGV |
| | | VLKEDALPGQKTEFKVDSDDQWGEYSCVFL |
| | | PEPMGTANIQLHGPPRVKAVKSSEHINEGE |
| | | TAMLVCKSESVPPVTDWAWYKITDSEDKAL |
| | | MNGSESRFFVSSSQGRSELHIENLNMEADP |
| | | GQYRCNGTSSKGSDQAIITLRVRSHLAALW |
| | | PFLGIVAEVLVLVTIIFIYEKRRKPEDVLD |
| | | DDDAGSAPLKSSGQHQNDKGKNVRQRNSS |
| hBSG1 | SEQ ID NO: 2 | MAAALFVLLG FALLGTHGAS GAAGFVQAPL |
| (isoform 1, long) | | SQQRWVGGSV ELHCEAVGSP VPEIQWWFEG |
| | | QGPNDTCSQL WDGARLDRVH IHATYHQHAA |
| | | STISIDTLVE EDTGTYECRA SNDPDRNHLT |
| | | RAPRVKWVRA QAVVLVLEPG TVFTTVEDLG |
| | | SKILLTCSLN DSATEVTGHR WLKGGVVLKE |
| | | DALPGQKTEF KVDSDDQWGE YSCVFLPEPM |
| | | GTANIQLHGP PRVKAVKSSE HINEGETAML |
| | | VCKSESVPPV TDWAWYKITD SEDKALMNGS |
| | | ESRFFVSSSQ GRSELHIENL NMEADPGQYR |
| | | CNGTSSKGSD QAIITLRVRS HLAALWPFLG |
| | | IVAEVLVLVT IIFIYEKRRK PEDVLDDDDA |
| | | GSAPLKSSGQ HQNDKGKNVR |
| | | QRNSS |
| BSG | SEQ ID NO: 36 | MKQSDASPQERVDSDDQWGEYSCVFLPEPMG |
| (isoform 4) | | TANIQLHGPPRVKAVKSSEHINEGETAMLVCK |
| | | SESVPPV |
| | | TDWAWYKITDSEDKALMNGSESRFFVSSSQGR |
| | | SELHIENLNMEADPGQYRCNGTSSKGSDQAIIT |
| | | LRVRS |
| | | HLAALWPFLGIVAEVLVLVTIIFIYEKRRKPED |
| | | VLDDDDAGSAPLKSSGQHQNDKGKNVRQRNS |
| | | S |

BSG2 is best known for its ability to induce extracellular matrix metalloproteinase and, therefore, has acquired the alternative name, “EMMPRIN.” BSG2 has also been shown to regulate lymphocyte responsiveness, monocarboxylate transporter expression, and spermatogenesis. These functions reflect the multiple interacting partners of BSG2. For example, interaction of BSG2 with proteins of the cyclophilin family has shown BSG2 to be a signalling receptor to extracellular cyclophilins A and B which are potent chemotactic agents for various immune cells. Further studies of the interactions between cyclophilins and BSG2 in inflammation have demonstrated that agents targeting BSG2 or cyclophilin had a significant anti-inflammatory effect in animal models of acute or chronic lung diseases and rheumatoid arthritis (V. Yurchenko et al. (2005) Immunol. 117(3):301-309). The various functions attributed to BSG2 have also spawned additional alternative names, such as “CD147,” “Leukocyte activation antigen M6,” “Collagenase stimulatory factor,” “5F7,” “Tumor cell-derived collagenase stimulatory factor (TCSF),” “OK blood group antigen,” “OX-47,” “Neurothelin,” “M6 antigen,” and “HT7 antigen (see, e.g., Miyauchi T. et al. (1990) J. Biochem., 107: 316-323; Fossum S. Et al. (1991) Eur. J. Immunol., 21: 671-679; Schlosshauer B. et al. (1995) Eur. J. Cell Biol., 68: 159-166; Kasinrerk W. et al. (1992) J. Immunol., 149: 847-854; Seulberger H. et al. (1990) EMBO J., 9: 2151-2158), all of which are used interchangeably herein and refer to to variants or isoforms of BSG2 which are naturally expressed by cells (e.g., human BSG2 (hBSG2) or mouse BSG2 (mBSG2)). Accordingly, antibodies of the invention may cross-react with BSG2 from species other than human. Alternatively, the antibodies may be specific for human BSG2 and may not exhibit any cross-reactivity with other species. BSG2 or any variants and isoforms thereof, may either be isolated from cells or tissues which naturally express them (e.g. human, mouse and cynomologous monkey cells) or be recombinantly produced using well-known techniques in the art and/or those described herein. The amino acid sequence of human BSG2 and the amino acid sequence of its related isoform are shown in Table 1.

“Biological activity,” as used herein, refers to all inherent biological properties of BSG2. Biological properties of BSG2 include but are not limited to the production or release of matrix metalloproteinases in the surrounding mesenchymal cells and tumor cells, thereby contributing to tumor invasion.

The terms “specific binding” or “specifically binding,” as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody,” as used herein, broadly refers to any immunoglobulin (Ig) molecule, or antigen-binding portion thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative anitbody formats are known in the art. Nonlimiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” or “antigen-binding region” of an antibody (or simply “antibody portion”), as used herein, refers to one or more fragments of an antibody that retain the ability to specifically bind to an antigen (e.g., hBSG2). The antigen-binding function of an antibody can be performed by fragments of a full-length antibody. Such antibody embodiments may also have bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct,” as used herein, refers to a polypeptide comprising one or more antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art and represented in Table 2.

TABLE 2
Sequence of human IgG heavy chain constant domain and light chain
constant domain

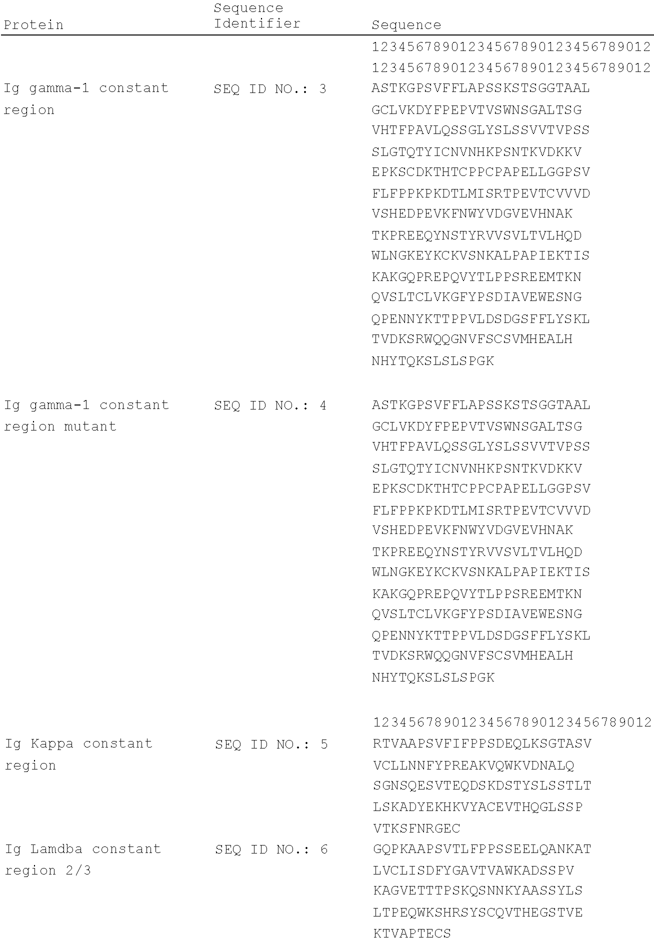

An antibody, or antigen-binding portion thereof, may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody,” as used herein, refers to an antibody, or antigen-binding portion thereof, that is substantially free of other antibodies having different antigenic specificities (e.g., an isolated antibody that specifically binds hBSG2 is substantially free of antibodies that specifically bind antigens other than hBSG2). An isolated antibody that specifically binds hBSG2 may, however, have cross-reactivity to other antigens, such as BSG2 molecules from other species. Moreover, an isolated antibody may be substantially free of other cellular material and/or chemicals.

The term “human antibody,” as used herein, is intended to include antibodies, or antigen-binding portions thereof, having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies, or antigen-binding portions thereof, that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom H.R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies, or antigen-binding portions thereof, which comprise heavy and light chain variable region sequences from one species and constant region sequences from another species, such as antibodies having murine heavy and light chain variable regions linked to human constant regions.

The term “CDR-grafted antibody” refers to antibodies, or antigen-binding portions thereof, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies, or antigen-binding portions thereof, which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

The terms “Kabat numbering,” “Kabat definitions,” and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia &Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although particular embodiments use Kabat or Chothia defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992)). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In a particular embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, —H2, and —H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art. In one embodiment of the invention the human heavy chain and light chain acceptor sequences are selected from the sequences listed from V-base (http://vbase.mrc-cpe.cam.ac.uk/) or from IMGT®, the international ImMunoGeneTics information system® (http://imgt.cines.fr/textes/IMGTrepertoire/LocusGenes/). In another embodiment of the invention the human heavy chain and light chain acceptor sequences are selected from the sequences described in Table 3 and Table 4.

TABLE 3
HEAVY CHAIN ACCEPTOR SEQUENCES

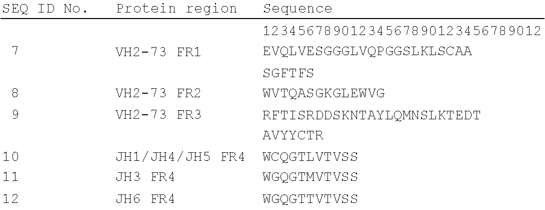

TABLE 4
LIGHT CHAIN ACCEPTOR SEQUENCES

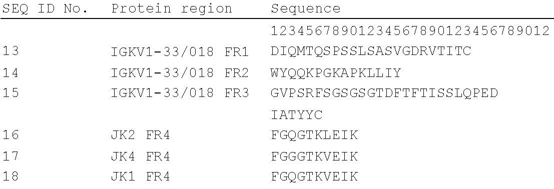

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol. 22(3): I83-200 (2002); Marchalonis et al., Adv Exp Med. Biol. 484:13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (e.g., N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

As used herein, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′) 2, FabC, Fv) in which all or substantially all of the CDR regions correspond to those of a non-human immunoglobulin (i.e., donor antibody) and all or substantially all of the framework regions are those of a human immunoglobulin consensus sequence. In a particular embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including, e.g., IgM, IgG, IgD, IgA and IgE, and any isotype, including without limitation, e.g., IgG 1, IgG2, IgG3 and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a particular embodiment, such mutations are not extensive. Usually, at least 80%, at least 85%, at least 90%, and at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499). Vernier zone residues form a layer underlying the CDRs and may impact on the structure of CDRs and the affinity of the antibody.

The term “multivalent binding protein” is used in this specification to denote a binding protein comprising two or more antigen binding sites. The multivalent binding protein may be engineered to have the three or more antigen binding sites, and is generally not a naturally occurring antibody. The term “multispecific binding protein” refers to a binding protein capable of binding two or more related or unrelated targets. Dual variable domain (DVD) binding proteins as used herein, are binding proteins that comprise two or more antigen binding sites and are tetravalent or multivalent binding proteins. Such DVDs may be monospecific, i.e capable of binding one antigen or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. DVD binding proteins and methods of making DVD binding proteins are disclosed in U.S. Pat. No. 7,612,181.

One aspect of the invention pertains to a DVD binding protein comprising binding proteins capable of binding BSG2. In a particular embodiment, the DVD binding protein is capable of binding BSG2 and a second target, e.g., an EGFR family member, cMet, VEGF, DLL4, or RON.

As used herein, the term “neutralizing” refers to neutralization of a biological activity of BSG2 when a binding protein specifically binds BSG2, e.g., hBSG2. In a particular embodiment, a neutralizing binding protein is a neutralizing antibody whose binding to BSG2 results in the inhibition of or a decrease in a biological activity of BSG2, e.g., cell signal transduction within the integrin pathway. In particular, the neutralizing binding protein binds BSG2 and reduces a biologically activity of BSG2 by at least about 20%, 40%, 60%, 80%, 85% or more Inhibition of a biological activity of BSG2 by a neutralizing binding protein can be assessed by measuring one or more indicators of hBSG2 biological activity well known in the art, for example, inhibition or blocking of the function of T effector cells induced by BSG2.

In another embodiment, as used herein, the term “agonizing” refers to an increase of a biological activity of BSG2 when a binding protein specifically binds BSG2, e.g., hBSG2. In a particular embodiment, an agonizing binding protein is an agonistic antibody whose binding to BSG2 results in the increase of a biological activity of BSG2, e.g., cell signal transduction. For example, the agonistic binding protein binds BSG2 and increases a biologically activity of BSG2 by at least about 20%, 40%, 60%, 80%, 85% or more. Increase of a biological activity of BSG2 by an agonistic binding protein can be assessed by measuring one or more indicators of hBSG2 biological activity well known in the art, for example, an increase in the activation of MMP, VEGF, or integrin signaling.

The term “activity” includes activities such as the binding specificity/affinity of an antibody for an antigen, for example, an anti-hBSG2 antibody that binds to an BSG2 antigen and/or the neutralizing potency (or agonizing potency) of an antibody, for example, an anti-hBSG2 antibody whose binding to hBSG2 inhibits the biological activity of hBSG2, e.g. inhibition of cell signal transduction and resulting cell death.

The term “epitope” or “antigenic determinant” refers to a site on an antigen to which an immunoglobulin or antibody specifically binds. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from BSG2 are tested for reactivity with the given anti-BSG2 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also, encompassed by the present invention are antibodies that bind to an epitope on BSG2 which comprises all or a portion of an epitope recognized by the particular antibodies described herein (e.g., the same or an overlapping region or a region between or spanning the region).

Also encompassed by the present invention are antibodies that bind the same epitope and/or antibodies that compete for binding to BSG2, e.g., human BSG2, with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as hBSG2. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “Kon”, as used herein, is intended to refer to the on rate constant for association of a binding protein (e.g., an antibody) to the antigen to form the, e.g., antibody/antigen complex as is known in the art. The “Kon” also is known by the terms “association rate constant”, or “ka”, as used interchangeably herein. This value indicating the binding rate of an antibody to its target antigen or the rate of complex formation between an antibody and antigen also is shown by the equation below:

Antibody (“Ab”)+Antigen (“Ag”)→Ab−Ag

The term “Koff”, as used herein, is intended to refer to the off rate constant for dissociation, or “dissociation rate constant”, of a binding protein (e.g., an antibody) from the, e.g., antibody/antigen complex as is known in the art. This value indicates the dissociation rate of an antibody from its target antigen or separation of Ab−Ag complex over time into free antibody and antigen as shown by the equation below:

Ab+Ag←Ab−Ag

The term “KD” as used herein, is intended to refer to the “equilibrium dissociation constant”, and refers to the value obtained in a titration measurement at equilibrium, or by dividing the dissociation rate constant (koff) by the association rate constant (kon). The association rate constant, the dissociation rate constant and the equilibrium dissociation constant are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.) can also be used.

The term “labeled binding protein” as used herein, refers to a protein with a label incorporated that provides for the identification of the binding protein. In a particular embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, 166Ho, or ¹⁵³Sm); fluorescent labels (e.g., FITC, rhodamine, lanthanide phosphors, europium), enzymatic labels (e.g., horseradish peroxidase, luciferase, alkaline phosphatase); chemiluminescent markers; electrochemiluminescent labels (MesoScale Electrochemiluminescent Technology, MSD, Gaithersburg, Md.) biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody conjugate” refers to a binding protein, such as an antibody, chemically linked to a second chemical moiety, such as a therapeutic or cytotoxic agent. The term “agent” is used herein to denote a chemical compound, a mixture of chemical compounds, a biological macromolecule, or an extract made from biological materials. In a particular embodiment, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

The terms “crystal” and “crystallized,” as used herein, refer to an antibody, or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N.Y., (1999).”

The term “polynucleotide” as referred to herein means a polymeric form of two or more nucleotides, either ribonucleotides (RNAs) or deoxyribonucleotides (DNAs) or a modified form of either type of nucleotide. The term includes single and double stranded forms of DNA but in a particular embodiment is double-stranded DNA.

The term “isolated polynucleotide,” as used herein, refers to a polynucleotide (e.g., of genomic, cDNA, or synthetic origin, or some combination thereof) that, by virtue of its origin, the “isolated polynucleotide”: is not associated with all or a portion of a polynucleotide with which the “isolated polynucleotide” is found in nature; is operably linked to a polynucleotide that it is not linked to in nature; or does not occur in nature as part of a larger sequence.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest.

The term “expression control sequence,” as used herein, refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation,” as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. In a particular embodiment, host cells include prokaryotic and eukaryotic cells selected from any of the Kingdoms of life. Eukaryotic cells include protist, fungal, plant and animal cells. In a particular embodiment, host cells include but are not limited to the prokaryotic cell line E. Coli; mammalian cell lines CHO, HEK 293 and COS; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)).

“Transgenic organism,” as known in the art and as used herein, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The term “regulate” and “modulate” are used interchangeably, and, as used herein, refers to a change or an alteration in the activity of a molecule of interest (e.g., the biological activity of hBSG2). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of hBSG2). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in WO01/83525.

The term “agonist,” as used herein, refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, BSG2 polypeptides or polypeptides, nucleic acids, carbohydrates, or any other molecules that bind to BSG2.

The term “antagonist” or “inhibitor,” as used herein, refers to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of BSG2, e.g., hBSG2. Antagonists and inhibitors of hBSG2 may include, but are not limited to, proteins, nucleic acids, carbohydrates, or any other molecules, which bind to BSG2.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The term “sample,” as used herein, is used in its broadest sense. A “biological sample,” as used herein, includes, but is not limited to, any quantity of a substance from a living thing or formerly living thing. Such living things include, but are not limited to, humans, mice, rats, monkeys, dogs, rabbits and other animals. Such substances include, but are not limited to, blood, serum, urine, synovial fluid, cells, organs, tissues, bone marrow, lymph nodes and spleen.

I. Antibodies that Bind Human BSG2

One aspect of the present invention provides isolated murine monoclonal antibodies, or antigen-binding portions thereof, that bind to BSG2 with high affinity, a slow off rate and high neutralizing capacity. A second aspect of the invention provides chimeric antibodies that bind BSG2. A third aspect of the invention provides CDR grafted antibodies, or antigen-binding portions thereof, that bind BSG2. A fourth aspect of invention provides humanized antibodies, or antigen-binding portions thereof, that bind BSG2. In a particular embodiment, the antibodies, or portions thereof, are isolated antibodies. The antibodies of the invention modulate human BSG2 functions.

### A. Method of Making Anti BSG2 Antibodies

Antibodies of the present invention may be made by any of a number of techniques known in the art.

### 1. Anti-BSG2 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In one embodiment, the present invention provides methods of generating monoclonal antibodies as well as antibodies produced by the method comprising culturing a hybridoma cell secreting an antibody of the invention wherein, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. Briefly, mice can be immunized with a BSG2 antigen. In a particular embodiment, the BSG2 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. In a particular embodiment, if a polypeptide is being administered, the immunization schedule will involve two or more administrations of the polypeptide, spread out over several weeks.

After immunization of an animal with a BSG2 antigen, or cells expressing BSG, antibodies and/or antibody-producing cells may be obtained from the animal. An anti-BSG2 antibody-containing serum is obtained from the animal by bleeding or sacrificing the animal. The serum may be used as it is obtained from the animal, an immunoglobulin fraction may be obtained from the serum, or the anti-BSG2 antibodies may be purified from the serum. Serum or immunoglobulins obtained in this manner are polyclonal, thus having a heterogeneous array of properties.

Once an immune response is detected, e.g., antibodies specific for the antigen BSG2 are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding BSG2. Ascites fluid, which generally contains high levels of antibodies, can be generated by immunizing mice with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. See, e.g., Harlow and Lane, supra. In a particular embodiment, the myeloma cells do not secrete immunoglobulin polypeptides (a non-secretory cell line). After fusion and antibiotic selection, the hybridomas are screened using BSG2, or a portion thereof, or a cell expressing BSG2. In a particular embodiment, the initial screening is performed using an enzyme-linked immunoassay (ELISA) or a radioimmunoassay (RIA), preferably an ELISA. An example of ELISA screening is provided in WO 00/37504.

Anti-BSG2 antibody-producing hybridomas are selected, cloned and further screened for desirable characteristics, including robust hybridoma growth, high antibody production and desirable antibody characteristics, as discussed further below. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a particular embodiment, the hybridomas are mouse hybridomas, as described above. In another embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-BSG2 antibody.

Antibody fragments that recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

### 2. Anti-BSG2 Monoclonal Antibodies Using Slam

In another aspect of the invention, recombinant antibodies are generated from single, isolated lymphocytes using a procedure referred to in the art as the selected lymphocyte antibody method (SLAM), as described in U.S. Pat. No. 5,627,052, PCT Publication WO 92/02551 and Babcock, J. S. et al. (1996) Proc. Natl. Acad. Sci. USA 93:7843-7848. In this method, single cells secreting antibodies of interest, e.g., lymphocytes derived from any one of the immunized animals described in Section 1, are screened using an antigen-specific hemolytic plaque assay, wherein the antigen BSG2, a subunit of BSG2, or a fragment thereof, is coupled to sheep red blood cells using a linker, such as biotin, and used to identify single cells that secrete antibodies with specificity for BSG2. Following identification of antibody-secreting cells of interest, heavy- and light-chain variable region cDNAs are rescued from the cells by reverse transcriptase-PCR and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example by panning the transfected cells to isolate cells expressing antibodies to BSG2. The amplified immunoglobulin sequences further can be manipulated in vitro, such as by in vitro affinity maturation methods such as those described in PCT Publication WO 97/29131 and PCT Publication WO 00/56772.

### 3. Anti-BSG2 Monoclonal Antibodies Using Transgenic Animals

In another embodiment of the instant invention, antibodies are produced by immunizing a non-human animal comprising some, or all, of the human immunoglobulin locus with a BSG2 antigen. In a particular embodiment, the non-human animal is a XENOMOUSE transgenic mouse, an engineered mouse strain that comprises large fragments of the human immunoglobulin loci and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The XENOMOUSE transgenic mouse produces an adult-like human repertoire of fully human antibodies, and generates antigen-specific human Mabs. The XENOMOUSE transgenic mouse contains approximately 80% of the human antibody repertoire through introduction of megabase sized, germline configuration YAC fragments of the human heavy chain loci and x light chain loci. See Mendez et al., Nature Genetics 15:146-156 (1997), Green and Jakobovits J. Exp. Med. 188:483-495 (1998).

### 4. Anti-BSG2 Monoclonal Antibodies Using Recombinant Antibody Libraries

In vitro methods also can be used to make the antibodies of the invention, wherein an antibody library is screened to identify an antibody having the desired binding specificity. Methods for such screening of recombinant antibody libraries are well known in the art and include methods described in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, US patent application publication 20030186374, and PCT Publication No. WO 97/29131.

The recombinant antibody library may be from a subject immunized with BSG2, or a portion of BSG2. Alternatively, the recombinant antibody library may be from a naïve subject, i.e., one who has not been immunized with BSG2, such as a human antibody library from a human subject who has not been immunized with human BSG2. Antibodies of the invention are selected by screening the recombinant antibody library with the peptide comprising human BSG2 to thereby select those antibodies that recognize BSG2. Methods for conducting such screening and selection are well known in the art, such as described in the references in the preceding paragraph. To select antibodies of the invention having particular binding affinities for hBSG2, such as those that dissociate from human BSG2 with a particular k_{off} rate constant, the art-known method of surface plasmon resonance can be used to select antibodies having the desired k_{off} rate constant. To select antibodies of the invention having a particular neutralizing activity for hBSG2, such as those with a particular an IC₅₀, standard methods known in the art for assessing the inhibition of hBSG2 activity may be used.

In one aspect, the invention pertains to an isolated antibody, or an antigen-binding portion thereof, that binds BSG2, e.g., human BSG2. In a particular embodiment, the antibody is a neutralizing antibody. In various embodiments, the antibody is a recombinant antibody or a monoclonal antibody.

For example, the antibodies of the present invention can also be generated using various phage display methods known in the art. In phage display methods, functional antibody domains are displayed on the surface of phage particles which carry the polynucleotide sequences encoding them. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

Alternative to screening of recombinant antibody libraries by phage display, other methodologies known in the art for screening large combinatorial libraries can be applied to the identification of dual specificity antibodies of the invention. One type of alternative expression system is one in which the recombinant antibody library is expressed as RNA-protein fusions, as described in PCT Publication No. WO 98/31700 by Szostak and Roberts, and in Roberts, R. W. and Szostak, J. W. (1997) Proc. Natl. Acad. Sci. USA 94:12297-12302. In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above.

In another approach the antibodies of the present invention can also be generated using yeast display methods known in the art. In yeast display methods, genetic methods are used to tether antibody domains to the yeast cell wall and display them on the surface of yeast. In particular, such yeast can be utilized to display antigen-binding domains expressed from a repertoire or combinatorial antibody library (e.g., human or murine). Examples of yeast display methods that can be used to make the antibodies of the present invention include those disclosed Wittrup, et al. U.S. Pat. No. 6,699,658.

### B. Production of Recombinant BSG2 Antibodies

Antibodies of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the invention in either prokaryotic or eukaryotic cells is contemplated, for example, in mammalian host cells, because such eukaryotic cells (and in particular mammalian cells) are more likely than prokaryotic cells to assemble and secrete a properly folded and immunologically active antibody.

Mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P.A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of an antibody of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the antigens of interest by crosslinking an antibody of the invention to a second antibody by standard chemical crosslinking methods.

In an exemplary system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the invention provides a method of synthesizing a recombinant antibody of the invention by culturing a host cell of the invention in a suitable culture medium until a recombinant antibody of the invention is synthesized. The method can further comprise isolating the recombinant antibody from the culture medium.

1. Anti hBSG2 Antibodies

The figures, examples, and sequence listing include a list of amino acid sequences of VH and VL regions (CDR sequences designated) of anti-hBSG2 antibodies of the invention. Tables 5 and 6 below provide exemplary murine antibodies 3A3 and 2C1, respectively.

TABLE 5
VH AND VL NUCLEOTIDE AND AMINO ACID SEQUENCES OF MOUSE
BSG2 MONOCLONAL ANTIBODY 3A3 (CDR SEQUENCES IN BOLD)

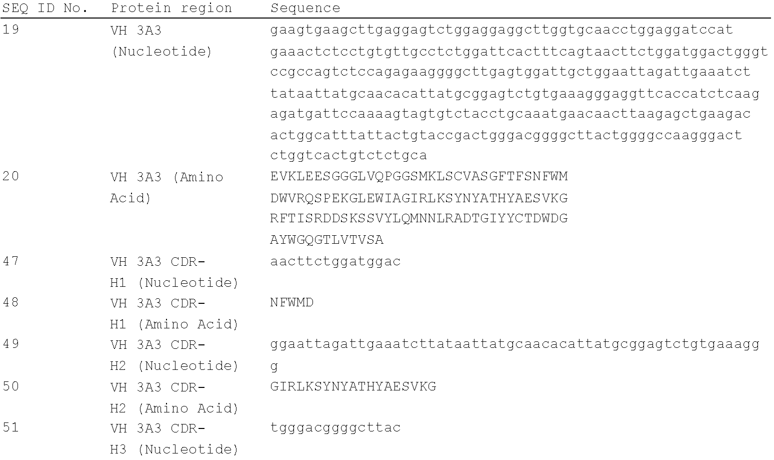

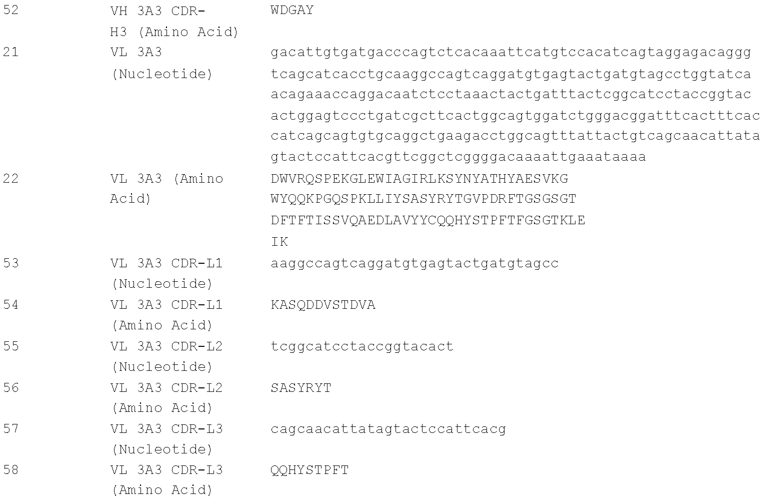

TABLE 6
VH AND VL NUCLEOTIDE AND AMINO ACID SEQUENCES OF MOUSE
BSG2 MONOCLONAL ANTIBODY 2C1 (CDR SEQUENCES IN BOLD)

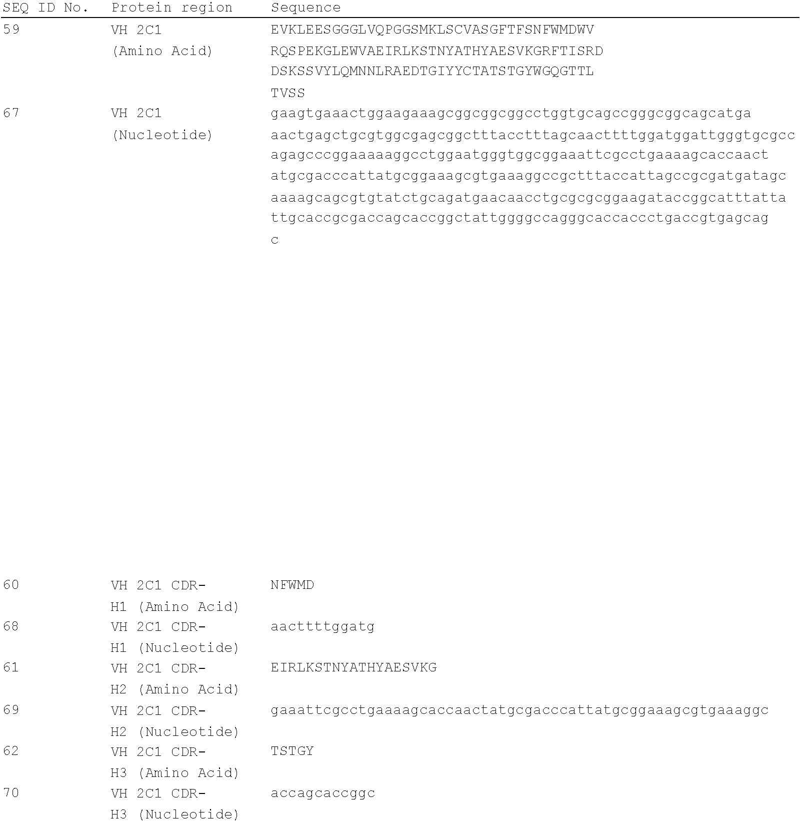

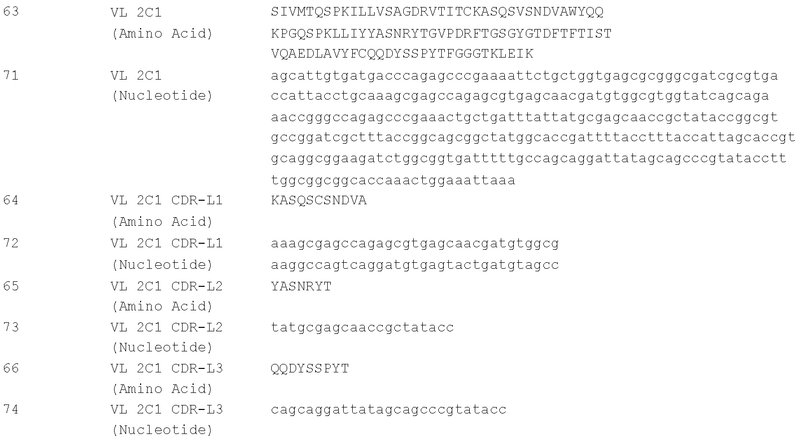

TABLE 7
VH AND VL AMINO ACID SEQUENCES OF MOUSE BSG2
MONOCLONAL ANTIBODY 2A1 (CDR SEQUENCES IN BOLD)

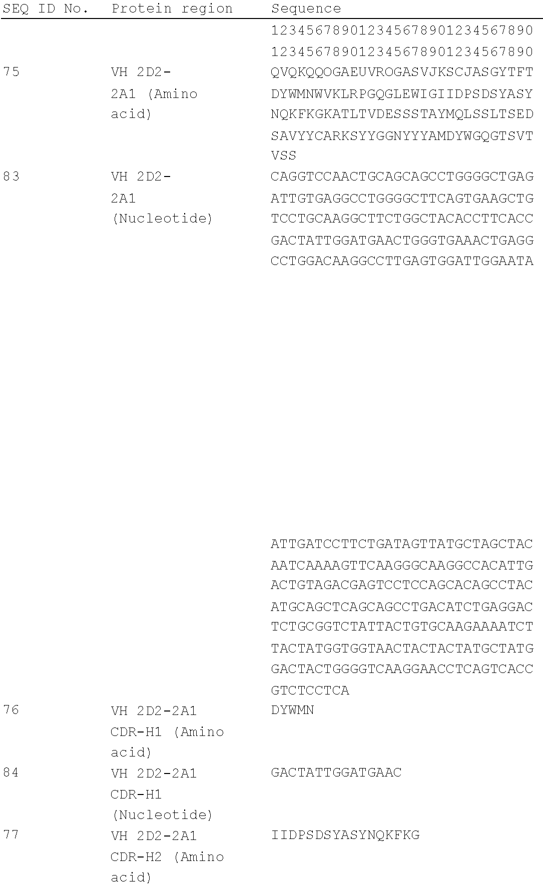

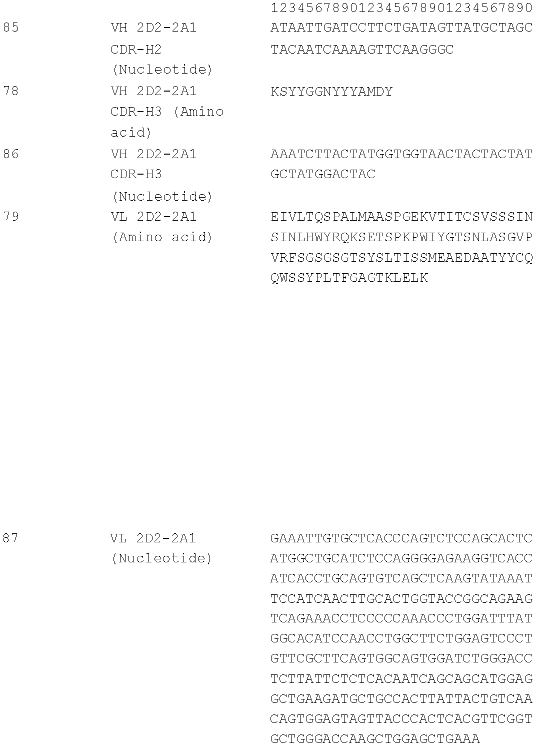

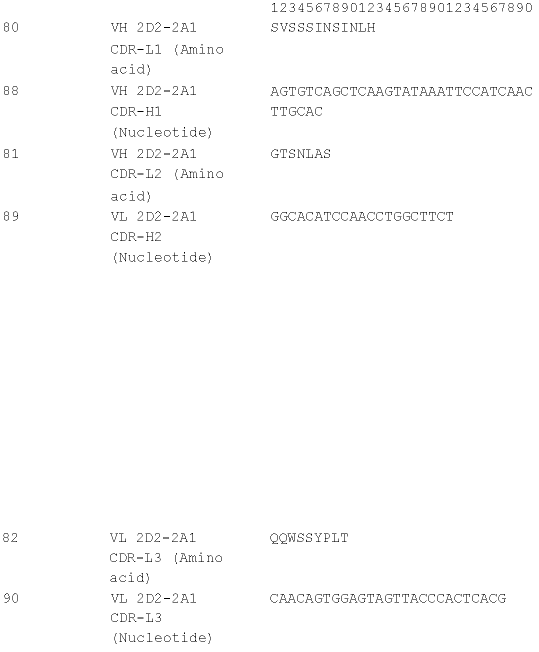

2. Anti hBSG2 Chimeric Antibodies

A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397. In addition, techniques developed for the production of “chimeric antibodies” (Morrison et al., 1984, Proc. Natl. to Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used.

In one embodiment, the chimeric antibodies of the invention are produced by replacing the heavy chain constant region of the murine monoclonal anti human BSG2 antibodies described in section 1 with a human IgG1 constant region.

### 3. Anti BSG2 CDR Grafted Antibodies

CDR-grafted antibodies of the invention comprise heavy and light chain variable region sequences from a human antibody wherein one or more of the CDR regions of V_{H} and/or V_{L} are replaced with CDR sequences of the murine antibodies of the invention. A framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, in a particular embodiment, the human variable framework that is chosen to replace the murine variable framework apart from the CDRs have at least a 65% sequence identity with the murine antibody variable region framework. In a particular embodiment, the human and murine variable regions apart from the CDRs have at least 70% sequence identify. In another embodiment, the human and murine variable regions apart from the CDRs have at least 75% sequence identity. In another embodiment, the human and murine variable regions apart from the CDRs have at least 80% sequence identity. Methods for producing chimeric antibodies are known in the art. (also see EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352).

### 4. Anti-hBSG2 Humanized Antibodies

Humanized antibodies are antibody molecules from non-human species antibody that binds the desired antigen having one or more complementarity determining regions (CDRs) from the non-human species and framework regions from a human immunoglobulin molecule. Known human Ig sequences are disclosed, e.g., www.ncbi.nlm.nih.gov/entrez-/query.fcgi; www.atcc.org/phage/hdb.html; www.sciquest.com/; www.abcam.com/; www.antibodyresource.com/onlinecomp.html; www.public.iastate.edu/.about.pedro/research_tools.html; www.mgen.uni-heidelberg.de/SD/IT/IT.html; www.whfreeman.com/immunology/CH-05/kuby05.htm; www.library.thinkquest.org/12429/Immune/Antibody.html; www.hhmi.org/grants/lectures/1996/vlab/; www.path.cam.ac.uk/.about.mrc7/m-ikeimages.html; www.antibodyresource.com/; mcb.harvard.edu/BioLinks/Immuno-logy.html.www.immunologylink.com/; pathbox.wustl.edu/.about.hcenter/index.-html; www.biotech.ufl.edu/.about.hcl/; www.pebio.com/pa/340913/340913.html-; www.nal.usda.gov/awic/pubs/antibody/; www.m.ehime-u.acjp/.about.yasuhito-/Elisa.html; www.biodesign.com/table.asp; www.icnet.uk/axp/facs/davies/lin-ks.html; www.biotech.ufl.edu/.about.fccl/protocol.html; www.isac-net.org/sites_geo.html; aximtl.imt.uni-marburg.de/.about.rek/AEP-Start.html; baserv.uci.kun.n1/.aboutjraats/linksl.html; www.recab.uni-hd.de/immuno.bme.nwu.edu/; www.mrc-cpe.cam.ac.uk/imt-doc/pu-blic/INTRO.html; www.ibt.unam.mx/vir/V_mice.html; imgt.cnusc.fr:8104/; www.biochem.ucl.ac.uk/.about.martin/abs/index.html; antibody.bath.ac.uk/; abgen.cvm.tamu.edu/lab/wwwabgen.html; www.unizh.ch/.about.honegger/AHOsem-inar/Slide01.html; www.cryst.bbk.ac.uk/.about.ubcg07s/; www.nimr mrc.ac.uk/CC/ccaewg/ccaewg.htm; www.path.cam.ac.uk/.about.mrc7/h-umanisation/TAHHP.html; www.ibt.unam.mx/vir/structure/stat_aim.html; www.biosci.missouri.edu/smithgp/index.html; www.cryst.bioc.cam.ac.uk/.abo-ut.fmolina/Web-pages/Pept/spottech.html; wwwjerini.de/fr roducts.htm; www.patents.ibm.com/ibm.html.Kabat et al., Sequences of Proteins of Immunological Interest, U.S. Dept. Health (1983). Such imported sequences can be used to reduce immunogenicity or reduce, enhance or modify binding, affinity, on-rate, off-rate, avidity, specificity, half-life, or any other suitable characteristic, as known in the art.

Framework residues in the human framework regions may be substituted with the corresponding residue from the CDR donor antibody to alter, improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; Riechmann et al., Nature 332:323 (1988)). Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Antibodies can be humanized using a variety of techniques known in the art, such as but not limited to those described in Jones et al., Nature 321:522 (1986); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), Padlan, Molecular Immunology 28(4/5):489-498 (1991); Studnicka et al., Protein Engineering 7(6):805-814 (1994); Roguska. et al., PNAS 91:969-973 (1994); PCT publication WO 91/09967, PCT/: US98/16280, US96/18978, US91/09630, US91/05939, US94/01234, GB89/01334, GB91/01134, GB92/01755; WO90/14443, WO90/14424, WO90/14430, EP 229246, EP 592,106; EP 519,596, EP 239,400, U.S. Pat. Nos. 5,565,332, 5,723,323, 5,976,862, 5,824,514, 5,817,483, 5,814,476, 5,763,192, 5,723,323, 5,766886, 5,714,352, 6,204,023, 6,180,370, 5,693,762, 5,530,101, 5,585,089, 5,225,539; 4,816,567.

Example 6 below describes production of exemplary humanized antibodies that bind BSG2. The humanized antibody herein comprises CDRs from a monoclonal murine antibody 3A3 incorporated into human variable heavy and light domains with appropriate framework and back mutations.

In a particular embodiment, CDR1 (NFWMD, i.e., SEQ ID NO:48); CDR2 (GIRLKSYNYATHYAESVKG, i.e., SEQ ID NO:50) and CDR3 (WDGAY, i.e., SEQ ID NO:52) from the variable heavy chain of 3A3 may be grafted into VH3-73 as the human acceptor sequence. Additionally, in various embodiments, hJH4 may be used as the FR4 sequence.

Where VH3-73 is used as the acceptor sequence, further framework consensus or back mutations may be made as follows: V48I, G49A, N76S, A78V, R94A, R94D, K19R, S41P, K83R and/or T84A. Accordingly, in particular embodiments of the present invention, the humanized antibody of the present invention comprises a heavy chain as follows:

h3A3VH.1z (SEQ ID NO:26) is a CDR-grafted humanized 3A3 VH containing VH3-73 and hJH4 framework sequences.

h3A3VH.1 (SEQ ID NO:27) is a humanized design incorporating K19R, S41P, K83R, and T84A VH3 framework consensus changes.

h3A3VH.1a (SEQ ID NO:28) is a humanized design containing the consensus changes and all possible framework backmutations below.

Alternatively or in addition, CDR1 (KASQDVSTDVA, i.e., SEQ ID NO:54); CDR2 (SASYRYT, i.e., SEQ ID NO:56) and CDR3 (QQHYSTPFT, i.e., SEQ ID NO:58) from the variable light chain of 3A3 may be grafted into O8/O18 as the human light chain acceptor sequence. Additionally, in various embodiments, hJk2 may be used as the FR4 sequence.

Where O8/O18 is used as the acceptor light chain sequence, further framework consensus or back mutations may be made as follows: Q3V, I83F and/or A43S. Accordingly, in particular embodiments of the present invention, the humanized antibody comprises a humanized light chain as follows:

h3A3VL.1z (SEQ ID NO:32) is a direct CDR-grafted humanized 3A3 VL containing O18 and Jk2 framework sequences.

h3A3VL.1 (SEQ ID NO:33) is a humanized design incorporating I83F Vk1 framework consensus change.

h3A3VL.1a (SEQ ID NO:34) is a humanized design containing the consensus change and one possible framework backmutation (A43S).

h3A3VL.1b (SEQ ID NO:35) is a humanized design containing the consensus change and two framework back-mutations.

Example 7 below describes production of exemplary humanized antibodies that bind BSG2. The humanized antibody herein comprises CDRs from a monoclonal murine antibody 2C1 incorporated into human variable heavy and light domains with appropriate framework and back mutations.

In a particular embodiment, CDR1 (NFWMD, i.e., SEQ ID NO:60); CDR2 (EIRLKSTNYATHYAESVKG, i.e., SEQ ID NO:61) and CDR3 (TSTGY, i.e., SEQ ID NO:62) from the variable heavy chain of 2C1 may be grafted into VH3-73 as the human acceptor sequence. Additionally, in various embodiments, hJH6 may be used as the FR4 sequence.

Where VH3-73 is used as the acceptor sequence, further framework consensus and back mutations may be made as follows: G49A, N76S, A78V and/or R94A. Accordingly, in particular embodiments of the present invention, the humanized antibody of the present invention comprises a heavy chain as follows:

VH3-73JH6.5 (SEQ ID NO:37) is a fully human VH with only germline residues from VH3-73 and JH6 separated by a 5 A.A. CDR3.

h2C1VH.1 (SEQ ID NO:38) is a CDR grafted humanized 2C1 VH containing VH3-73 and JH6 framework sequences.

h2C1VH.1a (SEQ ID NO:39) is a humanized design based on 0.1 and contains 4 proposed framework consensus or back mutations G49A, N76S, A78V and R94A.

h2C1VH.1b (SEQ ID NO:40) is a compromised design between 0.1 and 0.1a containing one R94A back mutation.

Alternatively or in addition, CDR1 (KASQSVSNDVA, i.e., SEQ ID NO:64); CDR2 (YASNRYT, i.e., SEQ ID NO:65) and CDR3 (QQDYSSPYT, i.e., SEQ ID NO:66) from the variable light chain of 2C1 may be grafted into either O8/O18 or 3-15/L2 as the human light chain acceptor sequence. Additionally, in various embodiments, hJk4 may be used as the FR4 sequence.

Where O8/O18 is used as the acceptor light chain sequence, further framework consensus and back mutations may be made as follows: A43S and/or Y87F. Accordingly, in particular embodiments of the present invention, the humanized antibody comprises a humanized light chain as follows:

h2C1VL.1 (SEQ ID NO:42) is a CDR-grafted humanized 2C1 VL containing O18 and Jk4 framework sequences.

H2C1VL.1a (SEQ ID NO:43) is a humanized design containing 2 proposed framework consensus or back-mutations A43S and Y87F.

Where 3-15/L2 is used as the acceptor light chain sequence, further framework consensus or back mutations may be made as follows: A43S, I58V and/or Y87F. Accordingly, in particular embodiments of the present invention, the humanized antibody comprises a humanized light chain as follows:

h2C1VL.2 (SEQ ID NO:45) is a direct CDR-grafted humanized 2C1 VL containing 3-15/L2 and Jk4 framework sequences.

H2C1VL.2a (SEQ ID NO:46) is a humanized design based on 0.2 and contains 3 framework consensus or back-mutations (A43S, I58V, and Y87F).

Additionally, humanized versions of the 2A1 antibody (i.e., with the variable heavy chain as set forth in SEQ ID NO:75 and the variable light chain as set forth in SEQ ID NO:76) may be generated in accordance with the present invention. For example, techniques as utilized in Example 6 for the generation of humanized versions of the 3A3 antibody or as utilized in Example 7 for the generation of humanized versions of the 2C1 antibody may be employed to generate humanized versions of the 2A1 antibody.

### C. Production of Antibodies and Antibody-Producing Cell Lines

In one embodiment, anti-BSG2 antibodies of the present invention, exhibit a high capacity to reduce /neutralize BSG2 activity, e.g., as assessed by any one of several in vitro and in vivo assays known in the art. Alternatively, anti-BSG2 antibodies of the present invention, also exhibit a high capacity to increase/agonize BSG2 activity

In particular embodiments, the isolated antibody, or antigen-binding portion thereof, binds human BSG2, wherein the antibody, or antigen-binding portion thereof, dissociates from human BSG2 with a k_{off} rate constant of about 0.1 s⁻¹ or less, as determined by surface plasmon resonance, or which inhibits human BSG2 activity with an IC₅₀ of about 1×10⁻⁶M or less. Alternatively, the antibody, or an antigen-binding portion thereof, may dissociate from human BSG2 with a k_{off} rate constant of about 1×10⁻²s⁻¹ or less, as determined by surface plasmon resonance, or may inhibit human BSG2 activity with an IC₅₀ of about 1×10⁻⁷M or less. Alternatively, the antibody, or an antigen-binding portion thereof, may dissociate from human BSG2 with a k_{off} rate constant of about 1×10⁻³s⁻¹ or less, as determined by surface plasmon resonance, or may inhibit human BSG2 activity with an IC₅₀ of about 1×10⁻⁸M or less. Alternatively, the antibody, or an antigen-binding portion thereof, may dissociate from human BSG2 with a k_{off} rate constant of about 1×10⁻⁴s⁻¹ or less, as determined by surface plasmon resonance, or may inhibit human BSG2 activity with an IC₅₀ of about 1×10⁻⁹M or less. Alternatively, the antibody, or an antigen-binding portion thereof, may dissociate from human BSG2 with a k_{off} rate constant of about 1×10⁻⁵s⁻¹ or less, as determined by surface plasmon resonance, or may inhibit human BSG2 activity with an IC₅₀ of about 1×10⁻¹⁰M or less. Alternatively, the antibody, or an antigen-binding portion thereof, may dissociate from human BSG2 with a k_{off} rate constant of about 1×10⁻⁵s⁻¹ or less, as determined by surface plasmon resonance, or may inhibit human BSG2 activity with an IC₅₀ of about 1×10⁻¹¹M or less.

In certain embodiments, the antibody comprises a heavy chain constant region, such as an IgG1, IgG2, IgG3, IgG4, IgA, IgE, IgM or IgD constant region. In one embodiment, the heavy chain constant region is an IgG1 heavy chain constant region or an IgG4 heavy chain constant region. Furthermore, the antibody can comprise a light chain constant region, either a kappa light chain constant region or a lambda light chain constant region. In another embodiment, the antibody comprises a kappa light chain constant region. Alternatively, the antibody portion can be, for example, a Fab fragment or a single chain Fv fragment.

Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for a therapeutic antibody. Certain human IgG isotypes, particularly IgG1 and IgG3, mediate ADCC and CDC via binding to FcγR5 and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment provides a labeled binding protein wherein an antibody or antibody portion of the invention is derivatized or linked to another functional molecule (e.g., another peptide or protein). For example, a labeled binding protein of the invention can be derived by functionally linking an antibody or antibody portion of the invention (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

Another embodiment of the invention provides a crystallized binding protein. In particular, the invention relates to crystals of whole anti-BSG2 antibodies and fragments thereof as disclosed herein, and formulations and compositions comprising such crystals. In one embodiment the crystallized binding protein has a greater half-life in vivo than the soluble counterpart of the binding protein. In another embodiment the binding protein retains biological activity after crystallization.

Crystallized binding protein of the invention may be produced according methods known in the art and as disclosed in WO 02072636.

Another embodiment of the invention provides a glycosylated binding protein wherein the antibody or antigen-binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Protein glycosylation depends on the amino acid sequence of the protein of interest, as well as the host cell in which the protein is expressed. Different organisms may produce different glycosylation enzymes (eg., glycosyltransferases and glycosidases), and have different substrates (nucleotide sugars) available. Due to such factors, protein glycosylation pattern, and composition of glycosyl residues, may differ depending on the host system in which the particular protein is expressed. Glycosyl residues useful in the invention may include, but are not limited to, glucose, galactose, mannose, fucose, n-acetylglucosamine and sialic acid. In one embodiment, the glycosylated binding protein comprises glycosyl residues such that the glycosylation pattern is human.

It is known to those skilled in the art that differing protein glycosylation may result in differing protein characteristics. For instance, the efficacy of a therapeutic protein produced in a microorganism host, such as yeast, and glycosylated utilizing the yeast endogenous pathway may be reduced compared to that of the same protein expressed in a mammalian cell, such as a CHO cell line. Such glycoproteins may also be immunogenic in humans and show reduced half-life in vivo after administration. Specific receptors in humans and other animals may recognize specific glycosyl residues and promote the rapid clearance of the protein from the bloodstream. Other adverse effects may include changes in protein folding, solubility, susceptibility to proteases, trafficking, transport, compartmentalization, secretion, recognition by other proteins or factors, antigenicity, or allergenicity. Accordingly, a practitioner may prefer a therapeutic protein with a specific composition and pattern of glycosylation, for example glycosylation composition and pattern identical, or at least similar, to that produced in human cells or in the species-specific cells of the intended subject animal.

Expressing glycosylated proteins different from that of a host cell may be achieved by genetically modifying the host cell to express heterologous glycosylation enzymes. Using techniques known in the art a practitioner may generate antibodies or antigen-binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. Pat. Nos. 7,449,308 and 7,029,872).

Further, it will be appreciated by one skilled in the art that a protein of interest may be expressed using a library of host cells genetically engineered to express various glycosylation enzymes, such that member host cells of the library produce the protein of interest with variant glycosylation patterns. A practitioner may then select and isolate the protein of interest with particular novel glycosylation patterns. In one embodiment, the protein having a particularly selected novel glycosylation pattern exhibits improved or altered biological properties.

### D. Uses of Anti-BSG2 Antibodies

Given their ability to bind to BSG2, e.g., human BSG2, the anti-human BSG2 antibodies, or portions thereof, of the invention can be used to detect BSG2 (e.g., in a biological sample, such as serum or plasma), using a conventional immunoassay, such as an enzyme linked immunosorbent assays (ELISA), an radioimmunoassay (RIA) or tissue immunohistochemistry. The invention provides a method for detecting BSG2 in a biological sample comprising contacting a biological sample with an antibody, or antibody portion, of the invention and detecting either the antibody (or antibody portion) bound to BSG2 or unbound antibody (or antibody portion), to thereby detect BSG2 in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Alternative to labeling the antibody, human BSG2 can be assayed in biological fluids by a competition immunoassay utilizing rhBSG2 standards labeled with a detectable substance and an unlabeled anti-human BSG2 antibody. In this assay, the biological sample, the labeled rhBSG2 standards and the anti-human BSG2 antibody are combined and the amount of labeled rhBSG2 standard bound to the unlabeled antibody is determined. The amount of human BSG2 in the biological sample is inversely proportional to the amount of labeled rhBSG2 standard bound to the anti-BSG2 antibody. Similarly, human BSG2 can also be assayed in biological fluids by a competition immunoassay utilizing rhBSG2 standards labeled with a detectable substance and an unlabeled anti-human BSG2 antibody.

In one embodiment, the antibodies and antibody portions of the invention are capable of neutralizing or agonizing BSG2 acitivity, e.g., human BSG2 activity, both in vitro and in vivo. Accordingly, such antibodies and antibody portions of the invention can be used to inhibit or increase hBSG2 activity, e.g., in a cell culture containing hBSG2, in human subjects or in other mammalian subjects having BSG2 with which an antibody of the invention cross-reacts. In one embodiment, the invention provides a method for inhibiting or increasing hBSG2 activity comprising contacting hBSG2 with an antibody or antibody portion of the invention such that hBSG2 activity is inhibited or increased. For example, in a cell culture containing, or suspected of containing hBSG2, an antibody or antibody portion of the invention can be added to the culture medium to inhibit or increase hBSG2 activity in the culture.

In another embodiment, the invention provides a method for reducing or increasing hBSG2 activity in a subject, advantageously from a subject suffering from a disease or disorder in which BSG2 activity is detrimental. The invention provides methods for reducing or increasing BSG2 activity in a subject suffering from such a disease or disorder, which method comprises administering to the subject an antibody or antibody portion of the invention such that BSG2 activity in the subject is reduced or increased. In a particular embodiment, the BSG2 is human BSG2, and the subject is a human subject. Alternatively, the subject can be a mammal expressing a BSG2 to which an antibody of the invention is capable of binding. Still further the subject can be a mammal into which BSG2 has been introduced (e.g., by administration of BSG2 or by expression of a BSG2 transgene). An antibody of the invention can be administered to a human subject for therapeutic purposes. Moreover, an antibody of the invention can be administered to a non-human mammal expressing a BSG2 with which the antibody is capable of binding for veterinary purposes or as an animal model of human disease. Regarding the latter, such animal models may be useful for evaluating the therapeutic efficacy of antibodies of the invention (e.g., testing of dosages and time courses of administration).

As used herein, the term “a disorder in which BSG2 activity is detrimental” is intended to include diseases and other disorders in which the presence of BSG2 activity in a subject suffering from the disorder has been shown to be or is suspected of being either responsible for the pathophysiology of the disorder or a factor that contributes to a worsening of the disorder. Accordingly, a disorder in which BSG2 activity is detrimental is a disorder in which reduction (or an increase) of BSG2 activity is expected to alleviate the symptoms and/or progression of the disorder. Such disorders may be evidenced, for example, by an increase in the concentration of BSG2 in a biological fluid of a subject suffering from the disorder (e.g., an increase in the concentration of BSG2 in serum, plasma, synovial fluid, etc. of the subject), which can be detected, for example, using an anti-BSG2 antibody as described above. Non-limiting examples of disorders that can be treated with the antibodies of the invention include those disorders discussed in the section below pertaining to pharmaceutical compositions of the antibodies of the invention.

### E. Pharmaceutical Compositions

The invention also provides pharmaceutical compositions comprising an antibody, or antigen-binding portion thereof, of the invention and a pharmaceutically acceptable carrier. The pharmaceutical compositions comprising antibodies of the invention are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies of the invention. In another embodiment, the pharmaceutical composition comprises one or more antibodies of the invention and one or more prophylactic or therapeutic agents other than antibodies of the invention for treating a disorder in which BSG2 activity is detrimental. In a particular embodiment, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it may be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

Various delivery systems are known and can be used to administer one or more antibodies of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidurala administration, intratumoral administration, and mucosal adminsitration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903. In one embodiment, an antibody of the invention, combination therapy, or a composition of the invention is administered using Alkermes AIR® pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent can be delivered in a controlled release or sustained release system. In one embodiment, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), polyethylene-co-vinyl acetate), poly(methacrylic acid), polyglycolides (PLG), polyanhydrides, poly(N-vinyl pyrrolidone), polyvinyl alcohol), polyacrylamide, polyethylene glycol), polylactides (PLA), poly(lactide-co-glycolides) (PLGA), and polyorthoesters. In a particular embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy &Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, optionally in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903. In a specific embodiment, an antibody of the invention, combination therapy, and/or composition of the invention is administered using Alkermes AIR® pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt). The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline.

Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. In another embodiment, one or more of the prophylactic or therapeutic agents or pharmaceutical compositions of the invention is supplied as a dry sterile lyophilized powder in a hermetically sealed container at a unit dosage of at least 5 mg, at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, e.g., within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. In another embodiment, the liquid form of the administered composition is supplied in a hermetically sealed container at least 0.25 mg/ml, at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The antibodies and antibody-portions of the invention can be incorporated into a pharmaceutical composition suitable for parenteral administration. In particular, the antibody or antibody-portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical compositions are in the form of injectable or infusible solutions, such as compositions similar to those used for passive immunization of humans with other antibodies. Mode of administration includes parenteral (e.g., intravenous, subcutaneous, intraperitoneal, intramuscular). In a particular embodiment, the antibody is administered by intravenous infusion or injection. In another embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, methods of preparation include vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies and antibody-portions of the present invention can be administered by a variety of methods known in the art, although for many therapeutic applications, for example, the route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is coformulated with and/or coadministered with one or more additional therapeutic agents that are useful for treating disorders in which BSG2 activity is detrimental. For example, an anti-hBSG2 antibody or antibody portion of the invention may be coformulated and/or coadministered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an antibody to BSG2 or fragment thereof is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044.

In a specific embodiment, nucleic acid sequences comprising nucleotide sequences encoding an antibody of the invention or another prophylactic or therapeutic agent of the invention are administered to treat, prevent, manage, or ameliorate a disorder or one or more symptoms thereof by way of gene therapy. Gene therapy refers to therapy performed by the administration to a subject of an expressed or expressible nucleic acid. In this embodiment of the invention, the nucleic acids produce their encoded antibody or prophylactic or therapeutic agent of the invention that mediates a prophylactic or therapeutic effect.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley &Sons, NY (1993); and Kriegler, Gene Transfer and Expression, A Laboratory Manual, Stockton Press, NY (1990). Detailed description of various methods of gene therapy are disclosed in US20050042664 A1.

BSG2 has been implicated in a variety of physiological and pathological activities, such as inducement of extracellular matrix metalloproteinase, regulation of lymphocyte responsiveness, monocarboxylate transporter expression, spermatogenesis, as well as immune and inflammatory regulation. Therefore, diseases which are encompassed by the present invention include, but are not limited to, Acquired Immunodeficiency Disease Syndrome; Acquired Immunodeficiency Related Diseases; acquired pernicious anaemia; Acute coronary syndromes; acute and chronic pain (different forms of pain); Acute Idiopathic Polyneuritis; acute immune disease associated with organ transplantation; acute or chronic immune disease associated with organ transplantation; Acute Inflammatory Demyelinating Polyradiculoneuropathy; Acute ischemia; acute liver disease; acute rheumatic fever; acute transverse myelitis; Addison's disease; adult (acute) respiratory distress syndrome; Adult Still's Disease; alcoholic cirrhosis; alcohol-induced liver injury; allergic diseases; allergy; alopecia; Alopecia greata; Alzheimer's disease; Anaphylaxis; ankylosing spondylitis; ankylosing spondylitis associated lung disease; Anti-Phospholipid Antibody Syndrome; Aplastic anemia; Arteriosclerosis; arthropathy; asthma; atheromatous disease/arteriosclerosis; atherosclerosis; atopic allergy; Atopic eczema; Atopic dermatitis; atrophic autoimmune hypothyroidism; autoimmune bullous disease; Autoimmune dermatitis; autoimmune diabetes; Autoimmune disorder associated with Streptococcus infection; Autoimmune Enteropathy; autoimmune haemolytic anaemia; autoimmune hepatitis; Autoimmune hearingloss; Autoimmune Lymphoproliferative Syndrome (ALPS); autoimmune mediated hypoglycaemia; Autoimmune myocarditis; autoimmune neutropenia; Autoimmune premature ovarian failure; autoimmune thrombocytopenia (AITP); autoimmune thyroid disease; autoimmune uveitis; bronchiolitis obliterans; Behcet's disease; Blepharitis; Bronchiectasis; Bullous pemphigoid; cachexia; Cardiovascular Disease; Catastrophic Antiphospholipid Syndrome; Celiac Disease; Cervical Spondylosis; chlamydia; choleosatatis; chronic active hepatitis; chronic eosinophilic pneumonia; chronic fatigue syndrome; chronic immune disease associated with organ transplantation; Chronic ischemia; chronic liver diseases; chronic mucocutaneous candidiasis; Cicatricial pemphigoid; Clinically isolated Syndrome (CIS) with Risk for Multiple Sclerosis; common varied immunodeficiency (common variable hypogammaglobulinaemia); connective tissue disease associated interstitial lung disease; Conjunctivitis; Coombs positive haemolytic anaemia; Childhood Onset Psychiatric Disorder; Chronic obstructive pulmonary disease (COPD); Crohn's disease; cryptogenic autoimmune hepatitis; cryptogenic fibrosing alveolitis; Dacryocystitis; depression; dermatitis scleroderma; dermatomyositis; dermatomyositis/polymyositis associated lung disease; Diabetic retinopathy; Diabetes mellitus; dilated cardiomyopathy; discoid lupus erythematosus; Disk herniation; Disk prolaps; disseminated intravascular coagulation; Drug-Induced hepatitis; drug-induced interstitial lung disease; Drug induced immune hemolytic anemia; Endocarditis; Endometriosis; endophthalmitis; enteropathic synovitis; Episcleritis; Erythema multiforme; erythema multiforme major; female infertility; fibrosis; fibrotic lung disease; Gestational pemphigoid; giant cell arteritis (GCA); glomerulonephritides; goitrous autoimmune hypothyroidism (Hashimoto's disease); Goodpasture's syndrome; gouty arthritis; graft versus host disease (GVHD); Grave's disease; group B streptococci (GBS) infection; Guillain-BarréSyndrome (GBS); haemosiderosis associated lung disease; Hay Fever; heart failure; hemolytic anemia; Henoch-Schoenlein purpurea; Hepatitis B; Hepatitis C; Hughes Syndrome; Huntington's chorea; hyperthyroidism; hypoparathyroidism; idiopathic leucopaenia; idiopathic thrombocytopaenia; Idiopathic Parkinson's Disease; idiopathic interstitial pneumonia; idiosyncratic liver disease; IgE-mediated Allergy; Immune hemolytic anemiae; Inclusion Body Myositise; infectious diseases; Infectious ocular inflammatory disease; inflammatory bowel disease; Inflammatory demyelinating disease; Inflammatory heart disease; Inflammatory kidney disease; insulin dependent diabetes mellitus; interstitial pneumonitis; IPF/UIP; Iritis; juvenile chronic arthritis; juvenile pernicious anaemia; Juvenile rheumatoid arthritis; Kawasaki's diseasee; Keratitis; Keratojuntivitis sicca; Kussmaul disease or Kussmaul-Meier Diseasee; Landry's Paralysis; Langerhan's Cell Histiocytosis; linear IgA disease; Livedo reticularis; Lyme arthritis; lymphocytic infiltrative lung disease; Macular Degeneration; male infertility idiopathic or NOS; malignancies; microscopic vasculitis of the kidneys; Microscopic Polyangiitis; mixed connective tissue disease associated lung disease; Morbus Bechterev; Motor Neuron Disorders; Mucous membrane pemphigoid; multiple sclerosis (all subtypes: primary progressive, secondary progressive, relapsing remitting etc.); Multiple Organ failure; myalgic encephalitis/Royal Free Disease; Myasthenia Gravis; Myelodysplastic Syndrome; myocardial infarction; Myocarditis; nephrotic syndrome; Nerve Root Disorders; Neuropathy; Non-alcoholic Steatohepatitis; Non-A Non-B Hepatitis; Optic Neuritis; organ transplant rejection; osteoarthritis; Osteolysis; Ovarian cancer; ovarian failure; Pancreatitis; Parasitic diseases; Parkinson's disease; Pauciarticular JRA; pemphigoid; pemphigus foliaceus; pemphigus vulgaris; peripheral artery occlusive disease (PAOD); peripheral vascular disease (PVD); peripheral artery disease (PAD); phacogenic uveitis; Phlebitis; Polyarteritis nodosa (or periarteritis nodosa); Polychondritis; Polymyalgia Rheumatica; Poliosis; Polyarticular JRA; Polyendocrine Deficiency Syndrome; Polymyositis; polyglandular deficiency type I and polyglandular deficiency type II; polymyalgia rheumatica (PMR); postinfectious interstitial lung disease; post-inflammatory interstitial lung disease; Post-Pump Syndrome; premature ovarian failure; primary biliary cirrhosis; primary myxoedema; primary parkinsonism; primary sclerosing cholangitis; primary sclerosing hepatitis; primary vasculitis; prostate and rectal cancer and hematopoietic malignancies (leukemia and lymphoma); Prostatitis; psoriasis; psoriasis type 1; psoriasis type 2; psoriatic arthritis; psoriatic arthropathy; pulmonary hypertension secondary to connective tissue disease; pulmonary manifestation of polyarteritis nodosa; Pure red cell aplasia; Primary Adrenal Insufficiency; radiation fibrosis; reactive arthritis; Reiter's disease; Recurrent Neuromyelitis Optica; renal disease NOS; Restenosis; rheumatoid arthritis; rheumatoid arthritis associated interstitial lung disease; Rheumatic heart disease; SAPHO (synovitis, acne, pustulosis, hyperostosis, and osteitis); sarcoidosis; Schizophreniae; Schmidt's syndrome; Scleroderma; Secondary Amyloidosis; Shock lung; Scleritis; Sciatica; Secondary Adrenal Insufficiency; sepsis syndrome; septic arthritis; septic shock; seronegative arthopathy; Silicone associated connective tissue disease;e Sjögren's disease associated lung disease; Sjörgren's syndrome; Sneddon-Wilkinson Dermatosis; sperm autoimmunity; spondyloarthropathy; spondilitis ankylosans; Stevens-Johnson Syndrome (SJS); Still's disease; stroke; sympathetic ophthalmia; Systemic inflammatory response syndrome; systemic lupus erythematosus; systemic lupus erythematosus associated lung disease; systemic sclerosis; systemic sclerosis associated interstitial lung disease; Takayasu's disease/arteritis; Temporal arteritis; Th2 Type and Th1 Type mediated diseases; thyroiditis; toxic shock syndrome; toxoplasmic retinitis; toxic epidermal necrolysis; Transverse myelitise; TRAPS (Tumor-necrosis factor receptor type 1 (TNFR)-Associated Periodic Syndrome); type B insulin resistance with acanthosis nigricans; Type 1 allergic reaction; type-1 autoimmune hepatitis (classical autoimmune or lupoid hepatitis); type-2 autoimmune hepatitis (anti-LKM antibody hepatitis)e; Type II Diabetes; ulcerative colitic arthropathye; ulcerative colitis; Urticaria; Usual interstitial pneumonia (UIP); uveitis; vasculitic diffuse lung disease; Vasculitis; Vernal conjunctivitis; viral retinitis; vitiligo; Vogt-Koyanagi-Harada syndrome (VKH syndrome); Wegener's granulomatosis; Wet macular degeneration; Wound healing; yersinia and salmonella associated arthropathy.

The antibodies, and antibody portions, of the invention can be used to treat humans suffering from a variety of tumorogenic diseases and disorders, e.g., by inhibiting tumor angiogenesis and/or tumor growth. Other diseases encompassed by the present invention include, for example, T-ALL (T-cell acute lymphoblastic leukemia), CADASIL (Cerebral Autosomal Dominant Arteriopathy with Sub-cortical Infarcts and Leukoencephalopathy), MS (Multiple Sclerosis), Tetralogy of Fallot, Alagille syndrome, basal cell carcinoma, acute T cell leukemia, primary and metastatic cancers, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), head and neck, and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), solid tumors arising from hematopoietic malignancies such as leukemias, and lymphomas (both Hodgkin's and non-Hodgkin's lymphomas). Preferably, the antibodies of the invention or antigen-binding portions thereof, are used to treat cancer or in the prevention of metastases from the tumors described above either when used alone or in combination with radiotherapy, other chemotherapeutic agents, and/or other biologic agents such as anti-cancer antibodies.

Antibodies of the invention, or antigen binding portions thereof, can be used alone or in combination to treat such diseases. It should be understood that the antibodies of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

The antibodies of the invention, or antigen binding portions thereof, may be combined with agents that include but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, angiogenesis inhibitors, anti-cancer biologics, anti-EGFR antibodies (e.g., cetuximab), anti-cMet antibodies, anti-ErbB3 antibodies, anti-HER2 antibodies (e.g., Herceptin), anti-VEGF antibodies (e.g., bevacizumab), anti-CD₂₀ antibodies, apoptosis inhibitors, and Bcl-2 family member inhibitors. The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1R1, sIL-1R11, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram. Particular combinations include methotrexate or leflunomide and in moderate or severe rheumatoid arthritis cases, cyclosporine.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### EXEMPLIFICATION

### Example 1

### In Vitro Assays Used to Characterize Anti-BSG2 Antibodies

Throughout the Examples, the following assays were used to identify and characterize anti-BSG2 antibodies unless otherwise stated.

### Example 1.1

### Binding of BSG2 Monoclonal Antibodies to Cell-Surface BSG2 as Assessed by Flow Cytometry (FACS)

Stable cell lines overexpressing cell-surface BSG2 or human tumor cell lines were harvested from tissue culture flasks, washed three times and resuspended in phosphate buffered saline (PBS) containing 1% bovine serum albumin and 1 mM CaCl₂ (FACS buffer). 5×10⁵ cells were incubated with antibodies at various concentrations in FACS buffer for 60 minutes on ice. Cells were washed twice and the bound murine monoclonal antibody was detected with a goat Fab′2 anti-mouse IgM+IgG+IgA (H+L) R-phycoerythrin-conjugated antibody (Southern Biotechnology Associates, Inc., Birmingham, Ala., Catalog No. 1012-08) diluted 1:500. Following incubation on ice (4° C., 60 minutes), cells were washed three times and resuspended in FACS buffer. Fluorescence was measured using a Becton Dickinson FACSCalibur-HTS (Becton Dickinson, San Jose, Calif., US). Data were analyzed using Graphpad Prism software and EC₅₀ values were reported as the concentration of antibody to achieve 50% of maximal BSG2 antibodies binding to BSG2 expressing cells.

### Example 1.2

### Affinity Determination Using BIACORE Surface Plasmon Resonance Technology

The BIACORE® surface plasmon resonance assay (Biacore, Inc., Piscataway, N.J., US) determines the affinity of antibodies with kinetic measurements of on-rate and off-rate constants. Binding of BSG2 antibodies to a purified recombinant BSG2 extracellular domain (ECD) was determined by surface plasmon resonance-based measurements with a Biacore® instrument (either a Biacore 2000, Biacore 3000, or Biacore T100; GE Healthcare, Piscataway, N.J., US). The assay format for mAb affinity analysis was Fc-based capture via immobilized anti-Fc IgG. A standard amine coupling protocol was employed to immobilize Fc-specific IgG via primary amines to the carboxy-methyl (CM) dextran surface of CMS sensorchips. For the study of mouse anti-human BSG2 mAbs, anti-mouse Fc was used as the immobilized capture reagent. An automated protocol, available on the Biacore 2000, was used to immobilize 8000-10000RU of capture reagent in all 4 flowcells of the sensorchip. Binding was recorded as a function of time and kinetic rate constants were calculated. In this assay, on-rates as fast as 10⁷M⁻¹ s⁻¹ and off-rates as slow as 10⁻⁵ s⁻¹ can be measured.

Briefly, the CM-dextran surfaces were activated by freshly prepared 1:1 50 mM N-hydroxysuccinimide (NHS):200 mM 3-(N,N-dimethylamino)propyl-N-ethylcarbodiimide (EDC). Then the anti-Fc IgG capture reagent (20 μg/ml in 10 mM sodium acetate, pH 4.5) was applied to the surface followed by deactivation of the surface and blocking of the residual reactive sites with 1M ethanolamine (pH 8.5). The running buffer employed was PBS-PB [1×PBS (Sigma P3813), pH 7.4, 0.005% P20 surfactant, 0.1 mg/ml BSA (Sigma-A7906)] and the assay temperature was 25° C. All reagents were diluted into running buffer to the specified concentrations. Each experimental cycle consisted of the following steps: (1) anti-BSG2 mAbs at 0.5 to 1 μg/ml were captured in flowcells 2, 3 or 4 to a level of 100-120RU. All measurements were referenced against flowcell 1 which had no captured anti-BSG2 mAb; (2) human BSG2 ECD was injected through all 4 flowcells, 240 μl at 80 μl/min. After the antigen injection, dissociation was monitored for 600 seconds at 80 μl/min; and (3) the anti-Fc capture surface was regenerated with 10 mM glycine, pH 1.5.

Rate constants were derived by making kinetic binding measurements at different antigen concentrations ranging from 1.23-900 nM, as a 3-fold dilution series, and included buffer-only injections (to be used for double referencing), and data were processed using Scrubber 2.0 software (BioLogic Software). The double referenced data from the BSG2 injection series for each antibody were then fit globally to the 1:1 (Langmuir) binding model, which included a floating bulkshift term, to determine the association and dissociation rate constants, kₒₙ (M⁻¹ s⁻¹) and k_{off} (s⁻¹). The equilibrium dissociation constant (K_{D} in units of M) is derived from the kinetic rate constants by the following relationship: K_{D}=k_{off}/kₒₙ.

### Example 1.3

### Binding of BSG2 Antibodies to Cell-Surface BSG2 as Determined by Electrochemiluminescence (ECL) Using Meso Scale Discovery (MSD) Technology

Cells were plated at 25,000 cells/well in 25 μl PBS using 96 well MSD high binding plates (MSD, catalogue no. L11XB-3), and incubated at 37° C. for 1 hour. 25 μl/well of blocking solution (30% serum in PBS) was added to the plates for 30 minutes at room temperature with mild agitation. Plates were then incubated with 25 μl/well of BSG2 antibodies (10 μg/ml) diluted in assay buffer (10% serum in PBS) for 1 hour at room temperature with mild agitation, and then washed 3× with PBS. Goat anti-mouse or anti-human IgG sulfotag was added at 1 μg/ml for 1 hour with mild agitation, and plates were washed 3× with PBS. After the final wash, plates were incubated with 2×MSD surfactant-free read buffer (catalogue no. R92TD-2) and the signal was detected on a MSD SECTOR Imager 6000. Data were analyzed using Graphpad Prism software and EC₅₀ values were reported.

### Example 1.4

### Binding of BSG2 Antibodies to Cell-Surface BSG2 as Determined by a Receptor Binding Assay (RBA)

Cell lines expressing BSG2 were harvested and washed once in assay buffer (PBS, 0.1% BSA, 0.02% Na Azide, 10 mM EDTA) and resuspended to 5×10⁶ cells per mL. Serial 1:2 dilutions of ⁺³Eu-labeled anti-BSG2 antibody stock in assay buffer were made for final concentrations ranging from 1.6×10⁻⁸-7.7×10⁻¹² M, and a 4 mg/mL solution of unlabeled anti-BSG2 antibody was also prepared. To 96 well round bottom plates, 25 μl of ⁺³Eu-labeled antibody dilutions were added to 25 μl buffer (total counts), or 25 μl of unlabeled antibody were added to 25 μl buffer (background counts). 50 μl of cells at 2.5×10⁵ cells per well were then added to all samples, mixed gently, and incubated on ice for 1 hour. Cell bound antibody was then separated from unbound antibody by transferring 20 μL of the reaction mixture to Acrowell 96 well filter plates (Pall PN 5020), centrifuged for 5 min (2200 rpm), washed 3× with 150 μL cold assay buffer, and centrifuged for 5 min. Signal was determined by incubating samples with 150 μL/well of enhancement buffer and read using Envision. The specific counts were determined by subtracting the background counts (cells+labeled antibody+excess cold antibody) from the corresponding total counts (cells+labeled antibody) for each antibody concentration. The labeled antibody concentration versus total counts was analyzed using Graphpad Prism 4 to calculate Kd and Bmax values.

### Example 1.5

### Induction of Complement-Dependent Cytotoxicity (CDC) by BSG2 Antibodies

The ability of BSG2 antibodies to promote cell killing of BSG2 expressing tumor cell lines was assessed using serum complement from rabbit (Harlan, Wis.). Briefly, human pancreatic carcinoma (MiaPaCa-2) and human hepatocellular carcinoma (HepG2) cells were resuspended in culture media at 1×10⁵ cells per ml. 100 μl of cell suspension was plated in 96-well microtiter plates overnight at 37° C. The media was removed and replaced with 100 μl of 1% BSA-PBS containing control or BSG2 antibodies, and incubated for 1 hour at 4° C. Plates were then washed with 1% BSA-PBS and 100 μl of DMEM media containing 10% or 20% of rabbit serum was added to each well. Plates were incubated for 4.5 hours at 37° C. For human lymphoma cells (DoHH-2), 20,000 cells in 50 μl of 1% BSA-PBS were plated in v-bottom 96-well microtiter plates. 50 μl of 2×1% BSA-PBS containing control or BSG2 antibodies were added for 1 hour at 4° C. Plates were then washed once with 1% BSA-PBS and resuspended in 100 μl DMEM containing 20% rabbit serum for 4.5 hours at 37° C. Viability of cells was assessed using CellTiter Glo (Promega, Wis.) according to the manufacturer's instructions.

### Example 1.6

### Induction of Antibody-Dependent Cellular Cytotoxicity (ADCC) by BSG2 Antibodies

The ADCC effector function of BSG2 antibodies on HepG2 cells was assessed using an M65 (CK18) ELISA kit (DiaPharma Group Inc., OH). PBMC (effector cells) and HepG2 (target cells) were collected and adjusted at 9×10⁶ cells/ml and 0.3×10⁶ cells/ml, respectively, to reach a 30:1 effector to target ratio. 50 μl PBMC and 50 μl of HepG2 cells were added to 100 μl of media containing various concentrations of different BSG2 antibodies. After 16 hours incubation at 37° C., cell-free supernatant was collected and subjected to M65 ELISA to measure EC₅₀ of percent specific lysis. Percent specific lysis=(sample CK18−spontaneous CK18)/(maximum CK18−spontaneous CK18)×100.

### Example 1.7

### Crosslinking of Cell-Surface BSG2 Using BSG2 Antibodies in Combination with a Secondary IgG Antibody Results in Inhibition of Akt Phosphorylation and Tumor Cell Growth

96 well microtiter plates were coated with 10 μg/ml of goat anti-mouse or goat anti-human IgG antibodies in PBS and incubated overnight at 4° C. Cells were washed in PBS and 2×10⁵ cells were incubated with BSG2 antibodies for 20 minutes on ice. Cells bound with BSG2 antibody were resuspended in 5 mls of culture media and 100 μl of sample was plated into secondary IgG-coated 96-well flat-bottom microtiter plates. For assessment of Akt phosphorylation at amino acid 473, plates were incubated at 37° C. for 1 hour and harvested for ph-Akt levels using a MSD® biomarker assay kit according to the manufacturer's instructions (Meso Scale Discovery, Gaithersburg, Md.). For cell viability assessment, plates were incubated at 37° C. for 3 days and then harvested using CellTiter Glo (Promega, Wis.) according to the manufacturer's instructions.

### Example 1.8

### Disruption of Mitochondrial Membrane Potential by BSG2 Antibodies

The ability of BSG2 antibodies to disrupt mitochondrial membrane potential was determined using the MitoProbe™ DiOC₂(3) dye (Invitrogen, catalogue no. M34150) that accumulates in healthy mitochondria with active membrane potentials. Briefly, microtiter plates were coated with 10 μg/ml of goat anti-mouse IgG antibody in PBS and incubated overnight at 4° C. Cells were washed in PBS and 2×10⁵ cells were incubated with BSG2 antibodies for 20 minutes on ice. Cells bound with BSG2 antibody were then resuspended in 5 mls of culture media and 100 μl of cell suspension was plated in coated 96-well flat-bottom microtiter plates and incubated at 37° C. for 24 hours. Plates were then washed with PBS (warmed at 37° C.) and cells were detached using trypsin-EDTA 0.25%, and resuspended in warm DMEM, and incubated in the presence of 50 nM of DiOC₂(3) at 37° C. for 30 minutes. Cells were then pelleted and resuspended in 500 ml of DMEM and stain intensity was analyzed on a flow cytometer with 488 nm excitation using a Becton Dickinson FACSCalibur-HTS (Becton Dickinson, San Jose, Calif., US). A decrease in DiOC₂(3) stain intensity is indicative of cell death.

### Example 2

### Generation and Isolation of Mouse Anti-Human BSG2 Monoclonal Antibodies by Hybridoma Technology

Stably human or cynomolgus monkey BSG2 transfected cells or human cells (293G-HEK) that endogenously express BSG2 were harvested from tissue culture, washed extensively with PBS and resuspended in PBS at a concentration of 2×10⁸ cells/ml, and injected into A/J and Balb/c mice (Jackson Labs) Animals were injected every three weeks for a total of 4 immunizations Animals used for fusions were given an additional boost of cells intraperitoneally four days prior to fusion. Spleen cells from immunized animals were fused with SP2/0-Ag-14 myeloma cells at a ratio of 5:1 using standard techniques of Kohler and Milstein (Kohler, G. and Milstein, C., (1975) Nature 256, 495-497). Seven to ten days post-fusion, when macroscopic colonies were observed in the fusion plate, supernatants were removed and tested by flow cytometry for specific binding to stably transfected cells expressing human or cynomolgus monkey BSG2. Specifically, approximately 1−5×10⁵ cells were incubated with hybridoma supernatant diluted 1:2-1:10 in PBS/FCS for 30-60 minutes on ice. Cells were washed twice and 100 μl of goat anti mouse IgG-Fc phycoerythrin (1:300 dilution in PBS/BSA) (Jackson ImmunoResearch, West Grove, Pa., Catalog No. 115-115-164) were added. After 30 minutes incubation on ice, cells were washed twice and resuspended in PBS/FCS. Fluorescence was measured using a Becton Dickinson FACSCalibur (Becton Dickinson, San Jose, Calif.). Hybridoma cells from FACS positive wells were scaled up and cloned by limiting dilution, and monoclonal antibodies from selected hybridomas were purified from tissue culture supernatants using Protein A chromatography. Table 8 represents three mouse anti-human BSG2 antibodies and their binding properties against human and cynomolgus monkey cell-surface BSG2, as reflective by EC₅₀ values.

TABLE 8
Binding of mouse anti-human BSG2 monoclonal antibodies to
cell-surface BSG2 as assessed by flow cytometry.

| | | FACS binding, EC₅₀ (nM) | | |
|---|---|---|---|---|
| | | BAF3- | | HepG2 |
| | | human | BAF3-cyno | (hepatocellular |
| Hybridoma ID | Isotype | BSG2 | BSG2 | carcinoma cells) |
| ML64-6A11-3A3 | IgG2a/κ | 0.41 | 0.35 | 0.32 |
| EB41-1F4-2C1 | IgG1/κ | 0.2 | 0.71 | 0.17 |
| SZ66-2D2-2A1 | IgG2a/κ | 0.14 | No binding | 0.17 |

### Example 3

### Deduction of Variable Region Protein Sequences of Anti-BSG2 Mouse Monoclonal Antibodies by DNA Cloning and Sequencing

The heavy- and light-chain variable region sequences corresponding to BSG2 hybridoma 2D2-2A1, 1F4-2C1, and 6A11-3A3 were determined by standard methodologies and are set forth in Table 9.

Total RNA was extracted from hybridoma frozen cell stocks by combining organic extraction and Qiagen's RNeasy midi kit (Qiagen, catalog #75144). 2 ml of frozen cells were resuspended in 8 ml of Qiazol (Qiagen, catalog #79306), vortexed briefly and incubated at room temperature for 5 minutes. Subsequently, 1.5 ml of chloroform was added to each sample and inverted vigorously 20 times. The aqueous layer (˜6 ml) was isolated by centrifuging the sample at 5000×G and combined with 6 ml of 70% ethanol (RNase free), which was mixed thoroughly by inversion. Total RNA was isolated by passing the resulting solution through the RNAeasy midi-column attached to a vacuum manifold, thus all flow through was discarded. Organic impurities were removed by washing the RNA bound to the RNaeasy midi-column by adding 4 ml of buffer RW1 (supplied by Qiagen) to the column.

This RWI wash was repeated one additional time. The RNA bound to the RNaeasy midi-column was washed again by adding 3 ml of RPE buffer (supplied by Qiagen), discarding flow through. The same step was repeated one more time, but after all visible liquid passed into the vacuum reservoir, the column was centrifuged for 3 minutes at 4500×G, which eliminated any carryover of buffer RPE. RNA was eluted with 200 μl of RNase-free Te-8 by centrifuging for 5 minutes at 4,500×G. The RNA quantity and quality was assessed by spectrophotometer with approximately 200 μg of RNA isolated for each sample.

20 μg of total RNA were used to synthesize first-strand cDNA using SuperScript III supermix system for RT-PCR (Invitrogen, catalog #18080-400) according to following protocol: 20 μg of RNA (˜8 μl) and 1 μl gene specific reverse primer with tailing recombination sites (100 um, Kappa, IGG1, IGG2a, IGG2b)+1.25 μl annealing buffer (provided by Invitrogen) were combined in a thin-walled PCR tube and incubated at 65° C. for 5 minutes, then transferred to ice for at least 5 minutes. The oligo-bound RNA sample was then added to the following mixture: 12.5 μl of First Strand reaction mix+2.5 μl enzyme mix on ice. Subsequently, the RT reaction was initiated by incubating at room temperature for 10 minutes and shifting to 50° C. for 60 minutes. After the RT elongation reaction to make cDNA, the samples were heated to 85° C. for 5 minutes to inactivate the enzyme mix and placed on ice. cDNA was then used as template for PCR amplification of variable regions and remaining open reading frame of these antibodies. PCR was performed using first-strand cDNA, modified primers from Mouse Ig-Primer Set (Novagen, catalog #69831-3, and leading recombination sites) and KOD Hot Start Master Mix (Novagen, catalog #71842-4). To amplify heavy chain variable regions, PCR samples were assembled as follows: 2.5 μl 10× reaction buffer+2.0 μl dNTPs+2.0 μl MsSO₄+1 μl cDNA+0.3 μl of KOD enzyme+1.25 μl of one the heavy chain forward primers. To amplify light chain variable regions, PCR samples were assembled as follows: 2.5 μl 10× reaction buffer+2.0 μl dNTPs+2.0 μl MsSO₄+1 μl cDNA+0.3 μl of KOD enzyme+1.25 μl of one the light chain forward primers.

For samples with heavy chain cDNA, the following PCR cycles were used (60-80 cycles, steps 2 through 4):

### 1-Denature 95° C. 2 min.

### 2-Denature 95° C. 20 sec.

### 3-Anneal 47° C. 30 sec.

### 4-Extend 68° C. 3.5 min.

6-Cool 4° C. forever.

For samples with light chain cDNA, the following PCR cycles were used (60-80 cycles, steps 2 through 4):

### 1-Denature 95° C. 2 min.

### 2-Denature 95° C. 20 sec.

### 3-Anneal 55° C. 30 sec.

### 4-Extend 68° C. 2 min.

6-Cool 4° C. forever

PCR products were run on 1.2% agarose gel, and bands migrating at the expected size (700 by for light chains and 1500 by for heavy chains) were excised with a disposable circle punch for DNA extraction. DNA was purified using QIAquick Gel Extraction Kit (Qiagen, catalog #28704) according to the following protocol: gel slices were weighed (˜50 mgs). 10 volumes of buffer QG (˜500 μl) to 1 volume of gel were added to each gel slice. Samples were incubated at 50° C. for 10 minutes until gel slices were completely dissolved, mixing every 2-3 minutes. Samples were then applied to QIAquick column attached to a vacuum manifold. To wash, 1000 μl of buffer PE were added to samples for a total of two washes. Columns were then centrifuged for an additional minute at 21,000×G to completely remove residual ethanol. DNA was eluted by adding 30 μl of H₂O to each column and by spinning for 1 minute at 21,000×G. Purified PCR products were sub-cloned into mammalian expression vectors using sequence and ligation independent cloning. Multiple resultant recombinant plasmids for each hybridoma were then sequenced to identify the entire open reading frame sequences for the heavy and light chain for each antibody. The theoretical molecular weight of the recombinant antibody sequences were compared to the predicted molecular weight of the initial hybridoma to confirm the correct sequence was isolated (Table 9, below).

TABLE 9
VH and VL Amino Acid Sequences of Mouse Anti-BSG2 Monoclonal Antibodies.

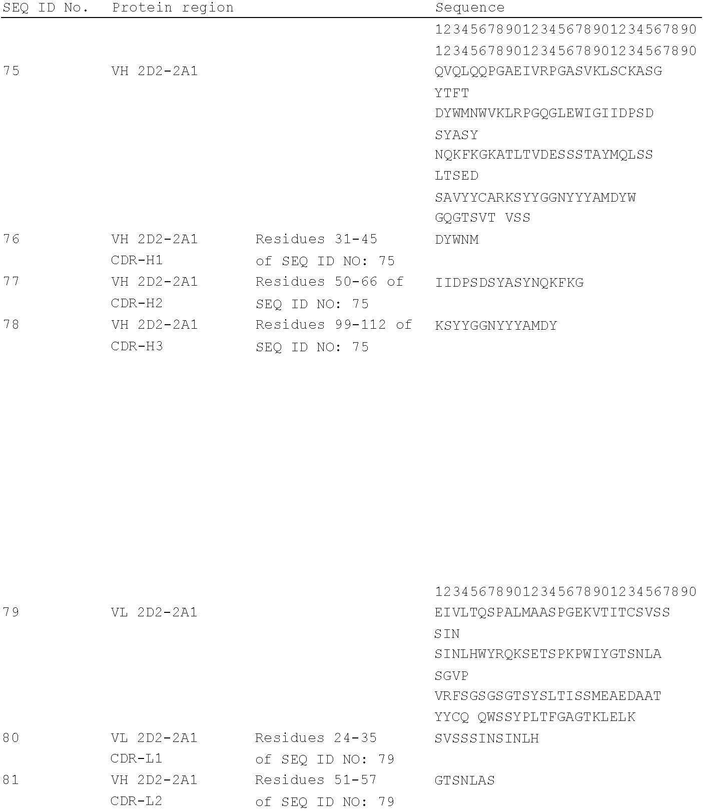

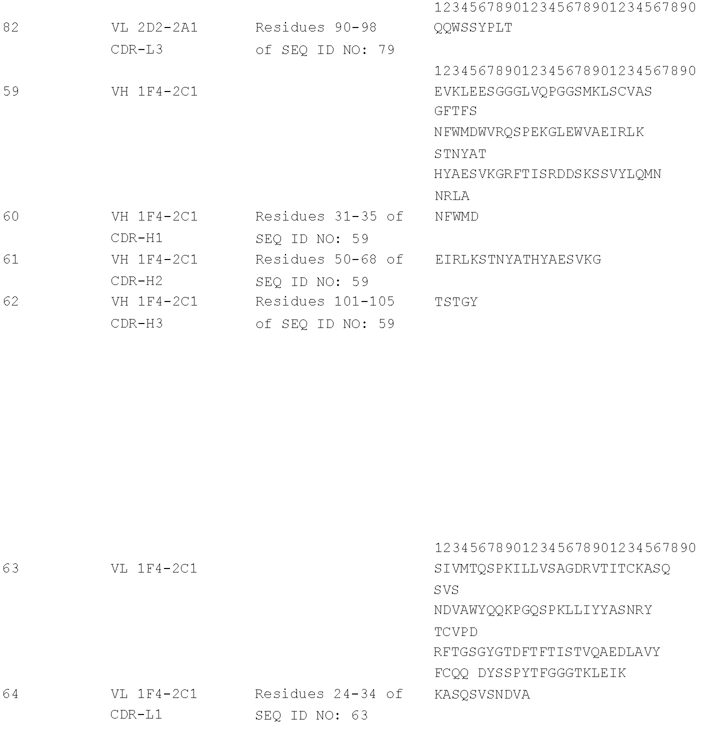

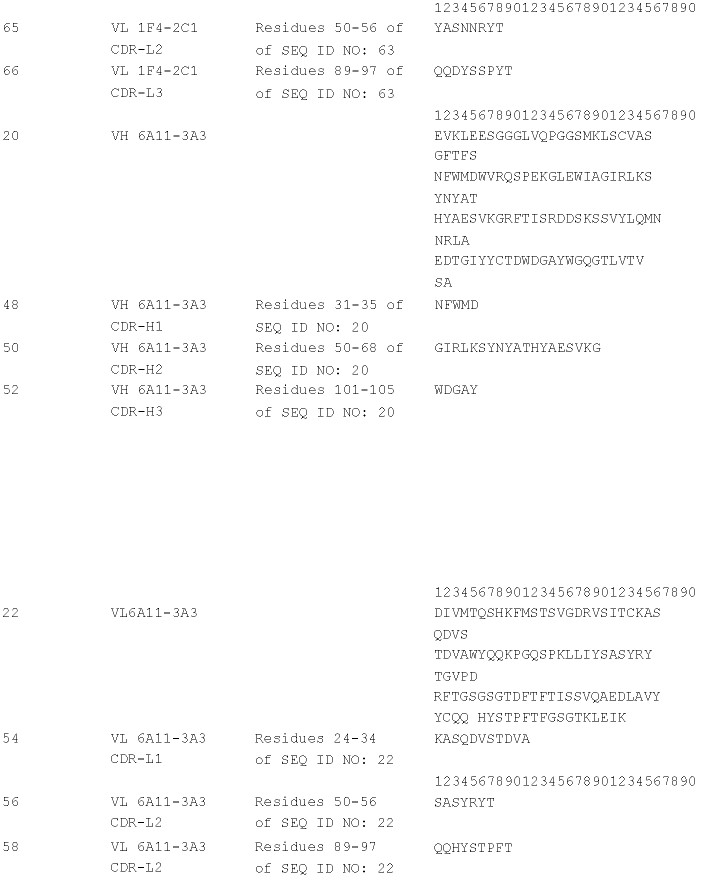

### Example 4

### In vitro Characterization of Mouse Anti-Human BSG2 Monoclonal Antibodies

The BSG2 antibody binding affinities were determined by the BIACORE technology as described in Example 1.2. Table 10 represents the antibody binding kinetics against human and cynomolgus monkey BSG2 extracellular domain (ECD).

TABLE 10
Biacore kinetics on anti-BSG2 monoclonal antibodies.

| | Kinetics on BIACORE | | | | | |
|---|---|---|---|---|---|---|
| | Human BSG2 ECD | | | Cynomolgus BSG2 ECD | | |
| Clone | Kₒₙ | K_{off} | K_{D} | Kₒₙ | K_{off} | K_{D} |
| name | (M⁻¹ s⁻¹) | (s⁻¹) | (M) | (M⁻¹ s⁻¹) | (s⁻¹) | (M) |
| 6A11- | 4.0E+05 | 5.9E−04 | 1.5E−09 | 3.38E+05 | 1.49E−01 | 4.4E−07 |
| 3A3 | | | | | | |
| 1F4- | 4.0E+05 | 1.0E−03 | 2.5E−09 | 3.77E+05 | 6.00E−01 | 1.6E−06 |
| 2C1 | | | | | | |

| | | | | |
|---|---|---|---|---|
| 2D2- | 4.1E+05 | 1.9E−02 | 4.7E−08 | no significant binding |
| 2A1 | | | | |

BSG2 antibody binding activities against whole cells expressing human or cynomolgus monkey BSG2 were assessed by ECL using MSD technology (described in Example 1.3) and the receptor binding assay (described in Example 1.4) and are shown in Table 11.

TABLE 11
Binding affinities of the BSG2 antibodies for cell-surface human
and cynomolgus monkey BSG2.

| | Cell-based MSD binding | | Receptor binding assay | |
|---|---|---|---|---|
| Mouse anti- | (EC₅₀, nM) | | (K_{D}, nM) | |
| human antibody | Human | | Human | |
| Clone name | BSG2 | Cyno BSG2 | BSG2 | Cyno BSG2 |
| 6A11-3A3 | 0.6 | 7.6 | 2.0 | 3.6 |
| 1F4-2C1 | 0.4 | 16.0 | 0.4 | not determined |
| 2D2-2A1 | 0.3 | no binding | 0.15 | no binding |

The ability of mouse anti-human BSG2 antibodies to induce CDC or ADCC using human tumor cell lines was assessed as described in Examples 1.5 and 1.6. As shown in Table 12 below, mouse anti-human BSG2 antibodies promote cell killing of BSG2-expressing human tumor cell lines through CDC and ADCC-based effector function mechanisms. For the CDC assay, up to 87% maximal percent killing was observed in contrast to the negative control IgG1 antibody or a BSG2 antibody expressing mouse IgG1 constant regions. Of note, chimeric mouse BSG2 antibodies (expressing human IgG1 constant regions), which retain binding affinities against BSG2 comparable to its parental mouse antibody, no longer mediate complement lysis but retain effectiveness in directing ADCC by human effector cells at nanomolar EC₅₀ potencies.

TABLE 12
CDC and ADCC activity of BSG2 antibodies.

| | | CDC assay | | ADCC assay |
|---|---|---|---|---|
| | | (% killing at 10 μg/ml Ab) | | (EC₅₀ ng/ml of |
| | | | HepG2 | % specific lysis) |
| | | MiaPaCa-2 | hepatocellular | HepG2 |
| Antibody | | pancreatic | cancer | hepatocellular |
| clone | Isotype | cancer cells | cells | cancer cells |
| Mouse | | | | |
| anti-human | | | | |
| antibody | | | | |
| Neg control | IgG1 | 3 ± 4 | 5 ± 8 | Negative |
| 1F4-2C1 | IgG2a | 87 ± 2 | N/D | N/D |
| 2D2-2A1 | IgG2a | 84 ± 2 | N/D | N/D |
| 6A11-3A3 | IgG2a | 84 ± 2 | 92 ± 0 | Positive |
| 6A11-3A3 | IgG1 | 0 ± 10 | negative | N/D |
| neg control | | | | |
| Chimeric | | | | |
| antibody | | | | |
| 6A11-3A3 | Human | −3 ± 1 | −5 ± 4 | 7.3 ± 2.4 |
| | IgG1 | | | |

“N/D”: not determined.

In addition, crosslinking of cell-surface BSG2 using anti-BSG2 antibodies in combination with an anti-mouse or human IgG antibody resulted in mitochondrial dysfunction, inhibition of Akt phosphorylation, and a decrease in cell viability (described in Examples 1.7 and 1.8). Results from these assays are shown in Tables 13 and 14.

TABLE 13
BSG2 antibody cross-linking decreases the
viability of human tumor cell lines.

| | | MiaPaCa-2 | |
|---|---|---|---|
| | | pancreatic | DOHH-2 |
| | | cancer cells | lymphoma cells |
| Antibody | Isotype | % viability | % viability |
| Mouse | | | |
| anti-human | | | |
| antibody | | | |
| Neg control | IgG2a | 104.8 ± 5.3 | 103.5 ± 7.7 |
| 6A11-3A3 | IgG2a | 55.5 ± 3.0 | 47.0 ± 2.8 |
| | | (Cmax = 125 ng/mL) | (Cmax = 266 ng/mL) |
| Chimeric | | | |
| antibody | | | |
| 6A11-3A3 | Human | 58.2 ± 2.7 | |
| | IgG1 | (Cmax: 500 ng/mL) | |

TABLE 14
Anti-BSG2 antibody cross-linking decreases Akt phosphorylation
and disrupts mitochondrial membrane potential in MiaPaCa
pancreatic human tumor cells.

| | | | Mitochondrial |
|---|---|---|---|
| Mouse | | | membrane |
| anti-human | | | potential |
| antibody | | ph-Akt Ser⁴³⁷ | (DiOC₂(3) stain |
| (500 ng/mL) | Isotype | (signal intensity) | intensity units) |
| Neg control | IgG2a | 20984 ± 178.1 | 2006.3 |
| 6A11-3A3 | IgG2a | 9759.5 ± 986.3 | 810.2 |
| | | (2-fold decrease) | (2.5-fold decrease) |

### Example 5

### BSG2 Antibody Treatment Inhibited Tumor Growth In Vivo

The effect of anti-BSG2 antibodies on tumor growth was evaluated in subcutaneous HepG2, MiaPaCa-2, or DoHH-2 xenograft tumors implanted in SCID female mice (Charles Rivers Labs). Briefly, 2×10⁶ human cancer cells were inoculated subcutaneously into the right hind flank of female SCID mice on study day 0. Administration of antibody (0.5, 30, or 50 mkd, IP, 3× per week for 2 weeks) or Gemcitabine (120 mkd, IP, Q3Dx4) was initiated at the time of size match (days 14-21). The tumors were measured by a pair of calipers twice a week starting on approximately days 14-21 post inoculation and the tumor volumes were calculated according to the formula V=L×W²/2 (V: volume, mm³; L: length, mm. W: width, m) Tumor volume was measured for the duration of the experiment until the mean tumor volume in each group reached an endpoint of >1000 mm³ for HepG2 or >2000 mm³ for MiaPaCa-2 and DoHH-2. Results are shown in Tables 15, 16 and 17. BSG2 antibodies expressing mouse IgG2a constant regions, in contrast to mouse IgG1 constant regions, induced greater anti-tumor effects in hepatocellular, pancreatic, and lymphoma tumor xenograft models.

TABLE 15
Efficacy of anti-BSG2 antibodies in the HepG2 human
hepatocellular cancer xenograft model.

| Mouse anti-human | | | | |
|---|---|---|---|---|
| antibody | Isotype | Dose route, regimen | % TGI^{a} | % ILS^{b} |
| 2D2-2A1 | IgG2a | 0.5mkd IP, 3X/week | 99*** | >626*** |
| | | X2 | | |
| 6A11-3A3 | IgG2a | 0.5mkd IP, 3X/week | 92*** | >367*** |
| | | X2 | | |
| 6A11-3A3 | IgG1 | 0.5mkd IP, 3X/week | 22* | 27* |
| | | X2 | | |
| 1F4-2C1 | IgG2a | 0.5mkd IP, 3X/week | 75*** | >367*** |
| | | X2 | | |
| 1F4-2C1 | IgG1 | 0.5mkd IP, 3X/week | 18 | 13 |
| | | X2 | | |

^{a}Tumor growth inhibition, % TGI = 100 − mean tumor volume of treatment group/mean tumor volume of control group × 100. p values (as indicated by asterisks) are derived from Student's T test comparison of treatment group vs. control group. Based on day 42/45. *p < 0.05, **p < 0.01, ***p < 0.001.
^{b}Increase in life span, % ILS = (T − C)/C × 100, where T = median time to endpoint of treatment group and C = median time to endpoint of control group. p values (as indicated by asterisks) derived from Kaplan Meier log-rank comparison of treatment group vs. treatment control group. Based on an endpoint of 500 mm³. *p < 0.05, **p < 0.01, ***p < 0.001.

TABLE 16
Efficacy of anti-BSG2 antibodies in the MiaPaCa
human pancreatic cancer xenograft model.

| Mouse | | | | |
|---|---|---|---|---|
| anti-human | | | | |
| antibody | Isotype | Dose route, regimen | % TGI^{a} | % ILS^{b} |
| 2D2-2A1 | IgG2a | 50mkd IP, 3X/week X2 | 56*** | 102** |
| 6A11-3A3 | IgG2a | 50mkd IP, 3X/week X2 | 71*** | 176*** |
| 6A11-3A3 + | IgG2a | 50mkd IP, 3X/week X2 + | 92***^{c} | 231***^{c} |
| Gemcitabine | | 120mkd IP, q3 dx4 | | |
| 1F4-2C1 | IgG1 | 50mkd IP, 3X/week X2 | 45** | 41** |
| 1F4-2C1 + | IgG1 | 50mkd IP, 3X/week X2 + | 46***^{d} | 54***^{d} |
| Gemcitabine | | 120mkd IP, q3dx4 | | |

^{a}Tumor growth inhibition, % TGI = 100 − mean tumor volume of treatment group/tumor volume of control group × 100. p values (as indicated by asterisks) are derived from Student's T test comparison of treatment group vs. control group. Based on day 49/57. **p < 0.01, ***p < 0.001.
^{b}Increase in life span, % ILS = (T − C)/C × 100, where T = median time to endpoint of treatment group and C = median time to endpoint of control group. p values (as indicated by asterisks) derived from Kaplan Meier log-rank comparison of treatment group vs. control group. Based on an endpoint of 1000 mm³. **p < 0.01, ***p < 0.001.
^{c}The % TGI of the combination of ML64-6A11-3A3 + gemcitabine was statistically different than MA64-6A11-3A3 alone (p < 0.005) while there was no statistical difference in the % ILS of the combination of ML64-6A11-3A3 + gemcitabine compared to MA64-6A11-3A3 alone.
^{d}Neither the % TGI or % ILS of the combination of EB41-1F4-2C1 + gemcitabine was statistically different than EB41-1F4-2C1 alone.

TABLE 17
Efficacy of anti-BSG2 antibodies in the DoHH-2 human
lymphoma xenograft model.

| Mouse | | | | |
|---|---|---|---|---|
| anti-human | | | | |
| antibody | Isotype | Dose route, regimen | % TGI^{a} | % ILS^{b} |
| 6A11-3A3 | IgG2a | 30mkd IP, 3X/week X2 | 73.4*** | 130.8*** |
| 2D2-2A1 | IgG2a | 30mkd IP, 3X/week X2 | 65.5*** | 84.6*** |
| 1F4-2C1 | IgG1 | 30mkd IP, 3X/week X2 | 52** | 53.8** |

^{a}Tumor growth inhibition, % TGI = 100 − mean tumor volume of treatment group/mean tumor volume of control group × 100. p values (as indicated by asterisks) are derived from Student's T test comparison of treatment group vs. control group. Based on day 27. **p < 0.005, ***p < 0.001
^{b}Increase in life span, % ILS = (T − C)/C × 100, where T = median time to endpoint of treatment group and C = median time to endpoint of control group. p values (as indicated by asterisks) derived from Kaplan Meier log-rank comparison of treatment group vs. control group. Based on an endpoint of 2000 mm³. **p < 0.01, ***p < 0.001

### Example 6

### Humanization of Anti-BSG2 Monoclonal Antibody 3A3

To generate humanized antibody framework back mutations, mutations are introduced into the anti-BSG2 mouse monoclonal antibody 3A3 (also referred to as “ML64-6A11-3A3”) sequences (Table 5) by de novo synthesis of the variable domain and/or using mutagenic primers and PCR, and methods well known in the art (see, e.g., WO 2007/042261, WO 99/54440, Traunecker et al., EMBO J., 10(12):3655-9, 1991, and Lanzavecchia and Scheidegger, Eur. J. Immunol., 17(1):105-11, 1987). Different combinations of back mutations and other mutations are constructed for each variable region. A summary of the proposed design versions of each humanized antibody is set forth below. Residue numbers for all mutations are based on the Kabat numbering system.

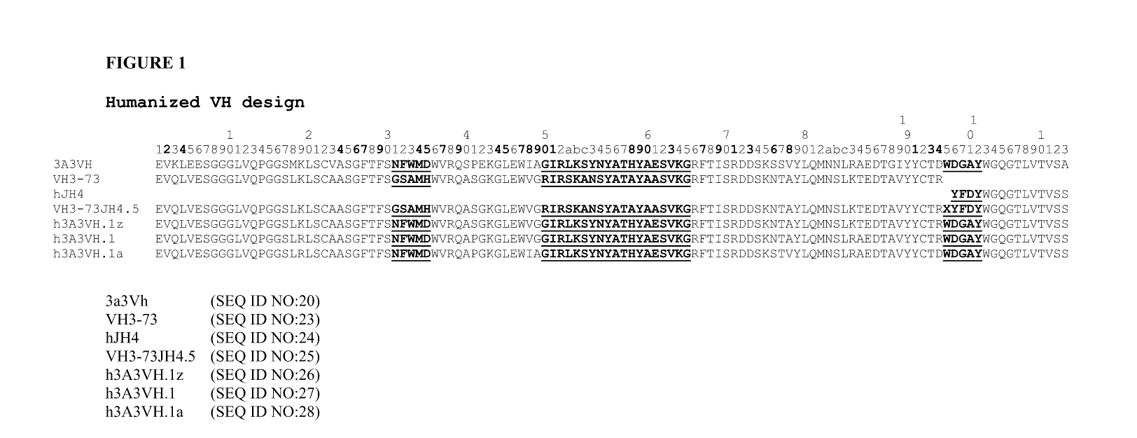

Humanized VH design (as shown in FIG. 1—CDR sequences shown in bold)

VH3-73JH4.5 (SEQ ID NO:25) is a fully human VH with only germline residues from VH3-73 and JH4 separated by a 5 amino acid CDR3.

h3A3VH.1z (SEQ ID NO:26) is a CDR-grafted humanized 3A3 VH containing VH3-73 and hJH4 framework sequences.

h3A3VH.1 (SEQ ID NO:27) is a humanized design incorporating K19R, S41P, K83R, and T84A VH3 framework consensus changes.

h3A3VH.1a (SEQ ID NO:28) is a humanized design containing the consensus changes and all possible framework backmutations below.

| Back-mutation | Effect |
|---|---|
| V48I: | CDR H2 structure |
| G49A: | CDR H2 structure |
| N76S: | CDR H1 structure |
| A78V: | CDR H1 structure |
| R94D: | CDR H3 structure |

### Additional Iterations of Humanized VH Sequence

Sequences having 0, 1, 2, 3, 4, or 5 of the proposed back-mutations in any combination and having 0, 1, 2, 3 or 4 of the suggested VH3 consensus changes can be made to produce additional humanized 3A3 VH sequences with less immunogenicity potential or better overall identity to naturally occurring human VH sequences from the VH3-73 germline sequence.

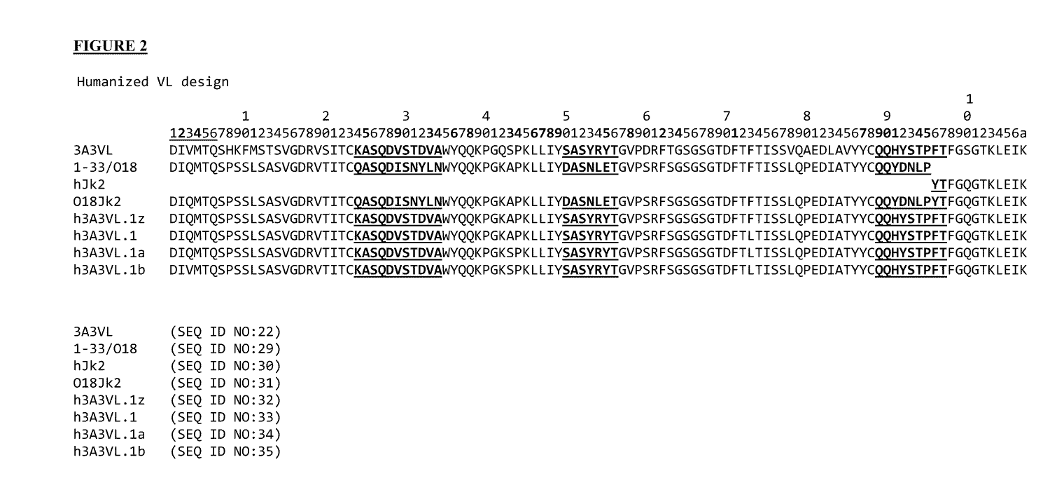

### Humanized VL Design (as Shown in FIG. 2—CDR Sequences Shown in Bold)

O18Jk2 (SEQ ID NO:31) is a fully human VL with only germline residues from O18 and Jk2.

h3A3VL.1z (SEQ ID NO:32) is a direct CDR-grafted humanized 3A3 VL containing O18 and Jk2 framework sequences.

h3A3VL.1 (SEQ ID NO:33) is a humanized design incorporating I83F Vk1 framework consensus change.

h3A3VL.1a (SEQ ID NO:34) is a humanized design containing the consensus change and one possible framework backmutation (A43S).

h3A3VL.1b (SEQ ID NO:35) is a humanized design containing the consensus change and two framework back-mutations, as set forth below.

| Back-mutation | Effect |
|---|---|
| Q3V | Possible CDR L1/L3 structures |
| A43S | VL/VH interface |

### Additional Iterations of Humanized VL Sequence

Sequences having only one of the 2 proposed back-mutations with or without the proposed I83F Vk1 consensus change can be made to achieve better IgG function, less immunogenicity potential, or better overall identity to naturally occurring human VL sequences from the O18 germline sequence. For example, K42Q and/or S60D back mutations can also be made to increase binding capability.

### 6.1

### Construction of CDR Grafted and Humanized Anti Human BSG2 Antibodies

By applying standard methods well known in the art, the CDR sequences of VH and VL chains of monoclonal antibody 6A11-3A3 (see Table 9, above) were grafted into human heavy and light chain acceptor sequences.

Based on sequence VH and VL alignments with the VH and VL sequences of monoclonal antibody 6A11-3A3 of the present invention the following known human sequences are selected:

a) VH3-73 and the joining sequences hJH4 for constructing heavy chain acceptor sequences

b) O18 and hJK2 for constructing light chain acceptor sequences

By grafting the corresponding VH and VL CDRs of 6A11-3A3 into said acceptor sequences, the CDR-grafted, humanized, and modified VH and VL sequences were prepared.

### 6.2

### Construction of Framework Back Mutations in CDR-Grafted Antibodies

To generate humanized antibody framework back mutations, mutations were introduced into the CDR-grafted antibody sequences by de novo synthesis of the variable domain and/or using mutagenic primers and PCR, and methods well known in the art. Different combinations of back mutations and other mutations are constructed for each of the CDR-grafts as follows. Residue numbers for these mutations are based on the Kabat numbering system.

For heavy chains h10F7VH.1z, one or more of the following Vernier and VH/VL interfacing residues were back mutated as follows: V48I, G49A, N76S, A78V, and R94D. Additional mutations include the following: K19R, S41P, K83R, and T84A.

For light chain h10F7Vk.1z and 3z one or more of the following Vernier and VH/VL interfacing residues were back mutated as follows: Q3V and A43S. Additional mutations include the following: F73L and I83F

### 6.3

### Generation of Humanized anti-BSG2 Antibodies Containing Framework Back Mutations in CDR-Grafted Antibodies

The following humanized variable regions of 6A11-3A3 were cloned into IgG expression vectors for functional characterization.

TABLE 18
VH and VL Amino Acid Sequences of Humanized Mouse Anti-BSG2 Monoclonal
Antibody 6A11-3A3

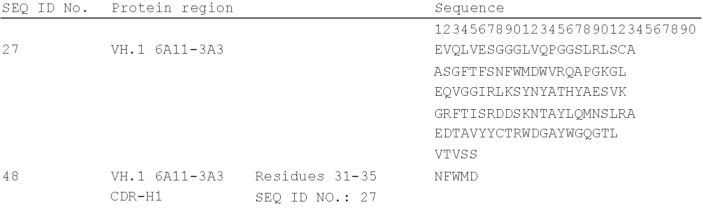

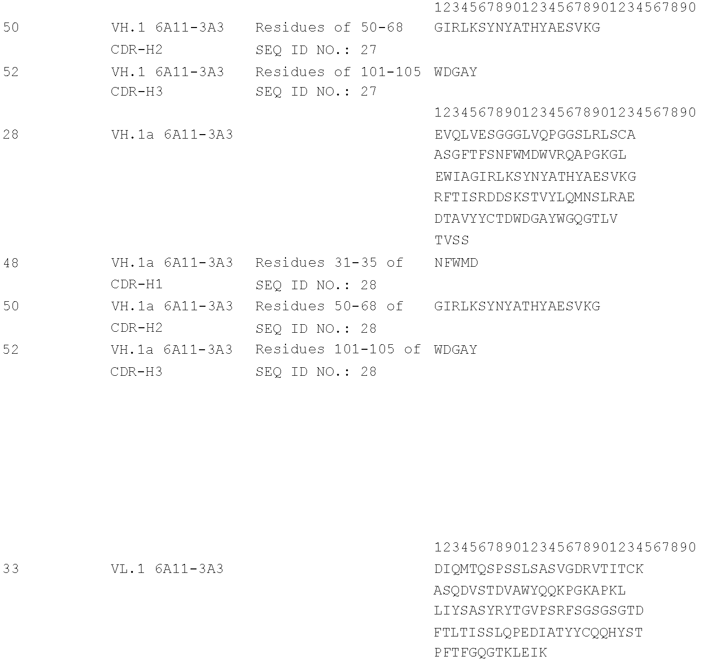

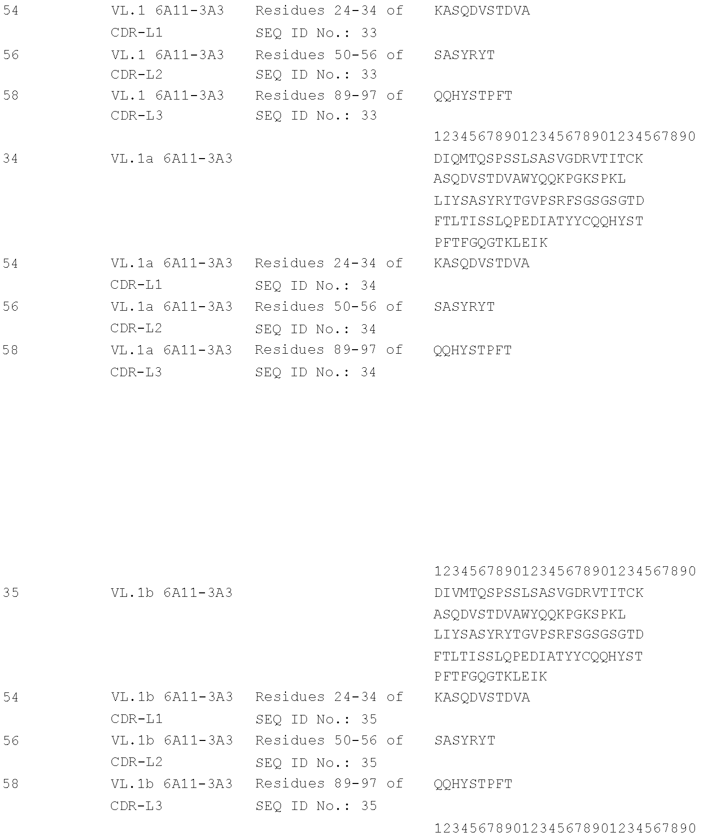

Humanized antibodies from 6A11-3A3 hybridoma clone were generated by combining each heavy chain variant with each light chain variant for a total of 6 variants (Table 19).

TABLE 19
Summary of humanized 6A11-3A3 antibodies generated

| Name | VH/VL combination |
|---|---|
| h3A3-5 | VH.1/VL.1 |
| h3A3-6 | VH.1a/VL.1 |
| h3A3-8 | VH.1/VL.1a |
| h3A3-9 | VH.1a/VL.1a |
| h3A3-11 | VH.1/VL.1b |
| h3A3-12 | VH.1a/VL.1b |

All variants were transiently transfected into 50 mls of HEK 293 6e suspension cell cultures in a ratio of 60% to 40% light to heavy chain construct. 1 mg/ml PEI was used to transfect the cells. Cell supernatants were harvested after six days in shaking flasks, spun down to pellet cells, and filtered through 0.22 μm filters to separate IgG from culture contaminates. Variant binding to human BSG2 was initially assessed through a cell-based binding assay using ECL-MSD as described in Example 1.3.

All variants were batch purified by adding 1 supernatant volume of protein A IgG binding buffer (Thermo Scientific 21001) and 1 ml of rProteinA sepharose fast flow beads (GE Healthcare, 17-1279-04). Supernatants, with beads and buffer added, were rocked overnight at 4° C., and the day after beads were collected by gravity over poly prep chromatography columns (Bio Rad, 731-1550). Once supernatants had passed through the columns the beads were washed with 10 column volumes of binding buffer and IgG was eluted with Immunopure IgG elution buffer (Pierce, 185 1520) and collected in 1 ml aliquots. Fractions containing IgG were pooled and dialyzed in PBS overnight at 4° C.

Purified variants were further characterized for their binding affinities for human BSG2 by ECL-MSD and receptor cell-based binding assays (Examples 1.3 and 1.4), and data is shown in Table 20. Select humanized variants were also tested for their functionality using ADCC and cell viability assays (described in Examples 1.6 and 1.7). These variants showed comparable potencies to the chimeric antibody 6A11-3A3 (Table 21).

TABLE 20
Summary of Binding Activities of Humanized
6A11-3A3 Antibody Variants

| | Cell-based MSD binding | Receptor binding assay |
|---|---|---|
| | (EC₅₀, nM) | (K_{D}, nM) |
| Antibody | Human BSG2 | Human BSG2 |
| 6A11-3A3 parental | 0.6 | 2.0 |
| mouse antibody | | |
| 3A3.5 | weak binding (>100) | N/D |
| 3A3.6 | 2.05 | 5.2 |
| 3A3.8 | 6.90 | N/D |
| 3A3.9 | 1.35 | N/D |
| 3A3.11 | weak binding (>100) | N/D |
| 3A3.12 | 0.96 | 2.3 |

“N/D”: not determined.

TABLE 21
Summary of Functional Activities of Humanized
6A11-3A3 Antibody Variants

| | | | ADCC assay |
|---|---|---|---|
| | | Cross-linking assay | (EC₅₀ ng/ml of |
| | | (% viability, Cmax: | % specific |
| | | 125 ng/ml) MiaPaCa | lysis) HepG2 |
| Antibody | Isotype | pancreatic cells | hepatocellular |
| 6A11-3A3 | IgG1 | 55.5 ± 3.0 | 12.5 ± 1.7 |
| chimeric | | | |
| antibody | | | |
| 3A3.6 | IgG1 | 47.9 ± 2.2 | 16.4 ± 8.5 |
| 3A3.12 | IgG1 | 48.8 ± 3.9 | 9.3 ± 1.0 |
| Neg control | IgG1 | 88.8 ± 4.1 | Negative |

### Example 7

### Humanization of Anti-BSG2 Monoclonal Antibody 2C1

The BSG2 2C1 murine antibody was humanized according to the following procedure.

### 7.1

### Humanization Design for BSG2 2C1 VH Chain

### Identification of Heavy Chain Canonical Structures

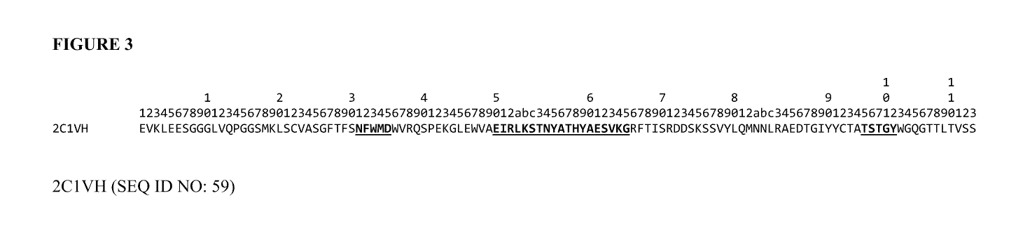

Initially, the canonical structures of the heavy chain CDR's (as set forth in Table 6) were determined in accordance with the procedure set forth in Huang et al. (2005) Methods 36:35-42. For reference, the variable region sequence annotations with Kabat numbering (http://www.bioinf.org.uk/abs/#kabatnum) are set forth in FIG. 3.

The heavy chain canonical structure was determined as follows: 2C1 VH: 1-4

Assign canonical structure:

H1=1 (5 A.A.)

H2=4 (19 A.A. with 52a, 52b, 52c)

Based on the foregoing VH CDR canonical structures, the appropriate acceptor human VH framework sequences were determined to be 3-72, 3-73, and possibly 3-15 and 3-49.

Selection of Human JH Sequence

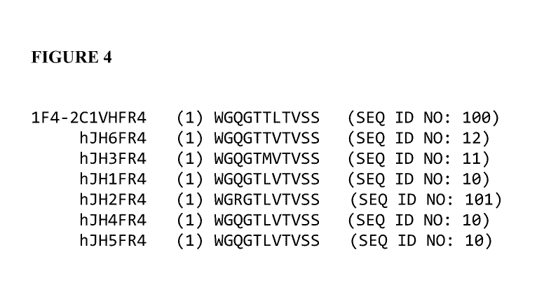

Based on the alignment of possible acceptor human FR4 sequences as compared to the 2C1 FR4 sequence as set forth in FIG. 4, hJH6 has the highest similarity to that of the 2C1 VH sequence. Accordingly, hJH6 was chosen for purposes of the present humanization procedure, although all other hJH FR4 sequences are also possible acceptor sequences.

### Selection of Human VH Germline Sequences for VH Humanization

Initially, residues supporting loop structures and VH/VL interface were identified based on the following tables (as summarized in WO2008021156).

| Residue | | |
|---|---|---|
| # | Score | Reason |
| 2 | 4 | Affects CDR-H1,3* |
| 4 | 3 | Affects CDR-H1,3 |
| 24 | 3 | Affects CDR-H1 |
| 26 | 4 | Affects CDR-H1* |
| 27 | 4 | Affects CDR-H1,3* |
| 29 | 4 | Affects CDR-H1* |
| 34 | 4 | Affects CDR-H1* |
| 35 | 2 | VH/VL interface |
| 37 | 2 | VH/VL interface |
| 39 | 2 | VH/VL interface |
| 44 | 2 | VH/VL interface |
| 45 | 2 | VH/VL interface |
| 47 | 4 | VH/VL interface, CDRL3 |
| 48 | 3 | Affects CDR-H2 |
| 49 | 3 | Affects CDR-H2 |
| 50 | 2 | VH/VL interface |
| 51 | 3 | Affects CDR-H2 |
| 58 | 2 | VH/VL interface |
| 59 | 3 | Affects CDR-H2 |
| 60 | 2 | VH/VL interface |
| 63 | 3 | Affects CDR-H2 |
| 67 | 3 | Affects CDR-H2 |
| 69 | 3 | Affects CDR-H2 |
| 71 | 4 | Affects CDR-H2* |
| 73 | 3 | Affects CDR-H1 |
| 76 | 3 | Affects CDR-H1 |
| 78 | 3 | Affects CDR-H1 |
| 91 | 2 | VH/VL interface |
| 93 | 3 | Affects CDR-H3 |
| 94 | 4 | Affects CDR-H3* |

*Noted as affecting CDR conformation in C. Chothia et al. (1989) “Conformations of Immunoglobulin Hypervariable Regions”, Nature 342: 877-883.

TABLE 2
Residues in the “Vernier” zone (Kabat
numbering)

| Heavy Chain | Light Chain |
|---|---|
| 2 | 2 |
| 27-30 | 4 |
| 47-49 | 35-36 |
| 67 | 46-49 |
| 69 | 64 |
| 71 | 66 |
| 73 | 68-69 |
| 78 | 71 |
| 93-94 | 98 |
| 103 | |

Foote & Winter (1992) JMB 224: 487-499

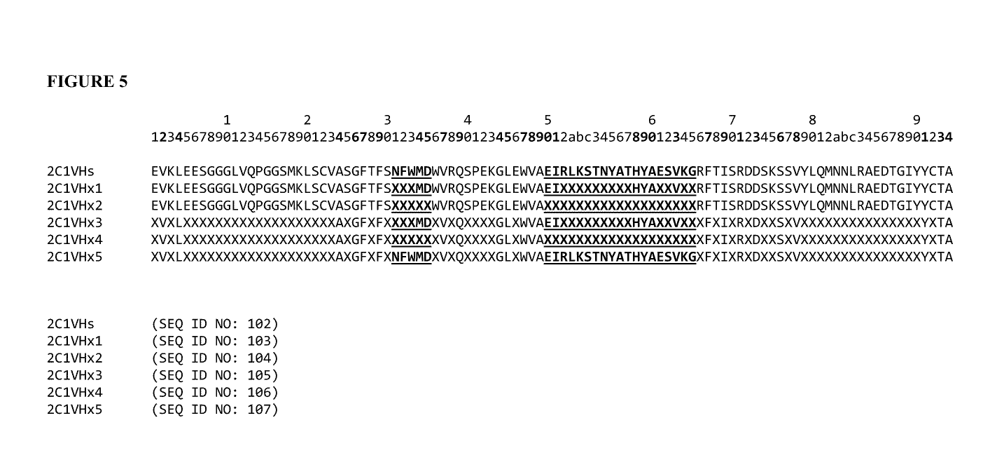

As depicted in FIG. 5, 2C1VHs was aligned against suggested human VH framework sequence acceptors. By referencing the tables above, residues important for loop conformation and VH/VL interface are highlighted in the Kabat numbers by boldface. The CDR sequences are also in bold. Five additional VH sequences (2C1VHx1 to -x5) were created by gradually replacing CDR or framework residues with ‘X’. 2C1VHs is the VH sequence with D and J regions removed.

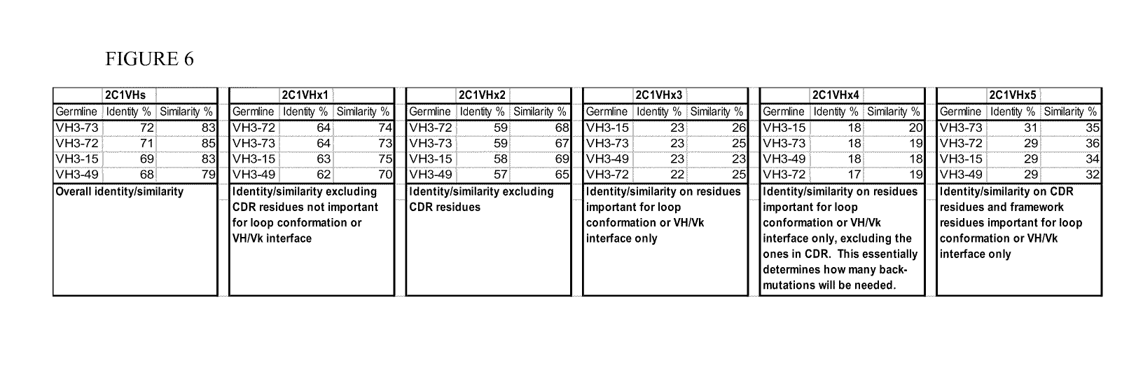

All six sequences were then assigned as ‘profile’ and aligned with human VH sequences in the Align X program of Vector NTI suite. Their identities and similarities to each individual human germline framework sequences are listed in FIG. 6.

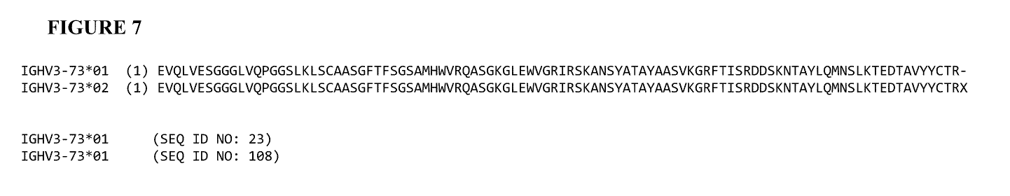

In these alignments, focusing on overall framework or specific residues important for loop conformation and VH/VL interface, the VH3-73 (IGHV3-73) sequence was determined to have the best overall homology to 2C1 VH and, accordingly, was selected as the acceptor human framework for humanization of 2C1 VH. Note that, as reflected in FIG. 7, there is no difference in the protein sequence between IGHV3-73*01 and *02.

Generation of Humanized 2C1 VH Sequences Using VH3-73 as an Acceptor

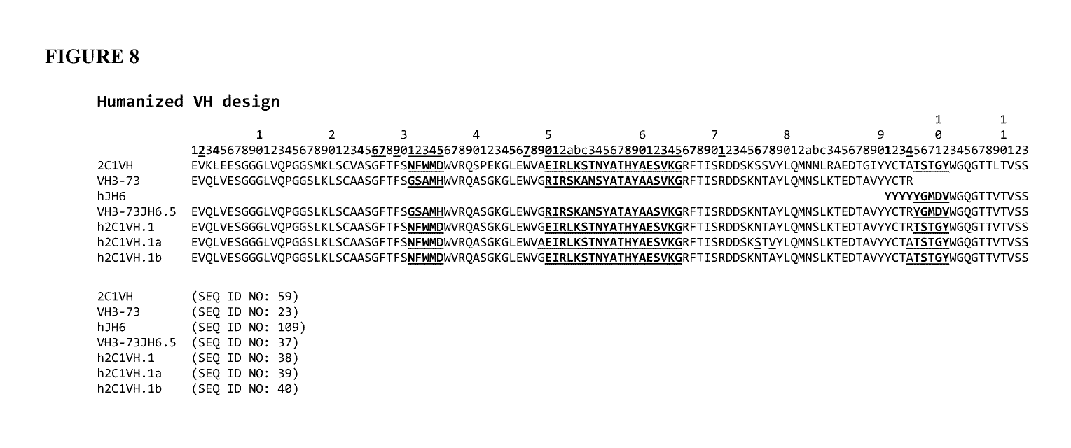

SEQ ID NOs: 38-40 as set forth below and in FIG. 8 represent sequences made in accordance with the above described humanization process and where VH3-73 is used as the acceptor sequence. SEQ ID NO:37, also depicted in FIG. 8, is a fully human VH as set forth below.

VH3-73JH6.5 (SEQ ID NO:37) is a fully human VH with only germline residues from VH3-73 and JH6 separated by a 5 A.A. CDR3.

h2C1VH.1 (SEQ ID NO:38) is a CDR grafted humanized 2C1 VH containing VH3-73 and JH6 framework sequences.

h2C1VH.1a (SEQ ID NO:39) is a humanized design based on 0.1 and contains 4 proposed framework back mutations G49A, N76S, A78V and R94A.

h2C1VH.1b (SEQ ID NO:40) is a compromised design between 0.1 and 0.1a containing one R94A back mutation.

The back mutations and their effects are as summarized below:

| Back-mutation | Effect |
|---|---|
| G49A: | CDR H2 structure |
| N76S: | CDR H1 structure |
| A78V: | CDR H1 structure |
| R94A: | CDR H3 structure |

Sequences having 1, 2, 3 or all 4 of the proposed back-mutations and in any combination can be made to produce additional humanized 2C1 sequences with less immunogenicity potential or better overall identity to naturally occurring human VH sequences from the VH3-73 germline sequence.

Identification of Prevalence of Proposed Back-Mutations in Human Antibodies Originated from VH3-73

Human VH sequences derived from VH3-73 were downloaded from NCBI IgBlast database to generate a sequence logo as follows:

http://www.ncbi.nlm.nih.gov/igblast/retrieveig.html

### Retrieve Ig Sequences

Excluding synthetic Ig molecules=yes
Organism=human
Chain type=VH
Sequence type=protein
Sequence maximal length limit=2000
Sequence minimal length limit=80
Maximal percent identity to germline gene=100
Minimal percent identity to germline gene=80
Functional category=Functional
Limit to germline gene=IGHV3-73
Number of sequences retrieved: 108

These sequences were subsequently downloaded into one batch fasta file, aligned by ClustalW (ftp://ftp.ebi.ac.uk/pub/software/dos/clustalw/ or ftp://ftp-igbmc.u-strasbg.fr/pub/ClustalW/), and visualized by logobar (http://www.biosci.ki.se/groups/tbu/logobar/). The output .eps file was edited by Adobe Illustrator to remove gaps, signal peptide, and constant region sequences.

This analysis was useful to understand whether the proposed four back-mutations and the mouse VH CDR residues are represented in more than 1% of these 108 natural human antibodies that are at least 80% identical to the VH3-73 germline sequence.

Of the 4 proposed back-mutations, each of N76S and A78V were observed in more than 1% of these sequences. The other back-mutations were not present in human antibody sequences from this same germline sequence and should be avoided if possible.

### Evaluation of Potential Immunogenicity of Humanized 2C1 VH Using VH3-73 as an Acceptor by EpiVax EpiMatrix Report

Each of SEQ ID NOs:38-40 were subsequently analyzed in order to compare their predicted immunogenicity. Particular attention was given to the junction between CDRs and

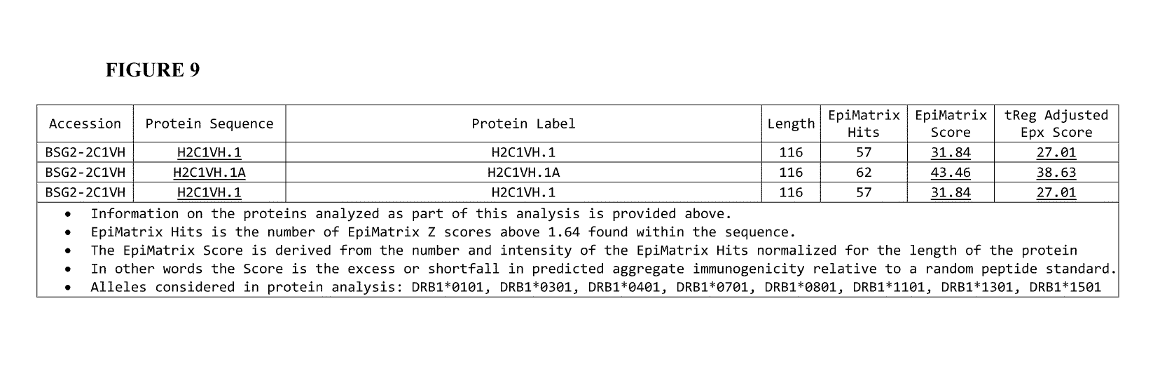

FRs. The analysis was made using the EpiVax database (https://ocs.epivax.com/ispri_abbott/). Results including tReg Adjusted EpiMatrix score are reported in FIG. 9.

Based on the findings depicted in FIG. 9, the humanized 2C1 VH using VH3-73 as an acceptor sequence did not appear to be immunogenic and its immunogenicity was predicted to fall between albumin (Epx Score about −20) and IgG FC region (Epx Score about −40). Non-immunogenic antibodies in this assessment tool have Epx Scores below −50.

### Cluster Analysis

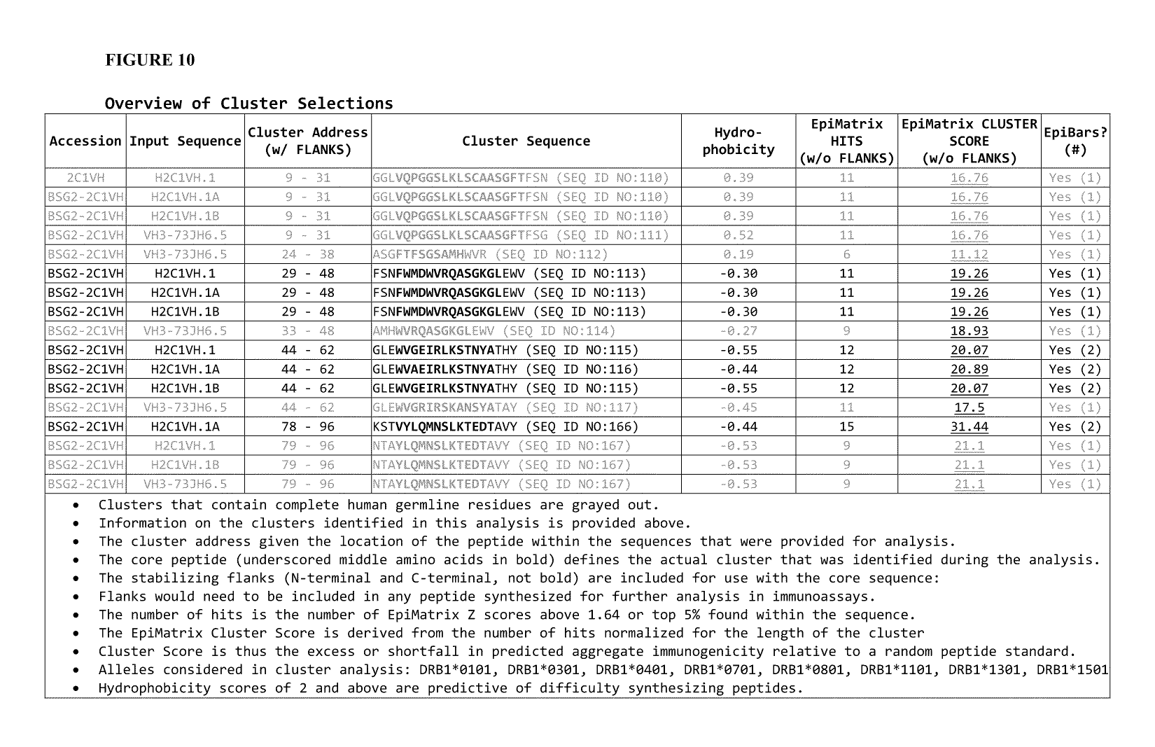

FIG. 10 depicts a cluster selection analysis. Based on the results set forth therein, the FR3 region and the CDR1 to FR2 and FR2 to CDR2 regions were determined to be potential T cell epitopes in the humanized 2C1 VH sequences using VH3-73 as an acceptor sequence. In addition, the FR3.1a sequence was determined to have higher predicted immunogenicity due to back-mutation.

### Conclusions Regarding Humanized 2C1 VH Sequences

In conclusion, the following humanized VH chains were constructed for further analysis:

h2C1VH.1 (SEQ ID NO:38) is a CDR grafted humanized 2C1 VH containing VH3-73 and JH6 framework sequences.

h2C1VH.1a (SEQ ID NO:39) is a humanized design based on 0.1 and contains 4 proposed framework back mutations G49A, N76S, A78V and R94A.

h2C1VH.1b (SEQ ID NO:40) is a compromised design between 0.1 and 0.1a containing one R94A back mutation.

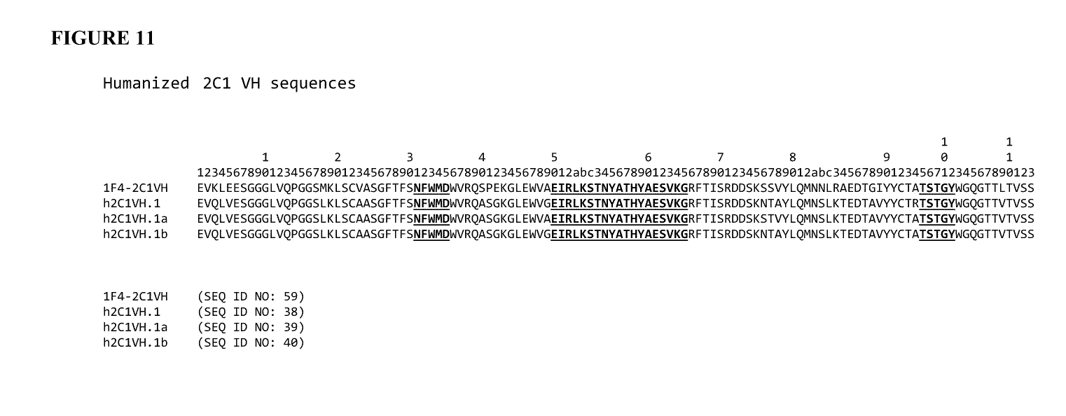

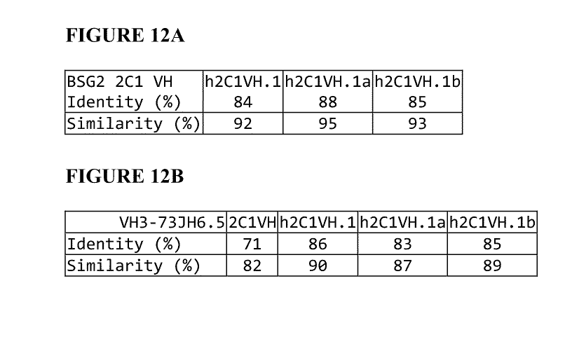

Alignments of these VH sequences are set forth in FIG. 11. Identities and similarities of each of the generated sequences as compared to the 2C1 VH sequence is set forth in FIG. 12A. Identities and similarities of the humanized 2C1 VH sequences using VH3-73 acceptor sequences as compared to VH3-73JH6 are set forth in FIG. 12B.

No N-linked glycosylation pattern (N-{P}-S/T) was found in the proposed constructs.

### 7.2 Humanization Design for BSG2 VL Chain

### Identification of Light Chain Canonical Structures

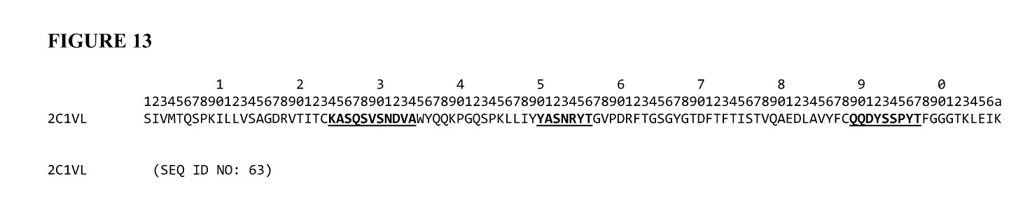

Initially, the canonical structures of the light chain CDRs (as set forth in Table 6) were determined in accordance with the procedure set forth in Huang et al. (2005) Methods 36:35-42. For reference, the variable region sequence annotations with Kabat numbering (http://www.bioinf.org.uk/abs/#kabatnum) are set forth in FIG. 13.

The light chain canonical structure was determined as follows:

2C1 VL: 2-1-1

Assign canonical structure (same above reference):

L1=2 (11 A.A.)

L2=1 (7 A.A.)

L3=1 (9 A.A.; 90Q/N/H, 95P)

Based on the VL CDR canonical structure, the appropriate acceptor human VL framework sequences include those from Vk1, some Vk3, Vk5 and Vk6 subgroups.

### Selection of Human Jk Sequence

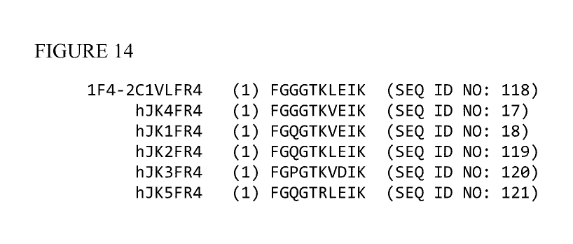

Based on the alignment of possible acceptor human light chain FR4 sequences as compared to the 2C1 light chain FR4 sequence as set forth in FIG. 14, hJK4 was selected for 2C1 VL humanization. All other hJk FR4 sequences were determined to be possible acceptor sequences.

### Selection of Human Vk Germline Sequences for VL Humanization

Initially, residues supporting loop structures and VH/VL interface were identified based on the following tables (as summarized in WO2008021156).

| Residue # | Score | Reason |
|---|---|---|
| 2 | 4 | Affects CDR-L1,3* |
| 4 | 3 | Affects CDR-L1,3 |
| 25 | 4 | Affects CDR-L1* |
| 29 | 4 | Affects CDR-L1,3* |
| 33 | 4 | Affects CDR-L1,3* |
| 34 | 2 | VL/VH interface |
| 36 | 2 | VL/VH interface |
| 38 | 2 | VL/VH interface |
| 43 | 2 | VL/VH interface |
| 44 | 2 | VL/VH interface |
| 46 | 4 | VL/VH interface, CDR-H3 |
| 47 | 3 | Affects CDR-L2 |
| 48 | 4 | Affects CDR-L2* |
| 49 | 2 | VL/VH interface |
| 55 | 2 | VL/VH interface |
| 58 | 3 | Affects CDR-L2 |
| 62 | 3 | Affects CDR-L2 |
| 64 | 4 | Affects CDR-L2* |
| 71 | 4 | Affects CDR-L1* |
| 87 | 2 | VL/VH interface |
| 89 | 2 | VL/VH interface |
| 90 | 4 | Affects CDR-L3* |
| 91 | 2 | VL/VH interface |
| 94 | 2 | VL/VH interface |
| 95 | 4 | Affects CDR-L3* |

*Noted as affecting CDR conformation in C. Chothia et al. (1989) “Conformations of Immunoglobulin Hypervariable Regions,” Nature 342: 877-883.

Residues in the “Vernier” zone (Kabat numbering)

| Heavy Chain | Light Chain |
|---|---|
| 2 | 2 |
| 27-30 | 4 |
| 47-49 | 35-36 |
| 67 | 46-49 |
| 69 | 64 |
| 71 | 66 |
| 73 | 68-69 |
| 78 | 71 |
| 93-94 | 98 |
| 103 | |

Table 2, Foote & Winter (1992) JMB 224: 487-499

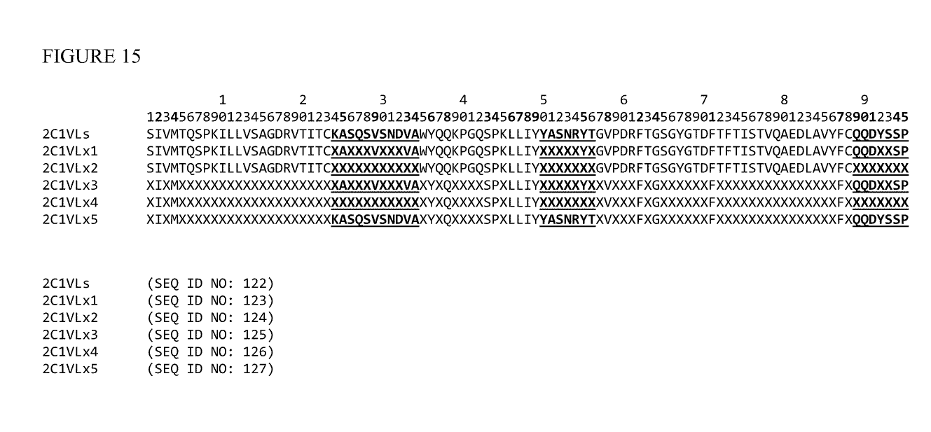

As depicted in FIG. 15, 2C1VLs (the VL sequence with the J region removed) was aligned against suggested human VL framework sequence acceptors. Residues important for loop conformation and VH/VL interface are highlighted in the Kabat numbers by boldface with the CDR sequences in bold.

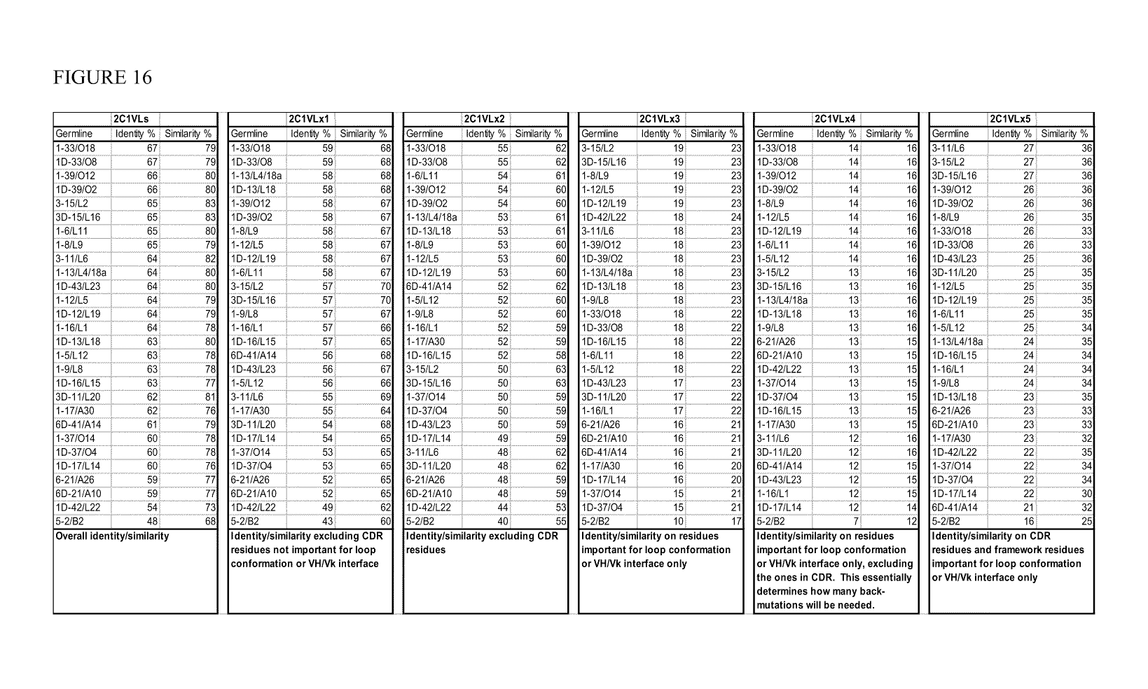

Five additional VL sequences (2C1VLx1 to -x5) were created by gradually replacing CDR or framework residues with “X”. All six sequences were assigned as “profile” and aligned with human Vk sequences in the Align X program of Vector NTI suite. Their identities and similarities to each individual human germline framework sequences are listed in FIG. 16. Only human VL germline sequences having 2-1-1 canonical CDR sequences were considered.

O8/O18 was chosen as the lead human VL germline acceptor sequence from the Vk1 subgroup as a result of its high usage in humans, very good framework identity to 2C1VL and requiring minimal back mutations.

The human VL germline 3-15/L2 (same as 3D15/L16) was selected as the back up acceptor framework for humanization from a different subgroup.

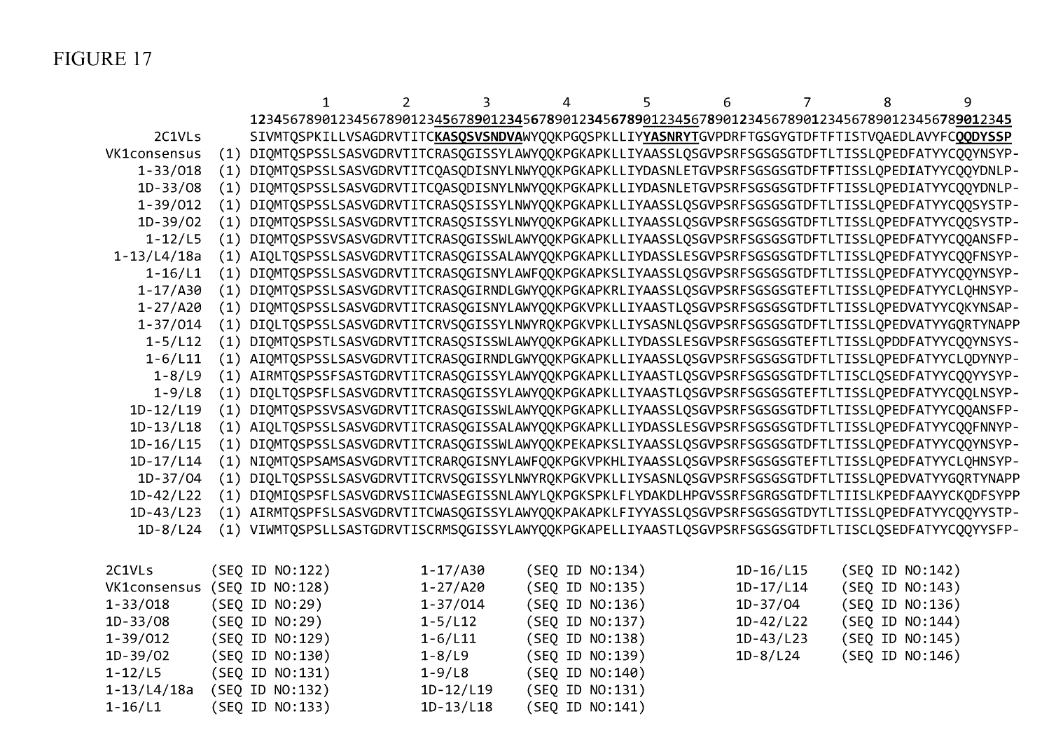

In order to minimize the immunogenicity potential of the humanized sequence, all the human Vk1 germline sequences were aligned to identify potential framework residues in O8/O18 that should be changed into Vk1 consensus, as depicted in FIG. 17. Consensus changes F73L and I83F were identified. However, introducing both changes resulted in grafting 2C1 onto 02/012 framework sequence rather than 08/018. Since O8/O18 is a commonly used germline sequence in humans, neither Vk1 consensus change was introduced.

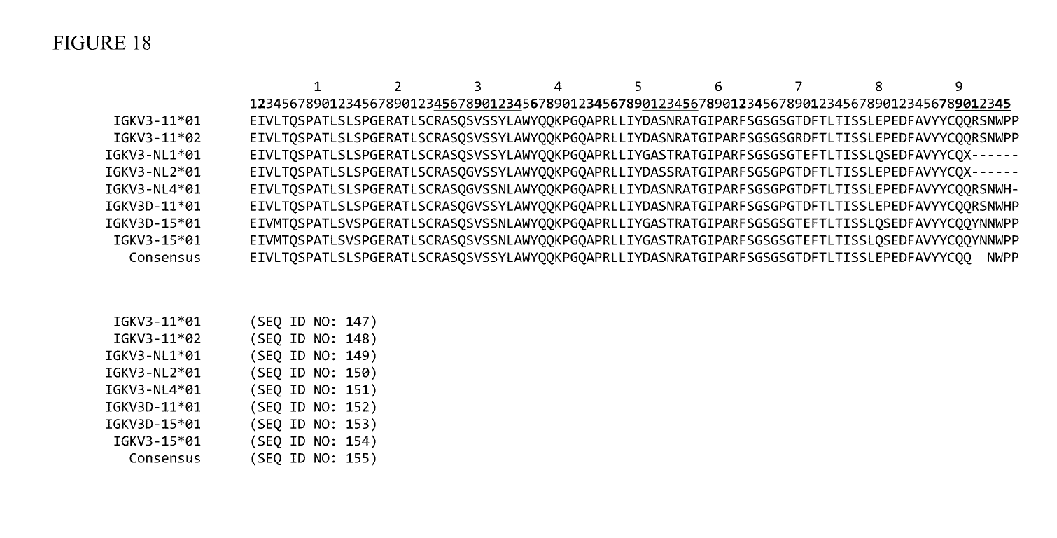

Similarly, all the human Vk3 germline sequences were aligned to identify potential framework residues in IGKV3-15/L2 that should be changed into Vk3 consensus in order to minimize immunogenicity potential, as depicted in FIG. 18. No consensus changes were identified, largely because IGKV3-15 and IGK3-11 are used in roughly equal amounts in humans.

### Generated Humanized 2C1 VL Sequences Using O8/O18 as Acceptor

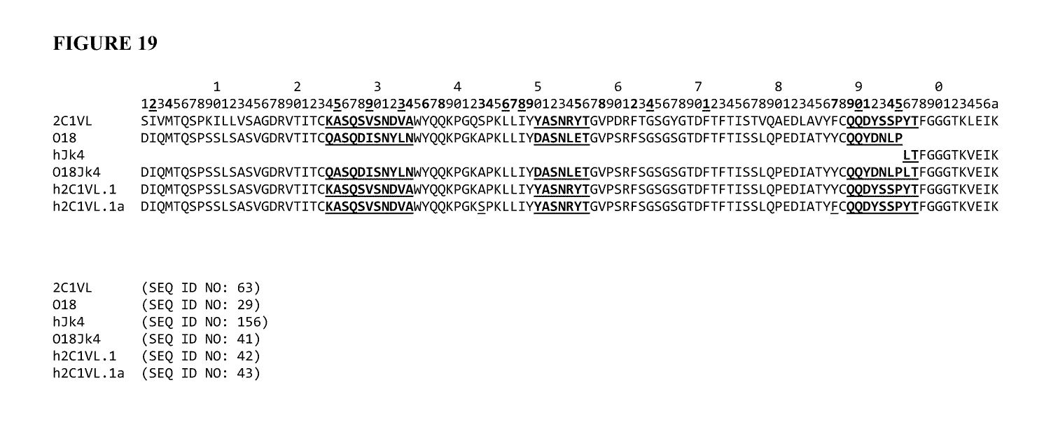

SEQ ID NOs: 42-43 as set forth below and in FIG. 19 represent VL sequences made in accordance with the above-described humanization process where O8/O18 is used as the acceptor sequence. SEQ ID NO:41, also depicted in FIG. 19, is a fully human VL as set forth below.

O18Jk4 (SEQ ID NO:41) is a fully human VL with only germline residues from O18 and Jk4.

h2C1VL.1 (SEQ ID NO:42) is a CDR-grafted humanized 2C1 VL containing O18 and Jk4 framework sequences.

H₂C1VL.1a (SEQ ID NO:43) is a humanized design containing 2 proposed framework back-mutations A43S and Y87F.

The back mutations and their effect are as summarized below:

| Back-mutation | Effect |
|---|---|
| A43S | VL/VH interface |
| Y87F | VL/VH interface |

Sequences having 1 or both of the proposed back-mutations can be made to produce additional humanized 2C1 sequences with less immunogenicity potential or better overall identity to naturally occurring human VL sequences from the O18 germline sequence.

Identification of Prevalence of Proposed Back-Mutations in Human Antibodies Originated from O8/O18

Human Vk sequences derived from O8/O18 were downloaded from NCBI IgBlast database to generate a sequence logo as follows:

http://www.ncbi.nlm.nih.gov/igblast/retrieveig.html

Excluding synthetic Ig molecules=yes

Organism=human

Chain type=VK

Sequence type=protein

Sequence maximal length limit=2000

Sequence minimal length limit=90

Maximal percent identity to germline gene=100

Minimal percent identity to germline gene=80

Functional category=Functional

Limit to germline gene=O18

Number of sequences retrieved: 260

These sequences were subsequently downloaded into one batch fasta file, aligned by

ClustalW (ftp://ftp.ebi.ac.uk/pub/software/dos/clustalw/ or ftp://ftp-igbmc.u-strasbg.fr/pub/ClustalW/), and visualized by logobar (http://www.biosci.ki.se/groups/tbu/logobar/). The output .eps file was edited by Adobe Illustrator to remove gaps, signal peptide, and constant region sequences.

This analysis was useful to understand whether the proposed A43S and Y87F back mutations and the mouse VL CDR residues are represented in more than 1% of these 260 natural human antibodies that are at least 80% identical to the O8/O18 germline sequence. The A43 S was found to be not represented or very rare and should be avoided if possible.

### Evaluation of Potential Immunogenicity of Humanized 2C1 VL Using O8/O18 Acceptor Sequences by EpiVax EpiMatrix Report

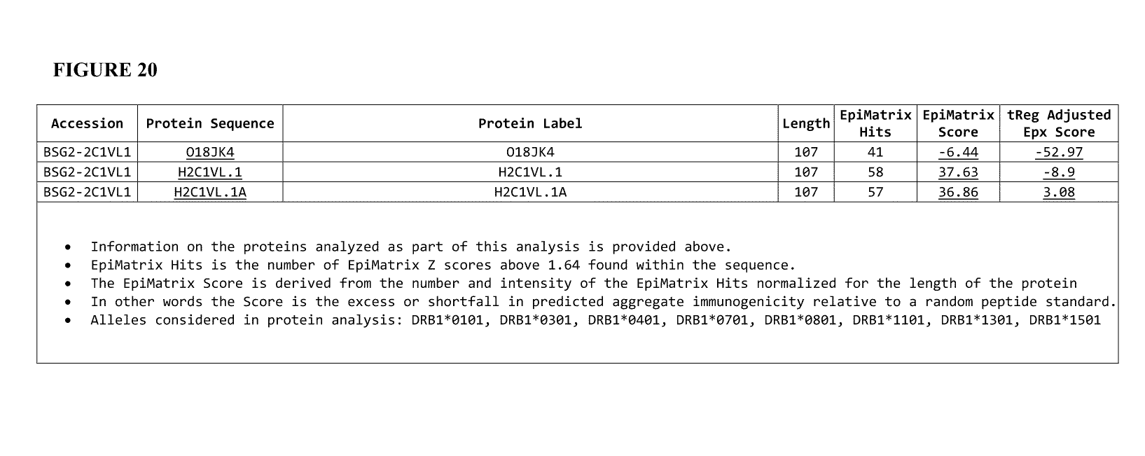

Each of SEQ ID NOs:42-43 were subsequently analyzed in order to compare their predicted immunogenicity. Particular attention was given to the junction between CDRs and FRs. The analysis was made using the EpiVax database (https://ocs.epivax.com/ispri_abbott/). Results including tReg Adjusted EpiMatrix score are reported in FIG. 20.

Based on the findings depicted in FIG. 20, the humanized 2C1 VL using O8/O18 as an acceptor sequence did not appear to be immunogenic and its immunogenicity was predicted to fall between albumin (Epx Score about −20) and IgG FC Region (Epx Score about −40). Non-immunogenic antibodies in this assessment tool have Epx Scores below −50.

### Cluster Analysis

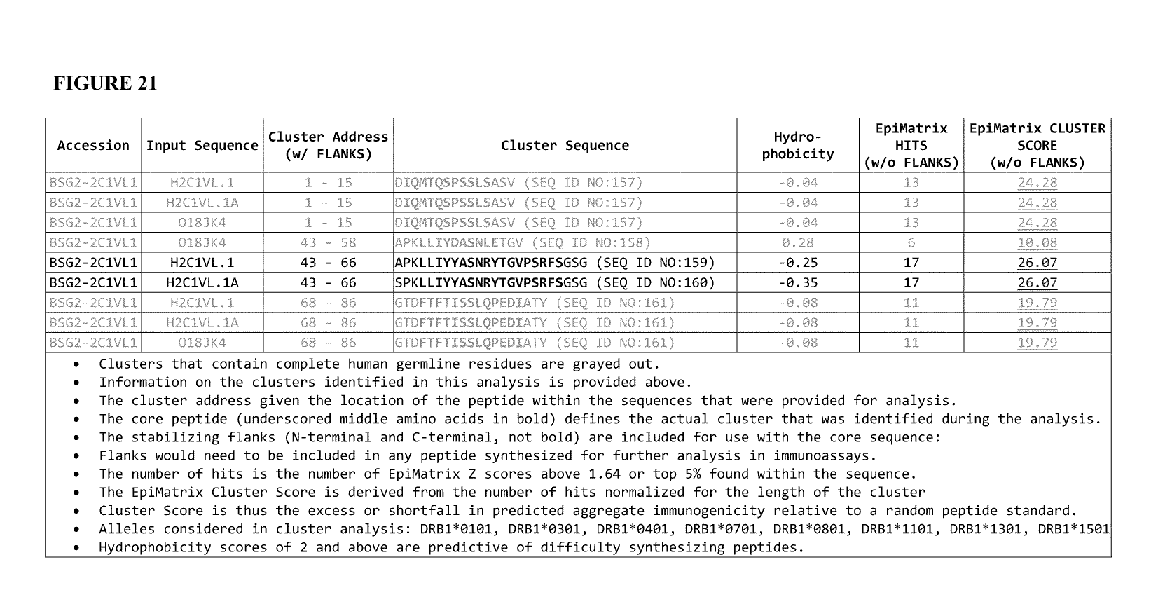

FIG. 21 depicts a cluster selection analysis. Based on the results set forth therein, the FR2 to CDR2 to FR3 region is a potential T cell epitope in the humanized 2C1 VL sequence using O8/O18 as an acceptor sequence.

### Generation of Humanized 2C1 VL Sequences Using 3-15/L2 as Acceptor

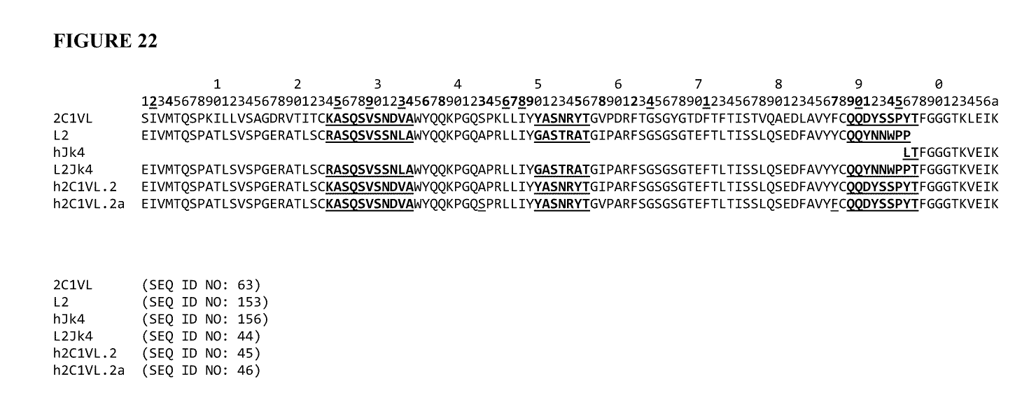

SEQ ID NOs: 45-46 as set forth below and in FIG. 22 represent VL sequences made in accordance with the above-described humanization process where 3-15/L2 is used as the acceptor sequence. SEQ ID NO:44, also depicted in FIG. 22, is a fully human VL as set forth below.

L2Jk4 (SEQ ID NO:44) is a fully human VL with only germline residues from 3-15/L2 and Jk4.

h2C1VL.2 (SEQ ID NO:45) is a direct CDR-grafted humanized 2C1 VL containing 3-15/L2 and Jk4 framework sequences.

H₂C1VL.2a (SEQ ID NO:46) is a humanized design based on 0.2 and contains 3 framework back-mutations (A43S, I58V, and Y87F).

The back mutations and their effects are as summarized below:

| Back-mutation | Effect |
|---|---|
| A43S | VL/VH interface |
| I58V | CDR L2 structure |
| Y87F | VL/VH interface |

Additional sequences having 1, 2 or all 3 of the three proposed back mutations in any combinations can be made in order to test for better IgG function, less immunogenicity potential, or better overall identity to naturally occurring human VL sequences from the 3-15/L2 germline sequence. For example, S60D or A60D back mutations can be made to increase binding capabilities.

Identification of Prevalence of Proposed Back-Mutations in Human Antibodies Originated from IGKV3-15

Human Vk sequences derived from IGKV3-15 were downloaded from NCBI IgBlast database to generate a sequence logo as follows:

http://www.ncbi.nlm.nih.gov/igblast/retrieveig.html

Excluding synthetic Ig molecules=yes

Organism=human

Chain type=VK

Sequence type=protein

Sequence maximal length limit=2000

Sequence minimal length limit=80

Maximal percent identity to germline gene=100

Minimal percent identity to germline gene=90

Functional category=Functional

Limit to germline gene=IGKV3-15

Number of sequences retrieved: 326

These sequences were subsequently downloaded into one batch fasta file, aligned by

ClustalW (ftp://ftp.ebi.ac.uk/pub/software/dos/clustalw/ or ftp://ftp-igbmc.u-strasbg.fr/pub/ClustalW/), and visualized by logobar (http://www.biosci.ki.se/groups/tbu/logobar/). The output .eps file was edited by Adobe Illustrator to remove gaps, signal peptide, and constant region sequences.

This analysis was useful to understand whether the proposed A43S, I58V and Y87F back mutations and the mouse VL CDR residues are represented in more than 1% of these 326 natural human antibodies that are at least 90% identical to the IGKV3-15 germline sequence. All three proposed back mutations were found to be present in human antibodies.

### Evaluation of Potential Immunogenicity of Humanized 2C1 VL Using 3-15/L2 Acceptor Sequences by EpiVax EpiMatrix Report

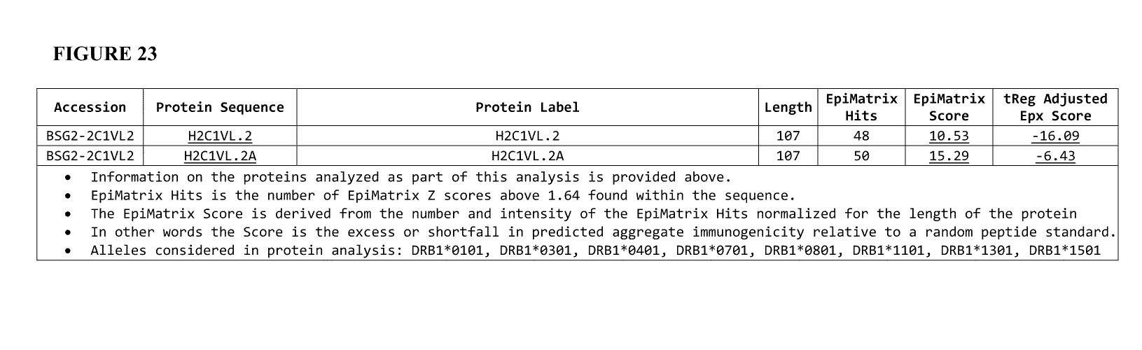

Each of SEQ ID NOs:45-46 were subsequently analyzed in order to compare their predicted immunogenicity. Particular attention was given to the junction between CDRs and FRs. The analysis was made using the EpiVax database (https://ocs.epivax.com/ispri_abbott/). Results including tReg Adjusted EpiMatrix score are reported in FIG. 23.

Based on the findings depicted in FIG. 23, the humanized 2C1 VL using 3-15/L2 as an acceptor sequence did not appear to be immunogenic and its immunogenicity was predicted to be between that of albumin (Epx Score about −20) and IgG FC Region (Epx Score about −40). Non-immunogenic antibodies in this assessment tool have Epx Scores below −50.

### Cluster Analysis

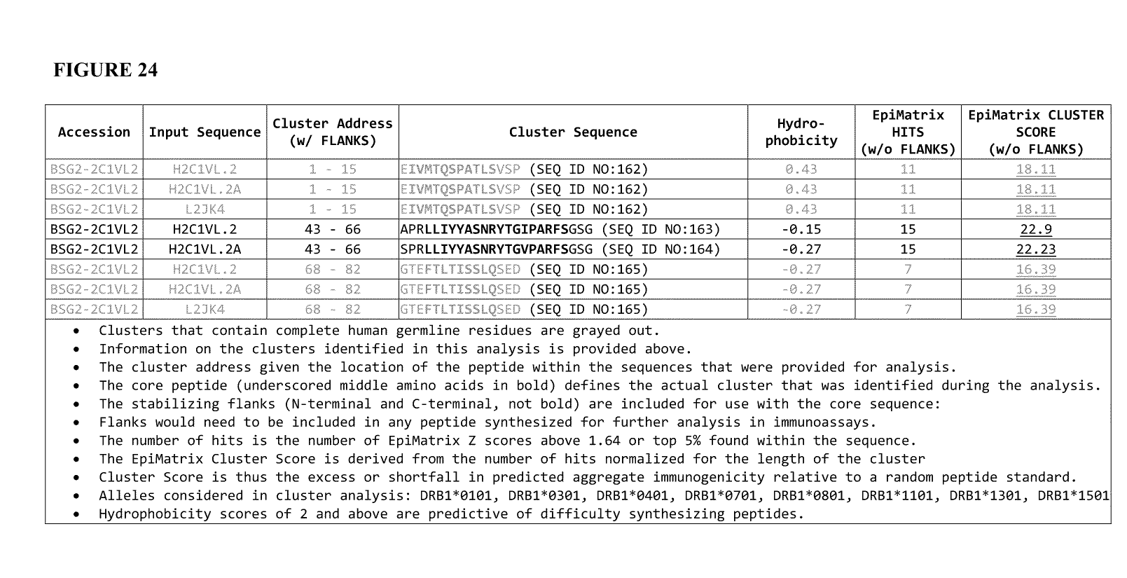

FIG. 24 depicts a cluster selection analysis. Based on the results set forth therein, the FR2 to CDR2 to FR3 region is a potential T cell epitope in the humanized 2C1 VL sequence using 3-15/L2 as an acceptor sequence.

### Humanized BSG2 2C1 VL Chains

In conclusion, the following humanized VL chains were constructed for further analysis:

Version 1 (using O8/O18 as acceptor):

h2C1VL.1 (SEQ ID NO:42) is a CDR-grafted humanized 2C1 VL containing O18 and Jk4 framework sequences.

H₂C1VL.1a (SEQ ID NO:43) is a humanized design containing 2 proposed framework back-mutations A43S and Y87F.

Version 2 (using 3-15/L2 as acceptor):

h2C1VL.2 (SEQ ID NO:45) is a direct CDR-grafted humanized 2C1 VL containing 3-15/L2 and Jk4 framework sequences.

H2C1VL.2a (SEQ ID NO:46) is a humanized design based on 0.2 and contains 3 framework back-mutations (A43S, I58V, and Y87F).

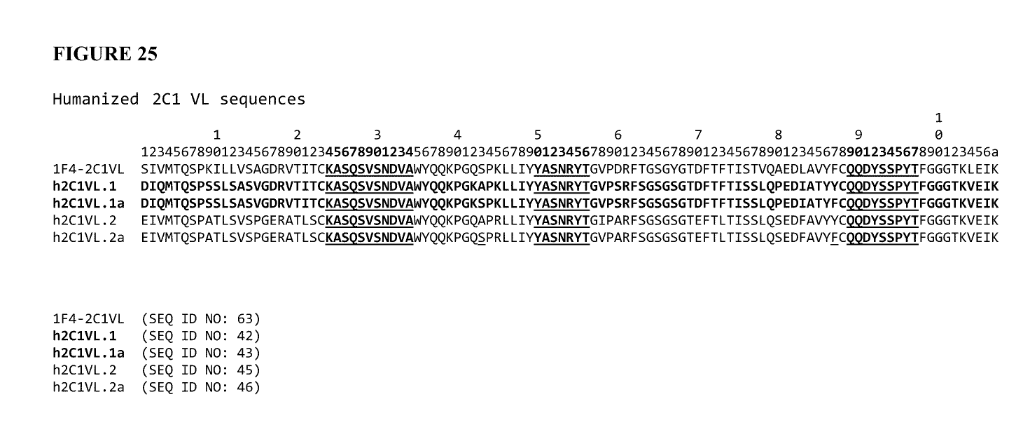

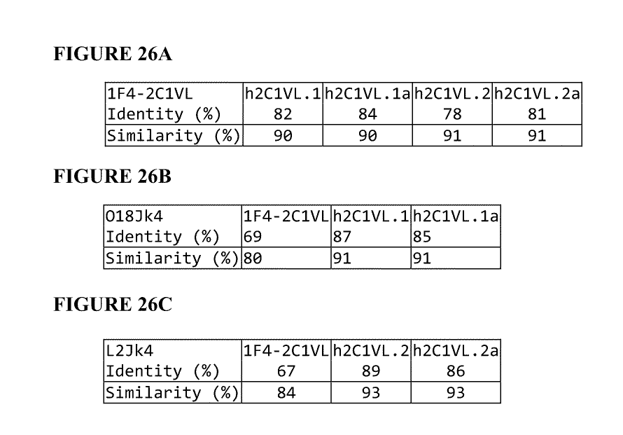

Alignments of these VL sequences are set forth in FIG. 25. Identities and similarities of each of the generated sequences as compared to the 2C1VL are set forth in FIG. 26A. Identities and similarities of the humanized 2C1VL sequences using O8/O18 acceptor sequences as compared to O18Jk4 are set forth in FIG. 26B. Identities and similarities of the humanized 2C1VL sequences using 3-15/L2 acceptor sequences as compared to L2Jk4 are set forth in FIG. 26C.

No N-linked glycosylation pattern (N-(P)-S/T) was found in the proposed VL constructs.

SEQUENCES

| SEQ | | |
|---|---|---|
| ID | | |
| NO | Description | SEQUENCE |
| 1 | hBSG2 | MAAALFVLLGFALLGTHGASGAAGTVFTTV |
| | (isoform 2, short) | EDLGSKILLTCSLNDSATEVTGHRWLKGGV |
| | | VLKEDALPGQKTEFKVDSDDQWGEYSCVFL |
| | | PEPMGTANIQLHGPPRVKAVKSSEHINEGE |
| | | TAMLVCKSESVPPVTDWAWYKITDSEDKAL |
| | | MNGSESRFFVSSSQGRSELHIENLNMEADP |
| | | GQYRCNGTSSKGSDQAIITLRVRSHLAALW |
| | | PFLGIVAEVLVLVTIIFIYEKRRKPEDVLD |
| | | DDDAGSAPLKSSGQHQNDKGKNVRQRNSS |
| | | (UNIPROTKB/SWISS-PROT P35613) |
| 2 | hBSG1 | MAAALFVLLG FALLGTHGAS GAAGFVQAPL |
| | (isoform 1, long) | SQQRWVGGSV ELHCEAVGSP VPEIQWWFEG |
| | | QGPNDTCSQL WDGARLDRVH IHATYHQHAA |
| | | STISIDTLVE EDTGTYECRA SNDPDRNHLT |
| | | RAPRVKWVRA QAVVLVLEPG TVFTTVEDLG |
| | | SKILLTCSLN DSATEVTGHR WLKGGVVLKE |
| | | DALPGQKTEF KVDSDDQWGE YSCVFLPEPM |
| | | GTANIQLHGP PRVKAVKSSE HINEGETAML |
| | | VCKSESVPPV TDWAWYKITD SEDKALMNGS |
| | | ESRFFVSSSQ GRSELHIENL NMEADPGQYR |
| | | CNGTSSKGSD QAIITLRVRS HLAALWPFLG |
| | | IVAEVLVLVT IIFIYEKRRK PEDVLDDDDA GSAPLKSSGQ |
| | | HQNDKGKNVR |
| | | QRNSS |
| | | (UNIPROTKB/SWISS-PROT P35613) |
| 3 | Ig gamma-1 | ASTKGPSVFFLAPSSKSTSGGTAALGCLVKDYFPEPVTVS |
| | constant region | WNSGALTSGVHTFPAVLQSSGLYSLSSVVTVPSSSLGTQT |
| | | YICNVNHKPSNTKVDKKVEPKSCDKTHTCPPCPAPELLG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 4 | Ig gamma-1 | ASTKGPSVFPLAPSSKSTSGGTAALGCLVKDYFPEPVTVS |
| | constant region | WNSGALTSGVHTFPAVLQSSGLYSLSSVVTVPSSSLGTQT |
| | mutant | YICNVNHKPSNTKVDKKVEPKSCDKTHTCPPCPAPEAAG |
| | | GPSVFLFPPKPKDTLMISRTPEVTCVVVDVSHEDPEVKFN |
| | | WYVDGVEVHNAKTKPREEQYNSTYRVVSVLTVLHQDW |
| | | LNGKEYKCKVSNKALPAPIEKTISKAKGQPREPQVYTLPP |
| | | SREEMTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 5 | Ig Kappa constant | RTVAAPSVFIFPPSDEQLKSGTASVVCLLNNFYPREAKVQ |
| | region | WKVDNALQSGNSQESVTEQDSKDSTYSLSSTLTLSKADY |
| | | EKHKVYACEVTHQGLSSPVTKSFNRGEC |
| 6 | Ig Lambda | GQPKAAPSVTLFPPSSEELQANKATLVCLISDFYPGAVTV |
| | constant region | AWKADSSPVKAGVETTTPSKQSNNKYAASSYLSLTPEQW |
| | | KSHRSYSCQVTHEGSTVEKTVAPTECS |
| 7 | VH3-73 FR1 | EVQLVESGGGLVQPGGSLKLSCAASGFTFS |
| 8 | VH3-73 FR2 | WVRQASGKGLEWVG |
| 9 | VH3-73 FR3 | RFTISRDDSKNTAYLQMNSLKTEDTAVYYCTR |
| 10 | JH1/JH4/JH5 FR4 | WGQGTLVTVSS |
| 11 | JH3 FR4 | WGQGTMVTVSS |
| 12 | JH6 FR4 | WGQGTTVTVSS |
| 13 | IGKV1-33/018 | DIQMTQSPSSLSASVGDRVTITC |
| | FR1 | |
| 14 | IGKV1-33/018 | WYQQKPGKAPKLLIY |
| | FR2 | |
| 15 | IGKV1-33/018 | GVPSRFSGSGSGTDFTFTISSLQPEDIATYYC |
| | FR3 | |
| 16 | JK2 FR4 | FGQGTKLEIK |
| 17 | JK4 FR4 | FGGGTKVEIK |
| 18 | JK1 FR4 | FGQGTKVEIK |
| 19 | Ab 3A3 VH- | GAAGTGAAGCTTGAGGAGTCTGGAGGAGGCTTGGTGC |
| | nucleotide | AACCTGGAGGATCCATGAAACTCTCCTGTGTTGCCTCT |
| | sequence | GGATTCACTTTCAGTAACTTCTGGATGGACTGGGTCCG |
| | | CCAGTCTCCAGAGAAGGGGCTTGAGTGGATTGCTGGAA |
| | | TTAGATTGAAATCTTATAATTATGCAACACATTATGCG |
| | | GAGTCTGTGAAAGGGAGGTTCACCATCTCAAGAGATGA |
| | | TTCCAAAAGTAGTGTCTACCTGCAAATGAACAACTTAA |
| | | GAGCTGAAGACACTGGCATTTATTACTGTACCGACTGG |
| | | GACGGGGCTTACTGGGGCCAAGGGACTCTGGTCACTGT |
| | | CTCTGCA |
| 20 | Ab 3A3 VH- | EVKLEESGGGLVQPGGSMKLSCVASGFTFSNFWMDWVR |
| | amino acid | QSPEKGLEWIAGIRLKSYNYATHYAESVKGRFTISRDDSK |
| | sequence | SSVYLQMNNLRAEDTGIYYCTDWDGAYWGQGTLVTVSA |
| 21 | Ab 3A3 VL- | GACATTGTGATGACCCAGTCTCACAAATTCATGTCCAC |
| | nucleotide | ATCAGTAGGAGACAGGGTCAGCATCACCTGCAAGGCC |
| | sequence | AGTCAGGATGTGAGTACTGATGTAGCCTGGTATCAACA |
| | | GAAACCAGGACAATCTCCTAAACTACTGATTTACTCGG |
| | | CATCCTACCGGTACACTGGAGTCCCTGATCGCTTCACT |
| | | GGCAGTGGATCTGGGACGGATTTCACTTTCACCATCAG |
| | | CAGTGTGCAGGCTGAAGACCTGGCAGTTTATTACTGTC |
| | | AGCAACATTATAGTACTCCATTCACGTTCGGCTCGGGG |
| | | ACAAAATTGGAAATAAAA |
| 22 | Ab 3A3 VL- | DIVMTQSHKFMSTSVGDRVSITCKASQDVSTDVAWYQQK |
| | amino acid | PGQSPKLLIYSASYRYTGVPDRFTGSGSGTDFTFTISSVQA |
| | sequence | EDLAVYYCQQHYSTPFTFGSGTKLEIK |
| 23 | VH3-73 | FIG. 1 |
| 24 | hJH4 | FIG. 1 |
| 25 | VH3-73JH4.5 | FIG. 1 |
| 26 | h3A3VH.1z | FIG. 1 |
| 27 | h3A3VH.1 | FIG. 1 |
| 28 | h3A3VH.1a | FIG. 1 |
| 29 | 1-33/O18 | FIG. 2 |
| 30 | hJk2 | FIG. 2 |
| 31 | O18Jk2 | FIG. 2 |
| 32 | h3A3VL.1z | FIG. 2 |
| 33 | h3A3VL.1 | FIG. 2 |
| 34 | h3A3VL.1 a | FIG. 2 |
| 35 | h3A3VL.1 b | FIG. 2 |
| 36 | BSG isoform 4 | MKQSDASPQERVDSDDQWGEYSCVFLPEPMGTANIQLHG |
| | | PPRVKAVKSSEHINEGETAMLVCKSESVPPVTDWAWYKI |
| | | TDSEDKALMNGSESRFFVSSSQGRSELHIENLNMEADPGQ |
| | | YRCNGTSSKGSDQAIITLRVRSHLAALWPFLGIVAEVLVL |
| | | VTIIFIYEKRRKPEDVLDDDDAGSAPLKSSGQHQNDKGKN |
| | | VRQRNSS |
| 37 | VH3-73JH6.5 | FIG. 8 |
| 38 | H2C1VH.1 | FIG. 8 |
| 39 | H2C1VH.1a | FIG. 8 |
| 40 | H2C1VH.1b | FIG. 8 |
| 41 | O18Jk4 | FIG. 19 |
| 42 | H2C1VL.1 | FIG. 19 |
| 43 | H2C1VL.1a | FIG. 19 |
| 44 | L2Jk4 | FIG. 22 |
| 45 | H2C1VL.2 | FIG. 22 |
| 46 | H2C1VL.2a | FIG. 22 |
| 47 | VH 3A3 HC | AACTTCTGGATGGAC |
| | CDR1 (NT) | |
| 48 | VH 3A3 HC | NFWMD |
| | CDR1 (AA) | |
| 49 | VH 3A3 HC | GGAATTAGATTGAAATCTTATAATTATGCAACACATTA |
| | CDR2 (NT) | TGCGGAGTCTGTGAAAGGG |
| 50 | VH 3A3 HC | GIRLKSYNYATHYAESVKG |
| | CDR2 (AA) | |
| 51 | VH 3A3 HC | TGGGACGGGGCTTAC |
| | CDR3 (NT) | |
| 52 | VH 3A3 HC | WDGAY |
| | CDR3 (AA) | |
| 53 | VL 3A3 CDR Ll | AAGGCCAGTCAGGATGTGAGTACTGATGTAGCC |
| | (NT) | |
| 54 | VL 3A3 CDR Ll | KASQDVSTDVA |
| | (AA) | |
| 55 | VL 3A3 CDR L2 | TCGGCATCCTACCGGTACACT |
| | (NT) | |
| 56 | VL 3A3 CDR L2 | SASYRYT |
| | (AA) | |
| 57 | VL 3A3 CDR L3 | CAGCAACATTATAGTACTCCATTCACG |
| | (NT) | |
| 58 | VL 3A3 CDR | QQHYSTPFT |
| | L3(AA) | |
| 59 | VH 2C1 (AA) | EVKLEESGGGLVQPGGSMKLSCVASGFTFS |
| | | NFWMDWVRQSPEKGLEWVAEIRLKSTNYAT |
| | | HYAESVKGRFTISRDDSKSSVYLQMNNLRA |
| | | EDTGIYYCTATSTGYWGQGTTLTVSS |
| 60 | VH 2C1 CDR-H1 | NFWMD |
| | (AA) | |
| 61 | VH 2C1 CDR- | EIRLKSTNYATHYAESVKG |
| | H2(AA) | |
| 62 | VH 2C1 CDR-H3 | TSTGY |
| | (AA) | |
| 63 | VL 2C1(AA) | SIVMTQSPKILLVSAGDRVTITCKASQSVS |
| | | NDVAWYQQKPGQSPKLLIYYASNRYTGVPD |
| | | RFTGSGYGTDFTFTISTVQAEDLAVYFCQQ |
| | | DYSSPYTFGGGTKLEIK |
| 64 | VL 2C1 CDR-L1 | KASQSVSNDVA |
| | (AA) | |
| 65 | VL 2C1 CDR-L2 | YASNRYT |
| | (AA) | |
| 66 | VL 2C1 CDR-L3 | QQDYSSPYT |
| | (AA) | |
| 67 | VH 2C1 (NT) | GAAGTGAAACTGGAAGAAAGCGGCGGCGGCCTGGTGC |
| | | AGCCGGGCGGCAGCATGAAACTGAGCTGCGTGGCGAG |
| | | CGGCTTTACCTTTAGCAACTTTTGGATGGATTGGGTGCG |
| | | CCAGAGCCCGGAAAAAGGCCTGGAATGGGTGGCGGAA |
| | | ATTCGCCTGAAAAGCACCAACTATGCGACCCATTATGC |
| | | GGAAAGCGTGAAAGGCCGCTTTACCATTAGCCGCGATG |
| | | ATAGCAAAAGCAGCGTGTATCTGCAGATGAACAACCTG |
| | | CGCGCGGAAGATACCGGCATTTATTATTGCACCGCGAC |
| | | CAGCACCGGCTATTGGGGCCAGGGCACCACCCTGACCG |
| | | TGAGCAGC |
| 68 | VH 2C1 CDR-H1 | AACTTTTGGATG |
| | (NT) | |
| 69 | VH 2C1 CDR- | GAAATTCGCCTGAAAAGCACCAACTATGCGACCCATTA |
| | H2(NT) | TGCGGAAAGCGTGAAAGGC |
| 70 | VH 2C1 CDR-H3 | ACCAGCACCGGC |
| | (NT) | |
| 71 | VL 2C1(NT) | AGCATTGTGATGACCCAGAGCCCGAAAATTCTGCTGGT |
| | | GAGCGCGGGCGATCGCGTGACCATTACCTGCAAAGCG |
| | | AGCCAGAGCGTGAGCAACGATGTGGCGTGGTATCAGC |
| | | AGAAACCGGGCCAGAGCCCGAAACTGCTGATTTATTAT |
| | | GCGAGCAACCGCTATACCGGCGTGCCGGATCGCTTTAC |
| | | CGGCAGCGGCTATGGCACCGATTTTACCTTTACCATTA |
| | | GCACCGTGCAGGCGGAAGATCTGGCGGTGTATTTTTGC |
| | | CAGCAGGATTATAGCAGCCCGTATACCTTTGGCGGCGG |
| | | CACCAAACTGGAAATTAAA |
| 72 | VL 2C1 CDR-L1 | AAAGCGAGCCAGAGCGTGAGCAACGATGTGGCG |
| | (NT) | |
| 73 | VL 2C1 CDR-L2 | TATGCGAGCAACCGCTATACC |
| | (NT) | |
| 74 | VL 2C1 CDR-L3 | CAGCAGGATTATAGCAGCCCGTATACC |
| | (NT) | |
| 75 | VH 2A1 (AA) | QVQLQQPGAEIVRPGASVKLSCKASGYTFT |
| | | DYWMNWVKLRPGQGLEWIGIIDPSDSYASY |
| | | NQKFKGKATLTVDESSSTAYMQLSSLTSED |
| | | SAVYYCARKSYYGGNYYYAMDYWGQGTSVT |
| | | VSS |
| 76 | VH 2A1 CDR-H1 | DYWMN |
| | (AA) | |
| 77 | VH 2A1 CDR-H2 | IIDPSDSYASYNQKFKG |
| | (AA) | |
| 78 | VH 2A1 CDR-H3 | KSYYGGNYYYAMDY |
| | (AA) | |
| 79 | VL 2A1 (AA) | EIVLTQSPALMAASPGEKVTITCSVSSSIN |
| | | SINLHWYRQKSETSPKPWIYGTSNLASGVP |
| | | VRFSGSGSGTSYSLTISSMEAEDAATYYCQ |
| | | QWSSYPLTFGAGTKLELK |
| 80 | VL 2A1 CDR-L1 | SVSSSINSINLH |
| | (AA) | |
| 81 | VL 2A1 CDR-L2 | GTSNLAS |
| | (AA) | |
| 82 | VL 2A1 CDR-L3 | QQWSSYPLT |
| | (AA) | |
| 83 | VH 2A1 (NT) | CAGGTCCAACTGCAGCAGCCTGGGGCTGAGATTGTGAG |
| | | GCCTGGGGCTTCAGTGAAGCTGTCCTGCAAGGCTTCTG |
| | | GCTACACCTTCACCGACTATTGGATGAACTGGGTGAAA |
| | | CTGAGGCCTGGACAAGGCCTTGAGTGGATTGGAATAAT |
| | | TGATCCTTCTGATAGTTATGCTAGCTACAATCAAAAGTT |
| | | CAAGGGCAAGGCCACATTGACTGTAGACGAGTCCTCCA |
| | | GCACAGCCTACATGCAGCTCAGCAGCCTGACATCTGAG |
| | | GACTCTGCGGTCTATTACTGTGCAAGAAAATCTTACTA |
| | | TGGTGGTAACTACTACTATGCTATGGACTACTGGGGTC |
| | | AAGGAACCTCAGTCACCGTCTCCTCA |
| 84 | VH 2A1 CDR-H1 | GACTATTGGATGAAC |
| | (NT) | |
| 85 | VH 2A1 CDR-H2 | ATAATTGATCCTTCTGATAGTTATGCTAGCTACAATCAA |
| | (NT) | AAGTTCAAGGGC |
| 86 | VH 2A1 CDR-H3 | AAATCTTACTATGGTGGTAACTACTACTATGCTATGGA |
| | (NT) | CTAC |
| 87 | VL 2A1 (NT) | GAAATTGTGCTCACCCAGTCTCCAGCACTCATGGCTGC |
| | | ATCTCCAGGGGAGAAGGTCACCATCACCTGCAGTGTCA |
| | | GCTCAAGTATAAATTCCATCAACTTGCACTGGTACCGG |
| | | CAGAAGTCAGAAACCTCCCCCAAACCCTGGATTTATGG |
| | | CACATCCAACCTGGCTTCTGGAGTCCCTGTTCGCTTCAG |
| | | TGGCAGTGGATCTGGGACCTCTTATTCTCTCACAATCA |
| | | GCAGCATGGAGGCTGAAGATGCTGCCACTTATTACTGT |
| | | CAACAGTGGAGTAGTTACCCACTCACGTTCGGTGCTGG |
| | | GACCAAGCTGGAGCTGAAA |
| 88 | VL 2A1 CDR-L1 | AGTGTCAGCTCAAGTATAAATTCCATCAACTTGCAC |
| | (NT) | |
| 89 | VL 2A1 CDR-L2 | GGCACATCCAACCTGGCTTCT |
| | (NT) | |
| 90 | VL 2A1 CDR-L3 | CAACAGTGGAGTAGTTACCCACTCACG |
| | (NT) | |
| 91 | 2A1HeavyChain | M K C S W V M F L V A T A T G V N S Q V Q L |
| | (AA) | Q Q P G A E I V R P G A S V K L S C K A S G Y |
| | | T F T D Y W M N W V K L R P G Q G L E W I G |
| | | I I D P S D S Y A S Y N Q K F K G K A T L T V |
| | | D E S S S T A Y M Q L S S L T S E D S A V Y Y |
| | | C A R K S Y Y G G N Y Y Y A M D Y W G Q G |
| | | T S V T V S S A K T T A P S V Y P L A P V C G |
| | | D T T G S S V T L G C L V K G Y F P E P V T L |
| | | T W N S G S L S S G V H T F P A V L Q S D L Y |
| | | T L S S S V T V T S S T W P S Q S I T C N V A |
| | | H P A S S T K V D K K I E P R G P T I K P C P |
| | | P C K C P A P N L L G G P S V F I F P P K I K |
| | | D V L M I S L S P I V T C V V V D V S E D D P |
| | | D V Q I S W F V N N V E V H T A Q T Q T H R |
| | | E D Y N S T L R V V S A L P I Q H Q D W M S |
| | | G K E F K C K V N N K D L P A P I E R T I |
| | | S K P K G S V R A P Q V Y V L P P P E E E M T |
| | | K K Q V T L T C M V T D F M P E D I Y V E W |
| | | T N N G K T E L N Y K N T E P V L D S D G S |
| | | Y F M Y S K L R V E K K N W V E R N S Y S C |
| | | S V V H E G L H N H H T T K S F S R T P G K |
| 92 | 2A1 Heavy Chain | ATGAAATGCAGCTGGGTCATGTTCTTGGTAGCAACAGC |
| | (NT) | TACAGGTGTCAACTCCCAGGTCCAACTGCAGCAGCCTG |
| | | GGGCTGAGATTGTGAGGCCTGGGGCTTCAGTGAAGCTG |
| | | TCCTGCAGGCTTCTGGCTACACCTTCACCGACTATTGGA |
| | | TGAACTGGGTGAAACTGAGGCCTGGACAAGGCCTTGA |
| | | GTGGATTGGAATAATTGATCCTTCTGATAGTTATGCTA |
| | | GCTACAATCAAAAGTTCAAGGGCAAGGCCACATTGACT |
| | | GTAGACGAGTCCTCCAGCACAGCCTACATGCAGCTCAG |
| | | CAGCCTGACATCTGAGGACTCTGCGGTCTATTACTGTG |
| | | CAAGAAAATCTTACTATGGTGGTAACTACTACTATGCT |
| | | ATGGACTACTGGGGTCAAGGAACCTCAGTCACCGTCTC |
| | | CTCAGCCAAAACAACAGCCCCATCGGTCTATCCACTGG |
| | | CCCCTGTGTGTGGAGATACAACTGGCTCCTCGGTGACT |
| | | CTAGGATGCCTGGTCAAGGGTTATTTCCCTGAGCCAGT |
| | | GACCTTGACCTGGAACTCTGGATCCCTGTCCAGTGGTG |
| | | TGCACACCTTCCCAGCTGTCCTGCAGTCTGACCTCTACA |
| | | CCCTCAGCAGCTCAGTGACTGTAACCTCGAGCACCTGG |
| | | CCCAGCCAGTCCATCACCTGCAATGTGGCCCACCCGGC |
| | | AAGCAGCACCAAGGTGGACAAGAAAATTGAGCCCAGA |
| | | GGGCCCACAATCAAGCCCTGTCCTCCATGCAAATGCCC |
| | | AGCACCTAACCTCTTGGGTGGACCATCCGTCTTCATCTT |
| | | CCCTCCAAAGATCAAGGATGTACTCATGATCTCCCTGA |
| | | GCCCCATAGTCACATGTGTGGTGGTGGATGTGAGCGAG |
| | | GATGACCCAGATGTCCAGATCAGCTGGTTTGTGAACAA |
| | | CGTGGAAGTACACACAGCTCAGACACAAACCCATAGA |
| | | GAGGATTACAACAGTACTCTCCGGGTGGTCAGTGCCCT |
| | | CCCCATCCAGCACCAGGACTGGATGAGTGGCAAGGAG |
| | | TTCAAATGCAAGGTCAACAACAAAGACCTCCCAGCGCC |
| | | CATCGAGAGAACCATCTCAAAACCCAAAGGGTCAGTA |
| | | AGAGCTCCACAGGTATATGTCTTGCCTCCACCAGAAGA |
| | | AGAGATGACTAAGAAACAGGTCACTCTGACCTGCATGG |
| | | TCACAGACTTCATGCCTGAAGACATTTACGTGGAGTGG |
| | | ACCAACAACGGGAAAACAGAGCTAAACTACAAGAACA |
| | | CTGAACCAGTCCTGGACTCTGATGGTTCTTACTTCATGT |
| | | ACAGCAAGCTGAGAGTGGAAAAGAAGAACTGGGTGGA |
| | | AAGAAATAGCTACTCCTGTTCAGTGGTCCACGAGGGTC |
| | | TGCACAATCACCACACGACTAAGAGCTTCTCCCGGACT |
| | | CCGGGTAAA |
| 93 | 2A1 Light Chain | E I V L T Q S P A L M A A S P G E K |
| | (AA) | V T I T C S V S S S I N S I N L H W Y R Q K S |
| | | E T S P K P W I Y G T S N L A S G V P V R F S |
| | | G S G S G T S Y S L T I S S M E A E D A A T Y |
| | | Y C Q Q W S S Y P L T F G A G T K L E L K R |
| | | A D A A P T V S I F P P S S E Q L T S G G A S |
| | | V V C F L N N F Y P K D I N V K W K I D G S E |
| | | R Q N G V L N S W T D Q D S K D S T Y S M S |
| | | S T L T L T K D E Y E R H N S Y T C E A T |
| | | H K T S T S P I V K S F N R N E C |
| 94 | 2A1 Light Chain | GAAATTGTGCTCACCCAGTCTCCAGCACTCATGGCTGC |
| | (NT) | ATCTCCAGGGGAGAAGGTCACCATCACCTGCAGTGTCA |
| | | GCTCAAGTATAAATTCCATCAACTTGCACTGGTACCGG |
| | | CAGAAGTCAGAAACCTCCCCCAAACCCTGGATTTATGG |
| | | CACATCCAACCTGGCTTCTGGAGTCCCTGTTCGCTTCAG |
| | | TGGCAGTGGATCTGGGACCTCTTATTCTCTCACAATCA |
| | | GCAGCATGGAGGCTGAAGATGCTGCCACTTATTACTGT |
| | | CAACAGTGGAGTAGTTACCCACTCACGTTCGGTGCTGG |
| | | GACCAAGCTGGAGCTGAAACGGGCTGATGCTGCACCA |
| | | ACTGTATCCATCTTCCCACCATCCAGTGAGCAGTTAAC |
| | | ATCTGGAGGTGCCTCAGTCGTGTGCTTCTTGAACAACTT |
| | | CTACCCCAAAGACATCAATGTCAAGTGGAAGATTGATG |
| | | GCAGTGAACGACAAAATGGCGTCCTGAACAGTTGGACT |
| | | GATCAGGACAGCAAAGACAGCACCTACAGCATGAGCA |
| | | GCACCCTCACGTTGACCAAGGACGAGTATGAACGACAT |
| | | AACAGCTATACCTGTGAGGCCACTCACAAGACATCAAC |
| | | TTCACCCATTGTCAAGAGCTTCAACAGGAATGAGTGT |
| 95 | HC CDR2 | (G/E)-I-R-L-K-S-(Y/T)-N-Y-A-T-H-Y-A-E-S-V-K-G |
| | consensus | |
| | sequence | |
| 96 | HC CDR3 | (W/T)-(D/S)-(G/T)-(A/G)-Y |
| | consensus | |
| | sequence | |
| 97 | LC CDR1 | K-A-S-Q-(D/S)-V-S-(T/N)-D-V-A |
| | consensus | |
| | sequence | |
| 98 | LC CDR2 | (S/Y)-A-S-(Y/N)-R-Y-T |
| | consensus | |
| | sequence | |
| 99 | LC CDR3 | Q-Q-(H/D)-Y-S-(T/S)-P-(F/Y)-T |
| | consensus | |
| | sequence | |

We claim:

1. An isolated monoclonal antibody or antigen binding portion thereof that binds to BSG2 and inhibits a BSG2 mediated activity.

2. An isolated monoclonal antibody or antigen binding portion thereof that binds to BSG2 wherein the antibody or antigen binding portion thereof exhibits one or more of the following properties:
(i) inhibition of spermatogenesis;
(ii) inhibition of expression of monocarboxylate transporter expression;
(iii) inhibition of lymphocyte responsiveness;
(iv) inhibition of embryo implantation;
(v) inhibition of formation of neural network;
(vi) inhibition of tumor progression;
(vii) inhibition of tumor angiogenesis; and
(viii) inhibition of production of matrix metalloproteinase.

3. An isolated monoclonal antibody or antigen binding portion thereof, comprising a heavy chain (HC) immunoglobulin variable domain sequence and a light chain (LC) immunoglobulin variable domain sequence, wherein the antibody or antigen binding portion thereof binds to BSG2 and
(A) the HC immunoglobulin variable domain sequence comprises one or more of the following properties:
i) a HC CDR1 that comprises an amino acid sequence as follows:
NFWMD (SEQ ID NO:48);
ii) a HC CDR2 that comprises an amino acid sequence as follows:
(G/E)-I-R-L-K-S-(Y/T)-N-Y-A-T-H-Y-A-E-S-V-K-G-(SEQ ID NO: 95); or
iii) a HC CDR3 that comprises an amino acid sequence as follows:
(W/T)-(D/S)-(G/T)-(A/G)-Y (SEQ ID NO: 96); and
B) the LC immunoglobulin variable domain sequence comprises one or more of the following properties:
i) a LC CDR1 that comprises an amino acid sequence as follows:
K-A-S-Q-(D/S)-V-S-(T/N)-D-V-A (SEQ ID NO: 97);
ii) a LC CDR2 that comprises an amino acid sequence as follows:
(S/Y)-A-S-(Y/N)-R-Y-T (SEQ ID NO: 98); or
iii) a LC CDR3 that comprises an amino acid sequence as follows:
Q-Q-(H/D)-Y-S-(TS)-P-(F/Y)-T (SEQ ID NO: 99).

4. An isolated monoclonal antibody or antigen binding portion thereof that binds to BSG2 wherein the antibody or antigen binding portion thereof comprises a heavy chain variable region comprising an amino acid sequence at least 80% identical to the heavy chain variable region amino acid sequence set forth in SEQ ID NO: 20, 26-28, 38-40, 59 or 75.

5. An isolated monoclonal antibody or antigen binding portion thereof that binds to BSG2 wherein the antibody or antigen binding portion thereof comprises a light chain variable region comprising an amino acid sequence at least 80% identical to the light chain variable region amino acid sequence set forth in SEQ ID NO:22, 32-35, 42-43, 45-46, 63 or 79.

6. An isolated monoclonal antibody or antigen binding portion thereof that binds to BSG2 wherein the antibody or antigen binding portion thereof comprises a heavy chain variable region comprising an amino acid sequence at least 95% identical to the heavy chain variable region amino acid sequence set forth in SEQ ID NO:20, 26-28, 38-40, 59 or 75.

7. An isolated monoclonal antibody or antigen binding portion thereof that binds to BSG2 wherein the antibody or antigen binding portion thereof comprises a light chain variable region comprising an amino acid sequence at least 95% identical to the light chain variable region amino acid sequence set forth in SEQ ID NO:22, 32-35, 42-43, 45-46, 63 or 79.

8. The antibody or antigen binding portion thereof of claim 6 wherein the antibody or antigen binding portion thereof further comprises a light chain variable region comprising an amino acid sequence at least 95% identical to the light chain variable region amino acid sequence set forth in SEQ ID NO:22, 32-35, 42-43, 45-46, 63 or 79.

9. An isolated antibody or antigen binding portion thereof that binds to the epitope which is same or overlapping with the epitope bound by the antibody of claim 1.

10. The isolated monoclonal antibody, or antigen binding portion thereof, of claim 1 that binds to BSG2 and exhibits one or more of the following properties:
(i) inhibition of spermatogenesis;
(ii) inhibition of expression of monocarboxylate transporter expression;
(iii) inhibition of lymphocyte responsiveness;
(iv) inhibition of embryo implantation;
(v) inhibition of formation of neural network;
(vi) inhibition of tumor progression;
(vii) inhibition of tumor angiogenesis; and
(viii) inhibition of production of matrix metalloproteinase.

11. The isolated monoclonal antibody or antigen binding portion thereof of claim 1, wherein the antibody or antigen-binding portion thereof dissociates from human BSG2 extracellular domain with a k_{off} rate constant of 1×10⁻¹ s⁻¹ or less, as determined by surface plasmon resonance.

12. The isolated monoclonal antibody or antigen binding portion thereof of claim 1, wherein the antibody or antigen-binding portion thereof binds to human BSG2 extracellular domain with a K_{D} of 1×10⁻⁷M or less, as determined by surface plasmon resonance.

13. The isolated monoclonal antibody or antigen binding portion thereof of claim 1, wherein the antibody or antigen-binding portion thereof binds to human BSG2 with an EC₅₀ of less than 0.8 nM, as measured by electrochemeluminescence (ECL).

14. The isolated monoclonal antibody or antigen binding portion thereof of claim 1, wherein the antibody or antigen-binding portion thereof binds to human BSG2 with a K_{D} of 2 nM or less, as determined by a receptor binding assay.

15. The isolated monoclonal antibody or antigen binding portion thereof of claim 1, wherein the antibody or antigen-binding portion thereof induces CDC or ADCC mediated killing of tumor cells.

16. The isolated monoclonal antibody or antigen binding portion thereof of claim 15, wherein the antibody or antigen binding portion thereof induces at least 80% killing of tumor cells, as measured by a complement-dependent cytotoxicity assay.

17. The isolated monoclonal antibody or antigen binding portion thereof of claim 1, wherein the antibody or antigen-binding portion thereof decreases Akt phosphorylation and/or disrupts mitochondrial membrane potential in human cancer cells.

18. The isolated monoclonal antibody or antigen binding portion thereof of claim 1, wherein the antibody or antigen-binding portion thereof inhibits tumor growth by at least 50% in a human hepatocellular, human pancreatic cancer or human lymphoma xenograft model.

19. The antibody or antigen binding portion thereof of claim 1, wherein the antibody, or antigen binding portion thereof, is capable of modulating a biological function of one or more targets selected from the group consisting of a cyclophilin, integrin, VEGF, MMP, Akt, and ErbB2.

20. An isolated monoclonal antibody or antigen binding portion thereof that binds to BSG2 wherein the antibody or antigen binding portion thereof comprises: a heavy chain variable region comprising CDR1, CDR2, and CDR3 sequences; and a light chain variable region comprising CDR1, CDR2, and CDR3 sequences wherein the heavy chain variable region CDR3 sequence comprises an amino acid sequence selected from the group consisting of SEQ ID NO:52, SEQ ID NO:62, SEQ ID NO:78 and conservative amino acid substitutions thereof.

21. The antibody or antigen binding portion thereof of claim 20 wherein the light chain variable region CDR3 sequence comprises an amino acid sequence selected from the group consisting of SEQ ID NO:58, SEQ ID NO:66, SEQ ID NO:82 and conservative sequence modifications thereof.

22. The antibody or antigen binding portion thereof of claim 20 or 21 wherein the heavy chain variable region CDR2 sequence comprises an amino acid sequence selected from the group consisting of SEQ ID NO: 50, SEQ ID NO:61, SEQ ID NO:77 and conservative sequence modifications thereof.

23. The antibody or antigen binding portion thereof of claim 22 wherein the light chain variable region CDR2 sequence comprises an amino acid sequence selected from the group consisting of SEQ ID NO: 56, SEQ ID NO:65, SEQ ID NO:81 and conservative sequence modifications thereof.

24. The antibody or antigen binding portion thereof of claim 23 wherein the heavy chain variable region CDR1 sequence comprises an amino acid sequence selected from the group consisting of SEQ ID NO:48, SEQ ID NO:60, SEQ ID NO:76 and conservative sequence modifications thereof.

25. The antibody or antigen binding portion thereof of claim 24 wherein the light chain variable region CDR1 sequence comprises an amino acid sequence selected from the group consisting of SEQ ID NO: 54, SEQ ID NO:64, SEQ ID NO:80 and conservative sequence modifications thereof.

26. An isolated monoclonal antibody or antigen binding portion thereof that binds to BSG2 and comprises a heavy chain variable region CDR1 comprising SEQ ID NO:48; a heavy chain variable region CDR2 comprising SEQ ID NO:50; a heavy chain variable region CDR3 comprising SEQ ID NO: 52; a light chain variable region CDR1 comprising SEQ ID NO: 54; a light chain variable region CDR2 comprising SEQ ID NO: 56; and a light chain variable region CDR3 comprising SEQ ID NO: 58.

27. An isolated monoclonal antibody or antigen binding portion thereof that binds to BSG2 and comprises a heavy chain variable region CDR1 comprising SEQ ID NO:60; a heavy chain variable region CDR2 comprising SEQ ID NO:61; a heavy chain variable region CDR3 comprising SEQ ID NO: 62; a light chain variable region CDR1 comprising SEQ ID NO: 64; a light chain variable region CDR2 comprising SEQ ID NO: 65; and a light chain variable region CDR3 comprising SEQ ID NO: 66.

28. An isolated monoclonal antibody or antigen binding portion thereof that binds to BSG2 and comprises a heavy chain variable region CDR1 comprising SEQ ID NO:76; a heavy chain variable region CDR2 comprising SEQ ID NO:77; a heavy chain variable region CDR3 comprising SEQ ID NO:78; a light chain variable region CDR1 comprising SEQ ID NO: 80; a light chain variable region CDR2 comprising SEQ ID NO:81; and a light chain variable region CDR3 comprising SEQ ID NO:82.

29. An isolated monoclonal antibody or antigen binding portion thereof that binds to BSG2 and comprises a heavy chain variable region comprising CDR1, CDR2, and CDR3 sequences; and a light chain variable region comprising CDR1, CDR2, and CDR3 sequences wherein the heavy chain variable region CDR3 sequence comprises an amino acid sequence which is at least 90% identical to the amino acid sequence selected from the group consisting of SEQ ID NOs: 52, 62 and 78.

30. The antibody of claim 29 wherein the light chain variable region CDR3 sequence comprises an amino acid sequence which is at least 90% identical to the amino acid sequence selected from the group consisting of SEQ ID NOs:58, 66 and 82.

31. The antibody of claim 30 wherein the heavy chain variable region CDR2 sequence comprises an amino acid sequence which is at least 90% identical to the amino acid sequence selected from the group consisting of SEQ ID NOs:50, 61 and 77.

32. The antibody of claim 31 wherein the light chain variable region CDR2 sequence comprises an amino acid sequence which is at least 90% identical to the amino acid sequence selected from the group consisting of SEQ ID NOs: 56, 65 and 81.

33. The antibody of claim 32 wherein the heavy chain variable region CDR1 sequence comprises an amino acid sequence which is at least 90% identical to the amino acid sequence selected from the group consisting of SEQ ID NOs:48, 60 and 76.

34. The antibody of claim 33 wherein the light chain variable region CDR1 sequence comprises an amino acid sequence which is at least 90% identical to the amino acid sequence selected from the group consisting of SEQ ID NOs: 54, 64 and 80.

35. The antibody or antigen binding portion thereof of claim 34 wherein the antibody or antigen binding portion thereof further comprises a heavy chain variable region from human VH3 germline gene.

36. The antibody or antigen binding portion thereof of claim 35 wherein the heavy chain variable region comprises a VH3-73 human germline acceptor sequence.

37. The antibody or antigen binding portion thereof of claim 36 wherein the heavy chain variable region comprises a VH3-73 human germline acceptor sequence and at least one framework consensus or back mutation change selected from the group consisting of V48I, G49A, N76S, A78V, R94A, R94D, K19R, S41P, K83R, T84A, and combinations thereof.

38. The antibody or antigen binding portion thereof of claim 34 further comprising hJH4 or hJH6 as the acceptor human FR4 sequence.

39. The antibody or antigen binding portion thereof of claim 34 wherein the antibody or antigen binding portion thereof further comprises a light chain variable region from human Vk1 or Vk3 germline gene.

40. The antibody or antigen binding portion thereof of claim 34 wherein the antibody or antigen binding portion thereof comprises an O8/O18 or 3-15/L2 acceptor sequence.

41. The antibody or antigen binding portion thereof of claim 40 wherein the light chain variable region comprises a O8/O18 human germline acceptor sequence and at least one framework or back mutation change selected from the group consisting of A43S, Y87F, Q3V, I83F and combinations thereof.

42. The antibody or antigen binding portion thereof of claim 34 wherein the light chain variable region comprises a 3-15/L2 human germline acceptor sequence and at least one framework change selected from the group consisting of A43S, I58V, Y87F and combinations thereof.

43. The antibody or antigen binding portion thereof of claim 34 further comprising hJk2 or hJk4 as the acceptor human FR4 sequence.

44. The antibody or antigen binding portion thereof of claim 1 wherein the antibody is selected from the group consisting of a human antibody, a humanized antibody, a bispecific antibody and a chimeric antibody.

45. The antibody or antigen binding portion thereof of claim 44 wherein the antibody is a humanized antibody.

46. The antibody or antigen binding portion thereof of claim 1 wherein the antibody or antigen binding portion thereof is selected from the group consisting of a Fab, Fab′2, ScFv, SMIP, affibody, avimer, nanobody, and a domain antibody.

47. The antibody or antigen binding portion thereof of claim 1 wherein the antibody isotype is selected from the group consisting of an IgG1, an IgG2, an IgG3, an IgG4, an IgM, an IgA1, an IgA2, an IgAsec, an IgD, and an IgE antibody.

48. An isolated monoclonal antibody or antigen binding portion thereof that binds to BSG2 and comprises a variable heavy chain sequence selected from the group consisting of SEQ ID NOs:27 and 28, and a variable light chain sequence selected from the group consisting of SEQ ID NOs:33, 34 and 35.

49. The antibody or antigen binding portion thereof of claim 48, wherein the antibody or antigen binding portion thereof is of the IgG1 isotype.

50. The antibody or antigen binding portion thereof of claim 48 or 49, wherein the variable heavy chain sequence comprises SEQ ID NO:28 and the variable light chain sequence comprises SEQ ID NO:35.

51. An isolated monoclonal antibody or antigen binding portion thereof that binds to BSG2 and comprises a variable heavy chain sequence selected from the group consisting of SEQ ID NOs:38, 39 and 40, and a variable light chain sequence selected from the group consisting of SEQ ID NOs:42, 43, 45 and 46.

52. A composition comprising the antibody or antigen binding portion of claim 1 in a pharmaceutically acceptable carrier.

53. A composition comprising two or more antibodies of claim 1 wherein the antibodies bind to different epitopes on BSG2.

54. An isolated nucleic acid molecule encoding a heavy chain variable region of an antibody that binds BSG2 wherein said antibody comprises a heavy chain variable region sequence at least 90% identical to a sequence selected from the group consisting of SEQ ID NOs:20, 26-28, 38-40, 59 and 75.

55. An isolated nucleic acid molecule encoding a light chain variable region of an antibody that binds BSG2 wherein said antibody comprises a light chain variable region sequence at least 90% identical to a sequence selected from the group consisting of SEQ ID NOs:22, 32-35, 42-43, 45-46, 63 and 79.

56. An isolated nucleic acid molecule encoding a heavy chain variable region of an antibody that binds BSG2, comprising a nucleotide sequence that hybridizes under highly stringent conditions to a nucleotide sequence encoding a heavy chain variable region selected from the group consisting of SEQ ID NOs:20, 26-28, 38-40, 59 and 75.

57. An isolated nucleic acid molecule encoding a light chain variable region of an antibody that binds BSG2, comprising a nucleotide sequence that hybridizes under highly stringent conditions to a nucleotide sequence encoding a light chain variable region selected from the group consisting of SEQ ID NOs:22, 32-35, 42-43, 45-46, 63 and 79.

58. An expression vector comprising a nucleic acid molecule of any one of claims 54-57.

59. A host cell comprising a nucleic acid molecule of any one of claims 54-57.

60. A hybridoma which produces an antibody or antigen binding portion thereof of claim 1.

61. A kit comprising one or more isolated monoclonal antibodies or antigen binding portions thereof of claim 1 and optionally comprising instructions for use in treating or diagnosing a disease associated with BSG2 activity.

62. A kit comprising one or more isolated monoclonal antibodies or antigen binding portions thereof of claim 1 and optionally comprising instructions for use in treating or diagnosing a disease associated with abnormal angiogenesis.

63. The kit of claim 61 or 62 wherein the disease is selected from the group consisting of cancer, neovascular disease, ocular disease, atherosclerosis, hemangiomas, chronic inflammation and arthritis.

64. A method of inhibiting abnormal angiogenesis in a subject, comprising administering to the subject an isolated monoclonal antibody or antigen binding portion thereof of claim 1 in an amount sufficient to inhibit BSG2 activity.

65. A method of treating a BSG2 mediated disease in a subject, comprising administering to the subject a therapeutically effective amount of an isolated monoclonal antibody or antigen binding portion thereof of claim 1.

66. The method of claim 65 wherein the BSG2 mediated disease is cancer.

67. The method of claim 66 wherein the cancer is selected from the group consisting of liver cancer, pancreatic cancer, lymphoma, leukemia, melanoma, breast cancer, ovarian cancer, liver cancer, renal carcinoma, gastrointestinal cancer, colon cancer, lung cancer, non small cell lung cancer, clear cell sarcoma, prostate cancer, cancer of the oropharynx, cancer of the hypopharynx, esophageal cancer, stomach cancer, cancer of the urinary tract, cancer of the kidney, cancer of the bladder, cancer of the orothelium, cancer of the cervix, cancer of the uterus, cancer of the endocrine glands, thyroid cancer, adrenal cancer, cancer of the pituitary glands, head and neck cancer, skin cancer, brain tumor, tumor of the nerves, meninges, astrocytomas, gliomas, glioblastomas, retinoblastomas, neuromas, neuroblastomas, Schwannomas, and meningiomas.

68. The method of claim 67, wherein the cancer is liver cancer.

69. The method of claim 67, wherein the cancer is pancreatic cancer.

70. The method of claim 67, wherein the cancer is a lymphoma.

71. The method of claim 67, wherein the cancer is prostate cancer.

72. The method of claim 67, wherein the cancer is non-small cell lung cancer.

73. The method of any one of claim 65 wherein the subject is human.

74. The method of any one of claim 65 wherein the antibody or antigen binding portion thereof is administered intravenously, intramuscularly, or subcutaneously to the subject.

75. The method of any one of claim 65 wherein the antibody or antigen binding portion thereof is administered in combination with a second therapeutic agent.

76. The method of claim 75 wherein the second therapeutic agent is a second antibody or antigen binding portion thereof.

77. The method of claim 75 wherein the second therapeutic agent is an anti-cancer agent.

78. The method of claim 77 wherein the anti-cancer agent is selected from the group consisting of an antibody, a biologic, a small molecule, an antimetabolite, an alkylating agent, a topoisomerase inhibitor, a microtubule-targeting agent, a DNA damaging agent, a kinase inhibitor, a protein synthesis inhibitor, an immunotherapeutic, a hormone or analog thereof, a somatostatin analog, a glucocorticoid, an aromatose inhibitor, an mTOR inhibitor, an angiogenesis inhibitor, an anti-EGFR family member inhibitor, a cMet inhibitor, a VEGF inhibitor, an apoptosis inhibitor, a Bcl-2 family member inhibitor, and a tyrosine kinase inhibitor.

79. A method of diagnosing a cancer associated with BSG2 in a subject, comprising (a) contacting ex vivo or in vivo cells from the subject with an isolated monoclonal antibody or antigen binding portion thereof of claim 1, and (b) measuring the level of binding to BSG2 on the cells wherein abnormally high levels of binding to BSG2 indicate that the subject has a cancer associated with BSG2.


SEQUENCE LISTING (167 sequences)

SEQ ID NO: 1 - protein, 269 aa, Homo sapiens
MAAALFVLLGFALLGTHGASGAAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 2 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 3 - protein, 330 aa, Homo sapiens
ASTKGPSVFFLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 107 aa, Homo sapiens [sequence omitted]

SEQ ID NO: 6 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 30 aa, Homo sapiens
EVQLVESGGGLVQPGGSLKLSCAASGFTFS

SEQ ID NO: 8 - protein, 14 aa, Homo sapiens
WVRQASGKGLEWVG

SEQ ID NO: 9 - protein, 32 aa, Homo sapiens
RFTISRDDSKNTAYLQMNSLKTEDTAVYYCTR

SEQ ID NO: 10 - protein, 11 aa, Homo sapiens
WGQGTLVTVSS

SEQ ID NO: 11 - protein, 11 aa, Homo sapiens
WGQGTMVTVSS

SEQ ID NO: 12 - protein, 11 aa, Homo sapiens
WGQGTTVTVSS

SEQ ID NO: 13 - protein, 23 aa, Homo sapiens
DIQMTQSPSSLSASVGDRVTITC

SEQ ID NO: 14 - protein, 15 aa, Homo sapiens
WYQQKPGKAPKLLIY

SEQ ID NO: 15 - protein, 32 aa, Homo sapiens
GVPSRFSGSGSGTDFTFTISSLQPEDIATYYC

SEQ ID NO: 16 - protein, 10 aa, Homo sapiens
FGQGTKLEIK

SEQ ID NO: 17 - protein, 10 aa, Homo sapiens
FGGGTKVEIK

SEQ ID NO: 18 - protein, 10 aa, Homo sapiens
FGQGTKVEIK

SEQ ID NO: 19 - DNA, 348 bp, Artificial Sequence
  Description of Artificial Sequence Synthetic polynucleotide
gaagtgaagcttgaggagtctggaggaggcttggtgcaacctggaggatccatgaaactctcctgtgttgcctctggattcactttcagtaacttctggatggactgggtccgccagtctccagagaaggggcttgagtggattgctggaattagattgaaatcttataattatgcaacacattatgcggagtctgtgaaagggaggttcaccatctcaagagatgattccaaaagtagtgtctacctgcaaatgaacaacttaagagctgaagacactggcatttattactgtaccgactgggacggggcttactggggccaagggactctggtcactgtctctgca

SEQ ID NO: 20 - protein, 116 aa, Artificial Sequence
  Description of Artificial Sequence Synthetic polypeptide
EVKLEESGGGLVQPGGSMKLSCVASGFTFSNFWMDWVRQSPEKGLEWIAGIRLKSYNYATHYAESVKGRFTISRDDSKSSVYLQMNNLRAEDTGIYYCTDWDGAYWGQGTLVTVSA

SEQ ID NO: 21 - DNA, 321 bp, Artificial Sequence
  Description of Artificial Sequence Synthetic polynucleotide
gacattgtgatgacccagtctcacaaattcatgtccacatcagtaggagacagggtcagcatcacctgcaaggccagtcaggatgtgagtactgatgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactccattcacgttcggctcggggacaaaattggaaataaaa

SEQ ID NO: 22 - protein, 107 aa, Artificial Sequence
  Description of Artificial Sequence Synthetic polypeptide
DIVMTQSHKFMSTSVGDRVSITCKASQDVSTDVAWYQQKPGQSPKLLIYSASYRYTGVPDRFTGSGSGTDFTFTISSVQAEDLAVYYCQQHYSTPFTFGSGTKLEIK

SEQ ID NO: 23 - protein, 100 aa, Artificial Sequence
  Description of Artificial Sequence Synthetic polypeptide
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 24 - protein, 15 aa, Artificial Sequence
  Description of Artificial Sequence Synthetic peptide
YFDYWGQGTLVTVSS

SEQ ID NO: 25 - protein, 116 aa, Homo sapiens
  MOD_RES (101)..(101): Any amino acid
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTRXYFDYWGQGTLVTVSS

SEQ ID NO: 26 - protein, 116 aa, Artificial Sequence
  Description of Artificial Sequence Synthetic polypeptide
EVQLVESGGGLVQPGGSLKLSCAASGFTFSNFWMDWVRQASGKGLEWVGGIRLKSYNYATHYAESVKGRFTISRDDSKNTAYLQMNSLKTEDTAVYYCTRWDGAYWGQGTLVTVSS

SEQ ID NO: 27 - protein, 116 aa, Artificial Sequence
  Description of Artificial Sequence Synthetic polypeptide
EVQLVESGGGLVQPGGSLRLSCAASGFTFSNFWMDWVRQAPGKGLEWVGGIRLKSYNYATHYAESVKGRFTISRDDSKNTAYLQMNSLRAEDTAVYYCTRWDGAYWGQGTLVTVSS

SEQ ID NO: 28 - protein, 116 aa, Artificial Sequence
  Description of Artificial Sequence Synthetic polypeptide
EVQLVESGGGLVQPGGSLRLSCAASGFTFSNFWMDWVRQAPGKGLEWIAGIRLKSYNYATHYAESVKGRFTISRDDSKSTVYLQMNSLRAEDTAVYYCTDWDGAYWGQGTLVTVSS

SEQ ID NO: 29 - protein, 95 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 30 - protein, 12 aa, Artificial Sequence
  Description of Artificial Sequence Synthetic peptide
YTFGQGTKLEIK

SEQ ID NO: 31 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 32 - protein, 107 aa, Artificial Sequence
  Description of Artificial Sequence Synthetic polypeptide
DIQMTQSPSSLSASVGDRVTITCKASQDVSTDVAWYQQKPGKAPKLLIYSASYRYTGVPSRFSGSGSGTDFTFTISSLQPEDIATYYCQQHYSTPFTFGQGTKLEIK

SEQ ID NO: 33 - protein, 107 aa, Artificial Sequence
  Description of Artificial Sequence Synthetic polypeptide
DIQMTQSPSSLSASVGDRVTITCKASQDVSTDVAWYQQKPGKAPKLLIYSASYRYTGVPSRFSGSGSGTDFTLTISSLQPEDIATYYCQQHYSTPFTFGQGTKLEIK

SEQ ID NO: 34 - protein, 107 aa, Artificial Sequence
  Description of Artificial Sequence Synthetic polypeptide
DIQMTQSPSSLSASVGDRVTITCKASQDVSTDVAWYQQKPGKSPKLLIYSASYRYTGVPSRFSGSGSGTDFTLTISSLQPEDIATYYCQQHYSTPFTFGQGTKLEIK

SEQ ID NO: 35 - protein, 107 aa, Artificial Sequence
  Description of Artificial Sequence Synthetic polypeptide
DIVMTQSPSSLSASVGDRVTITCKASQDVSTDVAWYQQKPGKSPKLLIYSASYRYTGVPSRFSGSGSGTDFTLTISSLQPEDIATYYCQQHYSTPFTFGQGTKLEIK

SEQ ID NO: 36 - protein, 205 aa, Homo sapiens
MKQSDASPQERVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 37 - protein, 116 aa, Homo sapiens
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTRYGMDVWGQGTTVTVSS

SEQ ID NO: 38 - protein, 116 aa, Artificial Sequence
  Description of Artificial Sequence Synthetic polypeptide
EVQLVESGGGLVQPGGSLKLSCAASGFTFSNFWMDWVRQASGKGLEWVGEIRLKSTNYATHYAESVKGRFTISRDDSKNTAYLQMNSLKTEDTAVYYCTRTSTGYWGQGTTVTVSS

SEQ ID NO: 39 - protein, 116 aa, Artificial Sequence
  Description of Artificial Sequence Synthetic polypeptide
EVQLVESGGGLVQPGGSLKLSCAASGFTFSNFWMDWVRQASGKGLEWVAEIRLKSTNYATHYAESVKGRFTISRDDSKSTVYLQMNSLKTEDTAVYYCTATSTGYWGQGTTVTVSS

SEQ ID NO: 40 - protein, 116 aa, Artificial Sequence
  Description of Artificial Sequence Synthetic polypeptide
EVQLVESGGGLVQPGGSLKLSCAASGFTFSNFWMDWVRQASGKGLEWVGEIRLKSTNYATHYAESVKGRFTISRDDSKNTAYLQMNSLKTEDTAVYYCTATSTGYWGQGTTVTVSS

SEQ ID NO: 41 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 42 - protein, 107 aa, Artificial Sequence
  Description of Artificial Sequence Synthetic polypeptide
DIQMTQSPSSLSASVGDRVTITCKASQSVSNDVAWYQQKPGKAPKLLIYYASNRYTGVPSRFSGSGSGTDFTFTISSLQPEDIATYYCQQDYSSPYTFGGGTKVEIK

SEQ ID NO: 43 - protein, 107 aa, Artificial Sequence
  Description of Artificial Sequence Synthetic polypeptide
DIQMTQSPSSLSASVGDRVTITCKASQSVSNDVAWYQQKPGKSPKLLIYYASNRYTGVPSRFSGSGSGTDFTFTISSLQPEDIATYFCQQDYSSPYTFGGGTKVEIK

SEQ ID NO: 44 - protein, 107 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPTFGGGTKVEIK

SEQ ID NO: 45 - protein, 107 aa, Artificial Sequence
  Description of Artificial Sequence Synthetic polypeptide
EIVMTQSPATLSVSPGERATLSCKASQSVSNDVAWYQQKPGQAPRLLIYYASNRYTGIPARFSGSGSGTEFTLTISSLQSEDFAVYYCQQDYSSPYTFGGGTKVEIK

SEQ ID NO: 46 - protein, 107 aa, Artificial Sequence
  Description of Artificial Sequence Synthetic polypeptide
EIVMTQSPATLSVSPGERATLSCKASQSVSNDVAWYQQKPGQSPRLLIYYASNRYTGVPARFSGSGSGTEFTLTISSLQSEDFAVYFCQQDYSSPYTFGGGTKVEIK

SEQ ID NO: 47 - DNA, 15 bp, Artificial Sequence
  Description of Artificial Sequence Synthetic oligonucleotide
aacttctggatggac

SEQ ID NO: 48 - protein, 5 aa, Artificial Sequence
  Description of Artificial Sequence Synthetic peptide
NFWMD

SEQ ID NO: 49 - DNA, 57 bp, Artificial Sequence
  Description of Artificial Sequence Synthetic oligonucleotide
ggaattagattgaaatcttataattatgcaacacattatgcggagtctgtgaaaggg

SEQ ID NO: 50 - protein, 19 aa, Artificial Sequence
  Description of Artificial Sequence Synthetic peptide
GIRLKSYNYATHYAESVKG

SEQ ID NO: 51 - DNA, 15 bp, Artificial Sequence
  Description of Artificial Sequence Synthetic oligonucleotide
tgggacggggcttac

SEQ ID NO: 52 - protein, 5 aa, Artificial Sequence
  Description of Artificial Sequence Synthetic peptide
WDGAY

SEQ ID NO: 53 - DNA, 33 bp, Artificial Sequence
  Description of Artificial Sequence Synthetic oligonucleotide
aaggccagtcaggatgtgagtactgatgtagcc

SEQ ID NO: 54 - protein, 11 aa, Artificial Sequence
  Description of Artificial Sequence Synthetic peptide
KASQDVSTDVA

SEQ ID NO: 55 - DNA, 21 bp, Artificial Sequence
  Description of Artificial Sequence Synthetic oligonucleotide
tcggcatcctaccggtacact

SEQ ID NO: 56 - protein, 7 aa, Artificial Sequence
  Description of Artificial Sequence Synthetic peptide
SASYRYT

SEQ ID NO: 57 - DNA, 27 bp, Artificial Sequence
  Description of Artificial Sequence Synthetic oligonucleotide
cagcaacattatagtactccattcacg

SEQ ID NO: 58 - protein, 9 aa, Artificial Sequence
  Description of Artificial Sequence Synthetic peptide
QQHYSTPFT

SEQ ID NO: 59 - protein, 116 aa, Artificial Sequence
  Description of Artificial Sequence Synthetic polypeptide
EVKLEESGGGLVQPGGSMKLSCVASGFTFSNFWMDWVRQSPEKGLEWVAEIRLKSTNYATHYAESVKGRFTISRDDSKSSVYLQMNNLRAEDTGIYYCTATSTGYWGQGTTLTVSS

SEQ ID NO: 60 - protein, 5 aa, Artificial Sequence
  Description of Artificial Sequence Synthetic peptide
NFWMD

SEQ ID NO: 61 - protein, 19 aa, Artificial Sequence
  Description of Artificial Sequence Synthetic peptide
EIRLKSTNYATHYAESVKG

SEQ ID NO: 62 - protein, 5 aa, Artificial Sequence
  Description of Artificial Sequence Synthetic peptide
TSTGY

SEQ ID NO: 63 - protein, 107 aa, Artificial Sequence
  Description of Artificial Sequence Synthetic polypeptide
SIVMTQSPKILLVSAGDRVTITCKASQSVSNDVAWYQQKPGQSPKLLIYYASNRYTGVPDRFTGSGYGTDFTFTISTVQAEDLAVYFCQQDYSSPYTFGGGTKLEIK

SEQ ID NO: 64 - protein, 11 aa, Artificial Sequence
  Description of Artificial Sequence Synthetic peptide
KASQSVSNDVA

SEQ ID NO: 65 - protein, 7 aa, Artificial Sequence
  Description of Artificial Sequence Synthetic peptide
YASNRYT

SEQ ID NO: 66 - protein, 9 aa, Artificial Sequence
  Description of Artificial Sequence Synthetic peptide
QQDYSSPYT

SEQ ID NO: 67 - DNA, 348 bp, Artificial Sequence
  Description of Artificial Sequence Synthetic polynucleotide
gaagtgaaactggaagaaagcggcggcggcctggtgcagccgggcggcagcatgaaactgagctgcgtggcgagcggctttacctttagcaacttttggatggattgggtgcgccagagcccggaaaaaggcctggaatgggtggcggaaattcgcctgaaaagcaccaactatgcgacccattatgcggaaagcgtgaaaggccgctttaccattagccgcgatgatagcaaaagcagcgtgtatctgcagatgaacaacctgcgcgcggaagataccggcatttattattgcaccgcgaccagcaccggctattggggccagggcaccaccctgaccgtgagcagc

SEQ ID NO: 68 - DNA, 12 bp, Artificial Sequence
  Description of Artificial Sequence Synthetic oligonucleotide
aacttttggatg

SEQ ID NO: 69 - DNA, 57 bp, Artificial Sequence
  Description of Artificial Sequence Synthetic oligonucleotide
gaaattcgcctgaaaagcaccaactatgcgacccattatgcggaaagcgtgaaaggc

SEQ ID NO: 70 - DNA, 12 bp, Artificial Sequence
  Description of Artificial Sequence Synthetic oligonucleotide
accagcaccggc

SEQ ID NO: 71 - DNA, 321 bp, Artificial Sequence
  Description of Artificial Sequence Synthetic polynucleotide
agcattgtgatgacccagagcccgaaaattctgctggtgagcgcgggcgatcgcgtgaccattacctgcaaagcgagccagagcgtgagcaacgatgtggcgtggtatcagcagaaaccgggccagagcccgaaactgctgatttattatgcgagcaaccgctataccggcgtgccggatcgctttaccggcagcggctatggcaccgattttacctttaccattagcaccgtgcaggcggaagatctggcggtgtatttttgccagcaggattatagcagcccgtatacctttggcggcggcaccaaactggaaattaaa

SEQ ID NO: 72 - DNA, 33 bp, Artificial Sequence
  Description of Artificial Sequence Synthetic oligonucleotide
aaagcgagccagagcgtgagcaacgatgtggcg

SEQ ID NO: 73 - DNA, 21 bp, Artificial Sequence
  Description of Artificial Sequence Synthetic oligonucleotide
tatgcgagcaaccgctatacc

SEQ ID NO: 74 - DNA, 27 bp, Artificial Sequence
  Description of Artificial Sequence Synthetic oligonucleotide
cagcaggattatagcagcccgtatacc

SEQ ID NO: 75 - protein, 123 aa, Artificial Sequence
  Description of Artificial Sequence Synthetic polypeptide
QVQLQQPGAEIVRPGASVKLSCKASGYTFTDYWMNWVKLRPGQGLEWIGIIDPSDSYASYNQKFKGKATLTVDESSSTAYMQLSSLTSEDSAVYYCARKSYYGGNYYYAMDYWGQGTSVTVSS

SEQ ID NO: 76 - protein, 5 aa, Artificial Sequence
  Description of Artificial Sequence Synthetic peptide
DYWMN

SEQ ID NO: 77 - protein, 17 aa, Artificial Sequence
  Description of Artificial Sequence Synthetic peptide
IIDPSDSYASYNQKFKG

SEQ ID NO: 78 - protein, 14 aa, Artificial Sequence
  Description of Artificial Sequence Synthetic peptide
KSYYGGNYYYAMDY

SEQ ID NO: 79 - protein, 108 aa, Artificial Sequence
  Description of Artificial Sequence Synthetic polypeptide
EIVLTQSPALMAASPGEKVTITCSVSSSINSINLHWYRQKSETSPKPWIYGTSNLASGVPVRFSGSGSGTSYSLTISSMEAEDAATYYCQQWSSYPLTFGAGTKLELK

SEQ ID NO: 80 - protein, 12 aa, Artificial Sequence
  Description of Artificial Sequence Synthetic peptide
SVSSSINSINLH

SEQ ID NO: 81 - protein, 7 aa, Artificial Sequence
  Description of Artificial Sequence Synthetic peptide
GTSNLAS

SEQ ID NO: 82 - protein, 9 aa, Artificial Sequence
  Description of Artificial Sequence Synthetic peptide
QQWSSYPLT

SEQ ID NO: 83 - DNA, 369 bp, Artificial Sequence
  Description of Artificial Sequence Synthetic polynucleotide
caggtccaactgcagcagcctggggctgagattgtgaggcctggggcttcagtgaagctgtcctgcaaggcttctggctacaccttcaccgactattggatgaactgggtgaaactgaggcctggacaaggccttgagtggattggaataattgatccttctgatagttatgctagctacaatcaaaagttcaagggcaaggccacattgactgtagacgagtcctccagcacagcctacatgcagctcagcagcctgacatctgaggactctgcggtctattactgtgcaagaaaatcttactatggtggtaactactactatgctatggactactggggtcaaggaacctcagtcaccgtctcctca

SEQ ID NO: 84 - DNA, 15 bp, Artificial Sequence
  Description of Artificial Sequence Synthetic oligonucleotide
gactattggatgaac

SEQ ID NO: 85 - DNA, 51 bp, Artificial Sequence
  Description of Artificial Sequence Synthetic oligonucleotide
ataattgatccttctgatagttatgctagctacaatcaaaagttcaagggc

SEQ ID NO: 86 - DNA, 42 bp, Artificial Sequence
  Description of Artificial Sequence Synthetic oligonucleotide
aaatcttactatggtggtaactactactatgctatggactac

SEQ ID NO: 87 - DNA, 324 bp, Artificial Sequence
  Description of Artificial Sequence Synthetic polynucleotide
gaaattgtgctcacccagtctccagcactcatggctgcatctccaggggagaaggtcaccatcacctgcagtgtcagctcaagtataaattccatcaacttgcactggtaccggcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaa

SEQ ID NO: 88 - DNA, 36 bp, Artificial Sequence
  Description of Artificial Sequence Synthetic oligonucleotide
agtgtcagctcaagtataaattccatcaacttgcac

SEQ ID NO: 89 - DNA, 21 bp, Artificial Sequence
  Description of Artificial Sequence Synthetic oligonucleotide
ggcacatccaacctggcttct

SEQ ID NO: 90 - DNA, 27 bp, Artificial Sequence
  Description of Artificial Sequence Synthetic oligonucleotide
caacagtggagtagttacccactcacg

SEQ ID NO: 91 - protein, 471 aa, Artificial Sequence
  Description of Artificial Sequence Synthetic polypeptide
MKCSWVMFLVATATGVNSQVQLQQPGAEIVRPGASVKLSCKASGYTFTDYWMNWVKLRPGQGLEWIGIIDPSDSYASYNQKFKGKATLTVDESSSTAYMQLSSLTSEDSAVYYCARKSYYGGNYYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 92 - DNA, 1412 bp, Artificial Sequence
  Description of Artificial Sequence Synthetic polynucleotide
atgaaatgcagctgggtcatgttcttggtagcaacagctacaggtgtcaactcccaggtccaactgcagcagcctggggctgagattgtgaggcctggggcttcagtgaagctgtcctgcaggcttctggctacaccttcaccgactattggatgaactgggtgaaactgaggcctggacaaggccttgagtggattggaataattgatccttctgatagttatgctagctacaatcaaaagttcaagggcaaggccacattgactgtagacgagtcctccagcacagcctacatgcagctcagcagcctgacatctgaggactctgcggtctattactgtgcaagaaaatcttactatggtggtaactactactatgctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 93 - protein, 215 aa, Artificial Sequence
  Description of Artificial Sequence Synthetic polypeptide
EIVLTQSPALMAASPGEKVTITCSVSSSINSINLHWYRQKSETSPKPWIYGTSNLASGVPVRFSGSGSGTSYSLTISSMEAEDAATYYCQQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 94 - DNA, 645 bp, Artificial Sequence
  Description of Artificial Sequence Synthetic polynucleotide
gaaattgtgctcacccagtctccagcactcatggctgcatctccaggggagaaggtcaccatcacctgcagtgtcagctcaagtataaattccatcaacttgcactggtaccggcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 95 - protein, 19 aa, Artificial Sequence
  Description of Artificial Sequence Synthetic consensus sequence
XIRLKSXNYATHYAESVKG

SEQ ID NO: 96 - protein, 5 aa, Artificial Sequence
  Description of Artificial Sequence Synthetic consensus sequence
XXXXY

SEQ ID NO: 97 - protein, 11 aa, Artificial Sequence
  Description of Artificial Sequence Synthetic consensus sequence
KASQXVSXDVA

SEQ ID NO: 98 - protein, 7 aa, Artificial Sequence
  Description of Artificial Sequence Synthetic consensus sequence
XASXRYT

SEQ ID NO: 99 - protein, 9 aa, Artificial Sequence
  Description of Artificial Sequence Synthetic consensus sequence
QQXYSXPXT

SEQ ID NO: 100 - protein, 11 aa, Homo sapiens
WGQGTTLTVSS

SEQ ID NO: 101 - protein, 11 aa, Homo sapiens
WGRGTLVTVSS

SEQ ID NO: 102 - protein, 100 aa, Artificial Sequence
  Description of Artificial Sequence Synthetic polypeptide
EVKLEESGGGLVQPGGSMKLSCVASGFTFSNFWMDWVRQSPEKGLEWVAEIRLKSTNYATHYAESVKGRFTISRDDSKSSVYLQMNNLRAEDTGIYYCTA

SEQ ID NO: 103 - protein, 100 aa, Artificial Sequence
  Description of Artificial Sequence Synthetic polypeptide
EVKLEESGGGLVQPGGSMKLSCVASGFTFSXXXMDWVRQSPEKGLEWVAEIXXXXXXXXXHYAXXVXXRFTISRDDSKSSVYLQMNNLRAEDTGIYYCTA

SEQ ID NO: 104 - protein, 100 aa, Artificial Sequence
  Description of Artificial Sequence Synthetic polypeptide
EVKLEESGGGLVQPGGSMKLSCVASGFTFSXXXXXWVRQSPEKGLEWVAXXXXXXXXXXXXXXXXXXXRFTISRDDSKSSVYLQMNNLRAEDTGIYYCTA

SEQ ID NO: 105 - protein, 100 aa, Artificial Sequence
  Description of Artificial Sequence Synthetic polypeptide
XVXLXXXXXXXXXXXXXXXXXXXAXGFXFXXXXMDXVXQXXXXGLXWVAEIXXXXXXXXXHYAXXVXXXFXIXRXDXXSXVXXXXXXXXXXXXXXXYXTA

SEQ ID NO: 106 - protein, 100 aa, Artificial Sequence
  Description of Artificial Sequence Synthetic polypeptide
XVXLXXXXXXXXXXXXXXXXXXXAXGFXFXXXXXXXVXQXXXXGLXWVAXXXXXXXXXXXXXXXXXXXXFXIXRXDXXSXVXXXXXXXXXXXXXXXYXTA

SEQ ID NO: 107 - protein, 100 aa, Artificial Sequence
  Description of Artificial Sequence Synthetic polypeptide
XVXLXXXXXXXXXXXXXXXXXXXAXGFXFXNFWMDXVXQXXXXGLXWVAEIRLKSTNYATHYAESVKGXFXIXRXDXXSXVXXXXXXXXXXXXXXXYXTA

SEQ ID NO: 108 - protein, 101 aa, Artificial Sequence
  Description of Artificial Sequence Synthetic polypeptide
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTRX

SEQ ID NO: 109 - protein, 20 aa, Artificial Sequence
  Description of Artificial Sequence Synthetic peptide
YYYYYGMDVWGQGTTVTVSS

SEQ ID NO: 110 - protein, 23 aa, Artificial Sequence
  Description of Artificial Sequence Synthetic peptide
GGLVQPGGSLKLSCAASGFTFSN

SEQ ID NO: 111 - protein, 23 aa, Artificial Sequence
  Description of Artificial Sequence Synthetic peptide
GGLVQPGGSLKLSCAASGFTFSG

SEQ ID NO: 112 - protein, 15 aa, Artificial Sequence
  Description of Artificial Sequence Synthetic peptide
ASGFTFSGSAMHWVR

SEQ ID NO: 113 - protein, 20 aa, Artificial Sequence
  Description of Artificial Sequence Synthetic peptide
FSNFWMDWVRQASGKGLEWV

SEQ ID NO: 114 - protein, 16 aa, Artificial Sequence
  Description of Artificial Sequence Synthetic peptide
AMHWVRQASGKGLEWV

SEQ ID NO: 115 - protein, 19 aa, Artificial Sequence
  Description of Artificial Sequence Synthetic peptide
GLEWVGEIRLKSTNYATHY

SEQ ID NO: 116 - protein, 19 aa, Artificial Sequence
  Description of Artificial Sequence Synthetic peptide
GLEWVAEIRLKSTNYATHY

SEQ ID NO: 117 - protein, 19 aa, Artificial Sequence
  Description of Artificial Sequence Synthetic peptide
GLEWVGRIRSKANSYATAY

SEQ ID NO: 118 - protein, 10 aa, Homo sapiens
FGGGTKLEIK

SEQ ID NO: 119 - protein, 10 aa, Homo sapiens
FGQGTKLEIK

SEQ ID NO: 120 - protein, 10 aa, Homo sapiens
FGPGTKVDIK

SEQ ID NO: 121 - protein, 10 aa, Homo sapiens
FGQGTRLEIK

SEQ ID NO: 122 - protein, 95 aa, Artificial Sequence
  Description of Artificial Sequence Synthetic polypeptide
SIVMTQSPKILLVSAGDRVTITCKASQSVSNDVAWYQQKPGQSPKLLIYYASNRYTGVPDRFTGSGYGTDFTFTISTVQAEDLAVYFCQQDYSSP

SEQ ID NO: 123 - protein, 95 aa, Artificial Sequence
  Description of Artificial Sequence Synthetic polypeptide
SIVMTQSPKILLVSAGDRVTITCXAXXXVXXXVAWYQQKPGQSPKLLIYXXXXXYXGVPDRFTGSGYGTDFTFTISTVQAEDLAVYFCQQDXXSP

SEQ ID NO: 124 - protein, 95 aa, Artificial Sequence
  Description of Artificial Sequence Synthetic polypeptide
SIVMTQSPKILLVSAGDRVTITCXXXXXXXXXXXWYQQKPGQSPKLLIYXXXXXXXGVPDRFTGSGYGTDFTFTISTVQAEDLAVYFCXXXXXXX

SEQ ID NO: 125 - protein, 95 aa, Artificial Sequence
  Description of Artificial Sequence Synthetic polypeptide
XIXMXXXXXXXXXXXXXXXXXXXXAXXXVXXXVAXYXQXXXXSPXLLIYXXXXXYXXVXXXFXGXXXXXXFXXXXXXXXXXXXXXXFXQQDXXSP

SEQ ID NO: 126 - protein, 95 aa, Artificial Sequence
  Description of Artificial Sequence Synthetic polypeptide
XIXMXXXXXXXXXXXXXXXXXXXXXXXXXXXXXXXYXQXXXXSPXLLIYXXXXXXXXVXXXFXGXXXXXXFXXXXXXXXXXXXXXXFXXXXXXXX

SEQ ID NO: 127 - protein, 95 aa, Artificial Sequence
  Description of Artificial Sequence Synthetic polypeptide
XIXMXXXXXXXXXXXXXXXXXXXKASQSVSNDVAXYXQXXXXSPXLLIYYASNRYTXVXXXFXGXXXXXXFXXXXXXXXXXXXXXXFXQQDYSSP

SEQ ID NO: 128 - protein, 95 aa, Artificial Sequence
  Description of Artificial Sequence Synthetic consensus sequence
DIQMTQSPSSLSASVGDRVTITCRASQGISSYLAWYQQKPGKAPKLLIYAASSLQSGVPSRFSGSGSGTDFTLTISSLQPEDFATYYCQQYNSYP

SEQ ID NO: 129 - protein, 95 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 130 - protein, 95 aa, Artificial Sequence; identical to SEQ ID NO: 129
  Description of Artificial Sequence Synthetic polypeptide

SEQ ID NO: 131 - protein, 95 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 132 - protein, 95 aa, Artificial Sequence
  Description of Artificial Sequence Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 133 - protein, 95 aa, Artificial Sequence
  Description of Artificial Sequence Synthetic polypeptide
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 134 - protein, 95 aa, Artificial Sequence
  Description of Artificial Sequence Synthetic polypeptide
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 135 - protein, 95 aa, Artificial Sequence
  Description of Artificial Sequence Synthetic polypeptide
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 136 - protein, 96 aa, Artificial Sequence
  Description of Artificial Sequence Synthetic polypeptide
DIQLTQSPSSLSASVGDRVTITCRVSQGISSYLNWYRQKPGKVPKLLIYSASNLQSGVPSRFSGSGSGTDFTLTISSLQPEDVATYYGQRTYNAPP

SEQ ID NO: 137 - protein, 95 aa, Artificial Sequence
  Description of Artificial Sequence Synthetic polypeptide
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 138 - protein, 95 aa, Artificial Sequence
  Description of Artificial Sequence Synthetic polypeptide
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 139 - protein, 95 aa, Artificial Sequence
  Description of Artificial Sequence Synthetic polypeptide
AIRMTQSPSSFSASTGDRVTITCRASQGISSYLAWYQQKPGKAPKLLIYAASTLQSGVPSRFSGSGSGTDFTLTISCLQSEDFATYYCQQYYSYP

SEQ ID NO: 140 - protein, 95 aa, Artificial Sequence
  Description of Artificial Sequence Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 141 - protein, 95 aa, Artificial Sequence
  Description of Artificial Sequence Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNNYP

SEQ ID NO: 142 - protein, 95 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 143 - protein, 95 aa, Artificial Sequence
  Description of Artificial Sequence Synthetic polypeptide
NIQMTQSPSAMSASVGDRVTITCRARQGISNYLAWFQQKPGKVPKHLIYAASSLQSGVPSRFSGSGSGTEFTLTISSLQPEDFATYYCLQHNSYP

SEQ ID NO: 144 - protein, 96 aa, Artificial Sequence
  Description of Artificial Sequence Synthetic polypeptide
DIQMIQSPSFLSASVGDRVSIICWASEGISSNLAWYLQKPGKSPKLFLYDAKDLHPGVSSRFSGRGSGTDFTLTIISLKPEDFAAYYCKQDFSYPP

SEQ ID NO: 145 - protein, 95 aa, Artificial Sequence
  Description of Artificial Sequence Synthetic polypeptide
AIRMTQSPFSLSASVGDRVTITCWASQGISSYLAWYQQKPAKAPKLFIYYASSLQSGVPSRFSGSGSGTDYTLTISSLQPEDFATYYCQQYYSTP

SEQ ID NO: 146 - protein, 95 aa, Artificial Sequence
  Description of Artificial Sequence Synthetic polypeptide
VIWMTQSPSLLSASTGDRVTISCRMSQGISSYLAWYQQKPGKAPELLIYAASTLQSGVPSRFSGSGSGTDFTLTISCLQSEDFATYYCQQYYSFP

SEQ ID NO: 147 - protein, 96 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPP

SEQ ID NO: 148 - protein, 96 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGRDFTLTISSLEPEDFAVYYCQQRSNWPP

SEQ ID NO: 149 - protein, 90 aa, Artificial Sequence
  Description of Artificial Sequence Synthetic polypeptide
EIVLTQSPATLSLSPGERATLSCRASQSVSSYLAWYQQKPGQAPRLLIYGASTRATGIPARFSGSGSGTEFTLTISSLQSEDFAVYYCQX

SEQ ID NO: 150 - protein, 90 aa, Artificial Sequence
  Description of Artificial Sequence Synthetic polypeptide
EIVLTQSPATLSLSPGERATLSCRASQGVSSYLAWYQQKPGQAPRLLIYDASSRATGIPARFSGSGPGTDFTLTISSLEPEDFAVYYCQX

SEQ ID NO: 151 - protein, 95 aa, Artificial Sequence
  Description of Artificial Sequence Synthetic polypeptide
EIVLTQSPATLSLSPGERATLSCRASQGVSSNLAWYQQKPGQAPRLLIYDASNRATGIPARFSGSGPGTDFTLTISSLEPEDFAVYYCQQRSNWH

SEQ ID NO: 152 - protein, 96 aa, Artificial Sequence
  Description of Artificial Sequence Synthetic polypeptide
EIVLTQSPATLSLSPGERATLSCRASQGVSSYLAWYQQKPGQAPRLLIYDASNRATGIPARFSGSGPGTDFTLTISSLEPEDFAVYYCQQRSNWHP

SEQ ID NO: 153 - protein, 96 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPP

SEQ ID NO: 154 - protein, 96 aa, Artificial Sequence; identical to SEQ ID NO: 153
  Description of Artificial Sequence Synthetic polypeptide

SEQ ID NO: 155 - protein, 94 aa, Artificial Sequence
  Description of Artificial Sequence Synthetic consensus sequence
EIVLTQSPATLSLSPGERATLSCRASQSVSSYLAWYQQKPGQAPRLLIYDASNRATGIPARFSGSGSGTDFTLTISSLEPEDFAVYYCQQNWPP

SEQ ID NO: 156 - protein, 12 aa, Artificial Sequence
  Description of Artificial Sequence Synthetic peptide
LTFGGGTKVEIK

SEQ ID NO: 157 - protein, 15 aa, Artificial Sequence
  Description of Artificial Sequence Synthetic peptide
DIQMTQSPSSLSASV

SEQ ID NO: 158 - protein, 16 aa, Artificial Sequence
  Description of Artificial Sequence Synthetic peptide
APKLLIYDASNLETGV

SEQ ID NO: 159 - protein, 24 aa, Artificial Sequence
  Description of Artificial Sequence Synthetic peptide
APKLLIYYASNRYTGVPSRFSGSG

SEQ ID NO: 160 - protein, 24 aa, Artificial Sequence
  Description of Artificial Sequence Synthetic peptide
SPKLLIYYASNRYTGVPSRFSGSG

SEQ ID NO: 161 - protein, 19 aa, Artificial Sequence
  Description of Artificial Sequence Synthetic peptide
GTDFTFTISSLQPEDIATY

SEQ ID NO: 162 - protein, 15 aa, Artificial Sequence
  Description of Artificial Sequence Synthetic peptide
EIVMTQSPATLSVSP

SEQ ID NO: 163 - protein, 24 aa, Artificial Sequence
  Description of Artificial Sequence Synthetic peptide
APRLLIYYASNRYTGIPARFSGSG

SEQ ID NO: 164 - protein, 24 aa, Artificial Sequence
  Description of Artificial Sequence Synthetic peptide
SPRLLIYYASNRYTGVPARFSGSG

SEQ ID NO: 165 - protein, 15 aa, Artificial Sequence
  Description of Artificial Sequence Synthetic peptide
GTEFTLTISSLQSED

SEQ ID NO: 166 - protein, 19 aa, Artificial Sequence
  Description of Artificial Sequence Synthetic peptide
KSTVYLQMNSLKTEDTAVY

SEQ ID NO: 167 - protein, 18 aa, Artificial Sequence
  Description of Artificial Sequence Synthetic peptide
NTAYLQMNSLKTEDTAVY